(19) United States
(12) Patent Application Publication
(10) Pub. No.: US 2021/0040470 A1
(43) Pub. Date: Feb. 11, 2021

(54) AN ENZYME CONTAINING POLYMER, A SENSOR CONTAINING THE SAME, A MONITOR AND A MONITORING METHOD

(71) Applicant: SINGAPORE HEALTH SERVICES PTE LTD, Singapore (SG)

(72) Inventors: Ngian Chye Tan, Singapore (SG); Christopher Hoe Kong Chui, Singapore (SG); Zanzan Zhu, Singapore (SG); Wai Chye Cheong, Singapore (SG); Fiona Wei Ling Loke, Singapore (SG); John Shen Him Ng, Singapore (SG); Lu Gan, Singapore (SG); Richard Siang-Long Lieu, Singapore (SG)

(21) Appl. No.: 16/979,695

(22) PCT Filed: Apr. 10, 2019
(86) PCT No.: PCT/SG2019/050202

§ 371 Date: Sep. 10, 2020

(30) Foreign Application Priority Data
Apr. 10, 2018 (SG) 10201802969S

(51) Int. Cl. C12N 11/10; C12N 11/06; C08L 5/08; C08L 79/02; G01N 27/327

(52) U.S. Cl. CPC C12N 11/10; C12N 11/06; G01N 27/3275; C08L 79/02; C08L 5/08

(57) ABSTRACT

The present invention relates to an enzyme-containing polymer of 2-amino monosaccharide, preferably an enzyme-containing chitosan, comprising: a first repeating unit of the following Formula 1a: a second repeating unit of the following Formula 1b: and a third repeating unit of the following Formula 1c: or conjugate salts thereof, wherein all the substituents are as defined herein. There is also provided a redox polymer and the methods of preparing the enzyme-containing polymer and the redox polymer. The present invention also relates to a sensor, a method of manufacturing the sensor, a monitor, methods for monitoring failure of a tissue and uses of the sensor comprising the enzyme-containing polymer, and the monitor thereof.

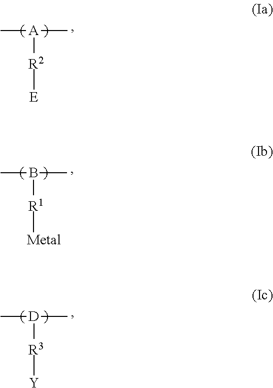

### CROSS-REFERENCE TO RELATED APPLICATION

This application claims priority to Singapore application number 10201802969S filed on 10 Apr. 2018, the disclosure of which is hereby incorporated by reference.

### TECHNICAL FIELD

The present disclosure generally relates to an enzyme containing polymer, a method for synthesizing the enzyme containing polymer, a sensor comprising the enzyme containing polymer, a device comprising the enzyme containing polymer, a monitor and a method for post-surgical monitoring of a vascularized graft using an electrochemical metabolite detector, which can be conducted in an automated continuous manner.

### BACKGROUND ART

After a traumatic accident or cancerous tissue removal, surgeons commonly perform reconstruction of a patient's body using vascularized skin and muscle grafts, also called flaps. These flaps are usually transferred with one or more veins and one artery from part of the patient's own body to the site of the previously removed tissue. The blood vessels are then reconnected under the microscope. In the head and neck region, reconstruction of pharyngeal and oesophageal defects may be done using flaps such as the anterolateral thigh flap (ALT). These flaps are often ‘buried’ deep within a cavity and cannot be easily monitored clinically.

One of the postoperative complications of major concern to surgeons is flap death/failure, commonly a result of vascular thrombosis. Venous thrombosis accounts for the majority of flap failures, while arterial thrombosis is less common. The consequences of a failed flap are considered by surgeons to be devastating due to the partial or complete loss of the flap, necessitating an extended hospital stay for a repeat surgery to replace the failed flap, as well as complications of the failed flap itself. Flap surgery, which is usually combined with other procedures such as excision of cancerous tissue, is a long procedure often taking between 8 to 12 hours. In addition, such flap surgeries are expensive (typically ranging in the tens of thousands) and such costs can double if the flap fails. Due to the high investment of time and cost in the initial flap surgery and the morbidity associated with flap failure, surgeons generally keep patients in hospital for at least 7 days after a free flap surgery in order to monitor the flaps. Once vascular compromise is suspected, immediate reexploration and salvage is done. However, the longer the time taken to detect flap failure, the lower the salvage rate will be.

Nurses and doctors take on the labour-intensive task of hourly monitoring of the flaps for a minimum of 24 to 48 hours. Flap perfusion is manually monitored by observation of clinical signs such as colour, temperature, turgidity, bleeding response to a needle stick and Doppler signal. Observation of clinical signs can only be done with surface flaps, where only part of the flap is visible. The problem is exacerbated when the flap is buried, and none of it is visible. Head and neck reconstructions frequently involve buried flaps, which are used to reconstruct part of the oesophagus or pharynx. There is currently no widely accepted way to monitor buried flaps.

A 2007 study showed that current monitoring methods are flap colour change (utilized by 79.4% of surgeons), Doppler signal (79.4%), pin prick and bleeding rate (67.6%), capillary refill (61.8%), skin surface temperature (11.8%), and implanted Doppler (8.8%). However, aside from the Doppler signal and implanted Doppler, which present their own challenges and problems. Each still has serious shortcomings for instance, limitations to their uses thus preventing widespread adoption.

Following flap surgeries, the current gold standard of care involves nursing staff or junior clinicians manually monitoring the flap for clinical signs of flap failure, such as a change in colour, temperature, turgidity, capillary refill and/or flow of blood from a prick test. The flap is monitored intensively for the first 48 hours (hourly or more frequently), and then every 4 hours for 5 to 7 days. This is because a majority of failures occur in the initial 2 to 3 days following surgery. The nursing staff or clinician may have to go routinely to the patient's site and evaluate the condition of the flap.

If it is the artery which is blocked, the flap appears pale with a lack of capillary refill. Little blood comes out when the flap is pricked. If it is the vein which is blocked, the flap gets congested with blood and looks blue, with an overly fast capillary refill observed. Upon pricking the flap, dark-coloured blood flows swiftly out.

Clinical observation is time consuming, often labour dependent and risks a potentially delayed response to flap failure. Certain parameters, such as colour changes, are open to interpretation and may be masked in people of darker skin colour. Changes in temperature are slow to occur following a vascular thrombosis and the flap may also be kept deceptively warm by underlying tissue even when it is thrombosed. A manual monitoring method reliant on a visual assessment can lead to ‘human errors’ and inconsistent analysis. The process is rather subjective. The issue of inconsistency in a visual assessment may be compounded if different healthcare staff with varying levels of perception abilities and levels of strain and fatigue conduct manual monitoring by visual assessment over an extended period of time.

The problems with existing gold-standard manual monitoring methods include a reliance on clinical signs, which are subject to interpretation and are only observable hours after vascular thrombosis, making it difficult to salvage the flap within the 8-hour window when the flap may still survive. Another issue is the requirement for trained clinical manpower to perform such manual monitoring on an hourly basis for up to 48 hours, and continuing every few hours for up to 5 days postoperatively, despite an already high and increasing patient care load. The process of manual monitoring adds healthcare costs in terms of ‘staff man hours’, is laborious and workflow-dependent and typically presents a strain on already busy hospital systems.

Between 5 and 25% of flaps may require re-exploration due to circulatory compromise, and the flap suspected to be compromised is surgically accessed for immediate intervention if necessary. Nonetheless, flap salvage success rates vary. In one study of 1310 flaps with 49 compromised cases, the salvage rate was 44.9%. Another study found the salvage rate to be between 33 to 57% of cases. There is clearly room for improvement.

In the United States, a 5-year study noted that among 95 surgeons surveyed, 90% relied on adjunctive monitoring devices in addition to clinical assessment. The two most common monitoring approaches were laser Doppler (28%) and regular Doppler (29%) respectively. These approaches provide flow information that complements the hourly manual observation but cannot replace or do away with the manual observation process as it stands.

For some time, implantable Doppler probes have been the only feasible method to use for monitoring buried flaps. These implantable probes are sutured around the blood vessel at the anastomosis site, where the vessels supplying the flap are joined, and later removed by forcibly pulling them out.

However, a study of 20 buried flaps found that the implantable Doppler probes suffered from a false-positive rate as high as 88%, although the sensitivity was 100%. Another study using the implantable Doppler probes with 96 buried and non-buried flaps found that there was a 31% false-positive rate, leading to unnecessary re-exploration. Unnecessary re-exploration is taxing not only for the patient but also the physicians. The situation is undesirable and ineffective.

Any alarm from the implantable Doppler probe immediately triggers a response from the surgical team which will lead to a surgical re-intervention. A high false-positive rate would mean that a large number of unnecessary surgical re-explorations are performed resulting in unneeded additional cost, trauma and hospitalization time for the patients. As a result of the poor specificity associated with the implantable Doppler, surgeons have doubts on its reliability. The implantable Doppler probes are thus not a highly useful tool in clinical practice. Further, the need to pull out the probe after it has been placed at such a sensitive area as the anastomosis site, where the vessels are joined, carries the risk that the anastomosis may be damaged or vessel thrombosis may result due to the force of removal. This can aggravate what is likely to be an already complex clinical situation. Due to grave concerns over the safety of the implantable Doppler probes, there is great reluctance amongst surgeons to use it.

As for the regular handheld Doppler probe, surgeons noted that there was difficulty in targeting the right vessel to monitor. The presence of other major vessels nearby might lead to false readings. In the study previously mentioned, one flap being monitored with a handheld Doppler probe had a Doppler signal despite being already necrotic. Such events, when added to the inconvenience of requiring a trained person to operate the Doppler probe at the bedside during each monitoring session, reduce the confidence level of clinicians in the Doppler probes and often give rise to the view that the Doppler probe is at best an adjunct to manual monitoring for surface flaps. Doppler probes are thus seen as a method of last resort for assessment and monitoring of buried flaps, where there is no real useful alternative available.

The success of microvascular flap salvage is inversely proportional to the time between the beginning of flap ischemia and its recognition by clinicians. There is also a limited window of time within which it is possible to salvage the flap after vessel occlusion. For jejunal flaps, which are more susceptible to ischemia, full-thickness necrosis takes place within 6 hours of venous thrombosis or compromise. For others, flap survival becomes virtually impossible at 8-12 hours after vessel occlusion first occurs due to lack of perfusion. After this time, reestablishment of blood flow no longer results in perfusion; a phenomenon termed the “no-reflow” phenomenon. Hence, monitoring of the flap is of paramount importance to enable early intervention. Unfortunately, clinical signs of flap failure generally become clear to human observation only hours after vascular thrombosis have taken place.

There is therefore an unmet need for an accurate, safe, and labour-saving method of monitoring both buried and surface flaps. The high morbidity and mortality associated with flap loss, as well as the limited capacity of medical staff to undertake manual flap monitoring, lead to a compelling clinical need for such a device.

Monitoring of glucose level is one of the most inexpensive methods which would change significantly within minutes of vessel thrombosis. It has been shown that high sensitivity and specificity in regards to flap monitoring in animal trials can be obtained, whereby this process can be automated. Indeed in a study of adult rats with complete arterial and venous occlusion, interstitial glucose declined significantly, and sensitivity and specificity of flap failure detection by glucose testing were at 100% and 95%, respectively. Moreover, to cater to flaps of varying composition, studies have shown that measurement of glucose alone is insufficient (flaps consisting of mostly muscle tissue showed a more pronounced drop in glucose levels during ischemia than those composed mostly of fat and skin). That is why studies on lactate as an additional tool for accurate flap monitoring have been proposed.

Electrochemical sensors were chosen for glucose sensing due to their intrinsic advantages such as high sensitivity, fast response, easy operation, and favorable portability. The first generation of glucose biosensors was based on an electrochemical reaction that relies on the enzyme glucose oxidase (GOx). GOx catalyses the oxidation of glucose to gluconolactone by molecular oxygen while producing hydrogen peroxide (H₂O₂) and water as by-products. Gluconolactone further undergoes a reaction with water to produce the carboxylic acid product, gluconic acid. In order to carry out the oxidation reactions, GOx requires a redox cofactor—flavin adenine dinucleotide (FAD⁺). FAD⁺ works as the initial electron acceptor which becomes reduced to FADH₂ during the redox reaction. The FAD⁺ cofactor is regenerated by the subsequent reaction with oxygen to produce H₂O₂. This reaction occurs at the anode, where the number of transferred electrons can be easily recognized and this electron flow is correlated to the quantity of H₂O₂ produced and hence the concentration of glucose. GOx is the standard enzyme for glucose biosensors, where it is affordable and has high selectivity for glucose. Other enzymes can be used for glucose sensing including hexokinase and glucose-1-dehydrogenase (GDH).

The concept of lactate sensing is the same as glucose sensing, where the most commonly used enzyme in lactate sensors are L-lactate dehydrogenase (LDH) and L-lactate oxidase (LOx), due to the relatively simple enzymatic reaction and simple sensor design fabrication. LOx can catalyse the oxidation of L-lactate to pyruvate in the presence of dissolved oxygen and forms hydrogen peroxide (H₂O₂).

However, there are some drawbacks for the first-generation of glucose/lactate biosensors: 1) the amperometric measurement of hydrogen peroxide required a high operation potential for high selectivity; and 2) the restricted solubility of oxygen in biological fluids. The improvements can be achieved by replacing oxygen with redox mediators that are able to carry electrons from the enzyme to the surface of the working electrode. Meanwhile, the active centers of enzymes are surrounded by a thick protein layer and are located deeply in hydrophobic cavity of molecules, where the direct electrochemistry of enzyme is very difficult. Therefore, the use of an electrical connector can enhance the transportation of electrons. Redox mediators are natural or artificial compounds used as electron acceptors and electron donors during electron transfer. There are several examples of redox mediators such as ferrocene derivatives, ferricyanide, quinines, osmium complexes, ruthenium complex and many others. For implantable glucose/lactate sensing applications, the most commonly used polymer is biocompatible polymer containing redox mediators.

In this regard, there is also a need to provide a sensor having a successful electron redox properties or redox-active mediator that overcomes or at least ameliorates, one or more of the disadvantages described above.

### SUMMARY

According to an aspect, there is provided an enzyme-containing polymer comprising:

a first repeating unit of Formula Ia:

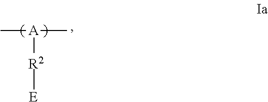

a second repeating unit of Formula Ib:

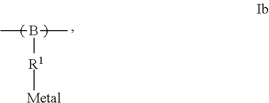

and

a third repeating unit of Formula Ic:

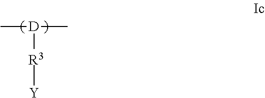

or conjugate salts thereof, wherein

each of A, B, and D is independently a 2-amino monosaccharide;

E is an enzyme comprising an n-terminal amine and optionally one or more lysine residues, wherein R² is covalently bonded to the n-terminal amine or the amine side chain of the one or more lysine residues;

Metal is a metal complex having a redox potential lower than hydrogen peroxide under physiological conditions;

R¹ is —N*(R)(C═O)—, —N*(R)(C═O)N(R)—, —N*(R)(CR₂)ₙ—, —N*(R)(CR₂)ₙ(C═O)—, —N*(R)(C═O)(CR₂)ₙ—, —N*(R)(C═O)(CR₂)ₙ(C═O)—, —N*(R)(CR₂)ₙO(CR₂)ₘ—, —N*(R)(CR₂)ₙS(CR₂)ₘ—, —N*(R)(CR₂)ₙO—, —N*(R)(CR₂)ₙ(C═O)O—, —N*(R)(C═O)(CR₂)ₙO—, —N*(R)(C═O)(CR₂)ₙ(C═O)O—, —N*(R)(CR₂)ₙO(CR₂)ₘO—, —N*(R)(CR₂)ₙS(CR₂)ₘO—, —N*═CH(CR₂)ₙ—, —N*═CH(CR₂)ₙ(C═O)—, —N*═CH(CR₂)ₙO(CR₂)ₘ—, —N*═CH(CR₂)ₙS(CR₂)ₘ—, —N*═CH(CR₂)ₙO—, —N*═CH(CR₂)ₙ(C═O)O—, —N*═CH(CR₂)ₙO(CR₂)ₘO—, or —N*═CH(CR₂)ₙS(CR₂)ₘO—, wherein N* represents the nitrogen at the 2 position of the 2-amino monosaccharide;

R² is —N*(R)(CR₂)ₙN**(R)—, —N*(R)(CR₂)ₙ(C═O)N**(R)—, —N*(R)(C═O)(CR₂)ₙ—, —N*(R)(C═O)(CR₂)ₙ(C═O)N**(R)—, —N*(R)(CR₂)ₙO(CR₂)ₘN**(R)—, —N*(R)(CR₂)ₙS(CR₂)ₘN**(R)—, —N*═CH(CR₂)ₙN**(H)—, —N*═CH(CR₂)ₙ(C═O)N**(R)—, —N*═CH(CR₂)ₙO(CR₂)ₘN**(R)—, —N*═CH(CR₂)ₙS(CR₂)ₘN**(R)—, N*(R)(CR₂)ₙCH═N**—, —N*(R)(C═O)(CR₂)ₙCH═N**—, —N*(R)(CR₂)ₙO(CR₂)ₘCH═N**—, —N*(R)(CR₂)ₙS(CR₂)ₘCH═N**—, —N*═CH(CR₂)ₙCH═N**—, —N*═CH(CR₂)ₙO(CR₂)ₘCH═N**—, or —N*═CH(CR₂)ₙS(CR₂)ₘCH═N**—, wherein N* represents the nitrogen at the 2 position of the 2-amino monosaccharide and N** represents the nitrogen of the n-terminal amine or the amine side chain of the one or more lysine residues of the enzyme; or R² is represented by the moiety:

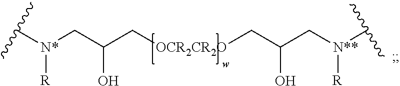

R³ is —N*(R)(CR₂)ₙY, —N*(R)(CR₂)ₙ(C═O)Y, —N*(R)(C═O)(CR₂)ₙY, —N*(R)(C═O)(CR₂)ₙ(C═O)Y, —N*(R)(CR₂)ₙO(CH₂)ₘY, —N*(R)(CR₂)ₙS(CR₂)ₘY, —N*═CH(CR₂)ₙY, —N*═CH(CR₂)ₙ(C═O)Y, —N*═CH(CR₂)ₙO(CR₂)ₘY, —N*═CH(CR₂)ₙS(CR₂)ₘY, —N*(R)(CR₂)ₙCH═Y, —N*(R)(C═O)(CR₂)ₙCH═Y, —N*(R)(CR₂)ₙO(CH₂)ₘCH═Y, —N*(R)(CR₂)ₙS(CR₂)ₘCH═Y, —N*═CH(CR₂)ₙCH═Y, —N*═CH(CR₂)ₙO(CR₂)ₘCH═Y, or —N*═CH(CR₂)ₙS(CR₂)ₘCH═Y, wherein N* represents the nitrogen at the 2 position of the 2-amino monosaccharide; or R³ is represented by the moiety:

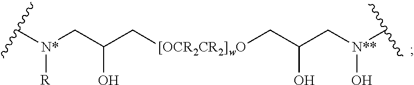

R for each occurrence is independently hydrogen, lower alkyl or hydroxyl;

m for each occurrence is independently a whole number selected between 1-20;

n for each occurrence is independently a whole number selected between 1-20;

w for each occurrence is independently a whole number selected between 1-20; and

Y is a polyalkylamine comprising at least one metal complex, wherein the polyalkylamine optionally crosslinks at least two of the third repeating units.

According to another aspect, there is provided a redox polymer comprising a first repeating unit of Formula IIa:

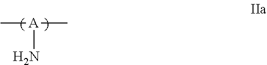

and a second repeating unit of Formula IIb:

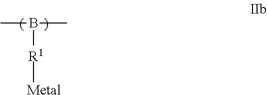

or conjugate salts thereof, wherein

each of A and B independently a 2-amino monosaccharide;

Metal is a metal complex having a redox potential lower than hydrogen peroxide under physiological conditions;

R¹ is —N*(R)(C═O)—, —N*(R)(C═O)N(R)—, —N*(R)(CR₂)ₙ—, —N*(R)(CR₂)ₙ(C═O)—, —N*(R)(C═O)(CR₂)ₙ—, —N*(R)(C═O)(CR₂)ₙ(C═O)—, —N*(R)(CR₂)ₙO(CR₂)ₘ—, —N*(R)(CR₂)ₙS(CR₂)ₘ—, —N*(R)(CR₂)ₙO—, —N*(R)(CR₂)ₙ(C═O)O—, —N*(R)(C═O)(CR₂)ₙO—, —N*(R)(C═O)(CR₂)ₙ(C═O)O—, —N*(R)(CR₂)ₙO(CR₂)ₘO—, —N*(R)(CR₂)ₙS(CR₂)ₘO—, —N*═CH(CR₂)ₙ—, —N*═CH(CR₂)ₙ(C═O)—, —N*═CH(CR₂)ₙO(CH₂)ₘ—, —N*═CH(CR₂)ₙS(CR₂)ₘ—, —N*═CH(CR₂)ₙO—, —N*═CH(CR₂)ₙ(C═O)O—, —N*═CH(CR₂)ₙO(CR₂)ₘO—, or —N*═CH(CR₂)ₙS(CR₂)ₘO—, wherein N* represents the nitrogen at the 2 position of the 2-amino monosaccharide;

R for each occurrence is independently hydrogen or lower alkyl;

m for each occurrence is independently a whole number selected between 1-20; and

n for each occurrence is independently a whole number selected between 1-20.

Advantageously, the enzyme is immobilized into a thin polymer film (of the enzyme containing polymer) with redox properties on the surface of the working electrode through chemical or non-chemical means.

According to another aspect, there is provided a sensor, comprising: a substrate; a first sensor electrode on the substrate; a first sensing layer on the first sensor electrode, the first sensing layer comprising a first enzyme-containing polymer as defined herein; and a reference electrode on the substrate.

According to another aspect, there is provided a monitor, comprising: a receiver module configured to receive an output of a sensor as defined herein; a processor module configured to receive a first metabolite concentration value and a first control value from the receiver module, wherein the processor module is configured to: compare the first metabolite concentration value against the first control value; and generate a first signal based on the comparison.

According to another aspect, there is provided a monitoring system, comprising: a sensor as defined herein; and a monitor as defined here, wherein the receiver of the monitor is arranged in use to receive an output of the sensor.

According to another aspect, there is provided a method of manufacturing a sensor, comprising: providing a substrate; forming a first sensor electrode on the substrate; forming a first sensing layer on the first sensor electrode, the first sensing layer comprising a first enzyme-containing polymer as defined herein; and forming a reference electrode on the substrate.

According to another aspect, there is provided a method for monitoring failure of a tissue on a patient may comprise of the steps:
(i) providing a first sensor as defined herein on or within said tissue, said first sensor being capable of detecting and measuring the amount of a first metabolite;
(ii) providing a second sensor as defined herein on a control region of said patient, said control region being separate from said tissue and wherein said second sensor is capable of detecting and measuring the amount of said first metabolite;
(iii) providing a third sensor as defined herein on or within said tissue, said third sensor being capable of detecting and measuring the amount of a second metabolite and wherein said third sensor is the same as or different from the first sensor;
(iv) providing a fourth sensor as defined herein on said control region of said patient, said fourth sensor being capable of detecting and measuring the amount of said second metabolite and wherein said fourth sensor is the same as or different from the second sensor;
(v) monitoring the amounts of said first metabolite measured by both said first and second sensors for a period of time;
(vi) monitoring the amounts of said second metabolite measured by both said third and fourth sensors for a period of time;

wherein an at least 10% decrease in the amount of said first metabolite as measured by said first sensor as compared to the amount of said first metabolite as measured by said second sensor; and at least 10% increase in the amount of said second metabolite as measured by said third sensor as compared to the amount of said second metabolite as measured by said fourth sensor, is indicative that said tissue is prone to failure.

According to another aspect, there is provided a monitor, comprising: a receiver module configured to receive a sensor output of a sensor and a control output of another sensor; a processor module configured to receive a first metabolite concentration value corresponding to the sensor output and a first control value corresponding to the control output from the receiver module, wherein the processor module is configured to: compare the first metabolite concentration value against the first control value; and generate a first alarm signal on a condition that a difference between the first metabolite concentration value and the first control value is above a first pre-determined value.

According to another aspect, there is provided a method for monitoring failure of a tissue on a patient may comprise of the steps:
(i) providing a first sensor on or within said tissue, said first sensor being capable of detecting and measuring the amount of a first metabolite;
(ii) providing a second sensor on a control region of said patient, said control region being separate from said tissue and wherein said second sensor is capable of detecting and measuring the amount of said first metabolite;
(iii) providing a third sensor on or within said tissue, said third sensor being capable of detecting and measuring the amount of a second metabolite and wherein said third sensor is the same as or different from the first sensor;
(iv) providing a fourth sensor on said control region of said patient, said fourth sensor being capable of detecting and measuring the amount of said second metabolite and wherein said fourth sensor is the same as or different from the second sensor;
(v) monitoring the amounts of said first metabolite measured by both said first and second sensors for a period of time;
(vi) monitoring the amounts of said second metabolite measured by both said third and fourth sensors for a period of time;

wherein an at least 10% decrease in the amount of said first metabolite as measured by said first sensor as compared to the amount of said first metabolite as measured by said second sensor; and at least 10% increase in the amount of said second metabolite as measured by said third sensor as compared to the amount of said second metabolite as measured by said fourth sensor, is indicative that said tissue is prone to failure.

According to another aspect, there is provided a method for monitoring failure of a tissue on a patient, comprising the steps of:
(i) providing a first sensor on or within said tissue, said first sensor being capable of detecting and measuring the amount of a first metabolite;
(ii) providing a second sensor on a control region of said patient, said control region being separate from said tissue and wherein said second sensor is capable of detecting and measuring the amount of said first metabolite;
(iii) providing a third sensor on or within said tissue, said third sensor being capable of detecting and measuring the amount of a second metabolite and wherein said third sensor is the same as or different from the first sensor;
(iv) providing a fourth sensor on said control region of said patient, said fourth sensor being capable of detecting and measuring the amount of said second metabolite and wherein said fourth sensor is the same as or different from the second sensor;
(v) monitoring the amounts of said first metabolite measured by both said first and second sensors for a period of time;
(vi) monitoring the amounts of said second metabolite measured by both said third and fourth sensors for a period of time;

wherein the amount of said first metabolite as measured by said first sensor and the amount of said first metabolite as measured by said second sensor are substantially the same; and at least 10% increase in the amount of said second metabolite as measured by said third sensor as compared to the amount of said second metabolite as measured by said fourth sensor, is indicative that said tissue is prone to failure.

According to another aspect, there is provided a method for monitoring failure of a tissue on a patient, comprising the steps of:
(i) providing a first sensor on or within said tissue, said first sensor being capable of detecting and measuring the amount of a first metabolite;
(ii) providing a second sensor on a control region of said patient, said control region being separate from said tissue and wherein said second sensor is capable of detecting and measuring the amount of said first metabolite;
(iii) providing a third sensor on or within said tissue, said third sensor being capable of detecting and measuring the amount of a second metabolite and wherein said third sensor is the same as or different from the first sensor;
(iv) providing a fourth sensor on said control region of said patient, said fourth sensor being capable of detecting and measuring the amount of said second metabolite and wherein said fourth sensor is the same as or different from the second sensor;
(v) monitoring the amounts of said first metabolite measured by both said first and second sensors for a period of time;
(vi) monitoring the amounts of said second metabolite measured by both said third and fourth sensors for a period of time;

wherein an at least 10% decrease in the amount of said first metabolite as measured by said first sensor as compared to the amount of said first metabolite as measured by said second sensor; and the amount of said second metabolite as measured by said third sensor and the amount of said second metabolite as measured by said fourth sensor are substantially the same, is indicative that said tissue is prone to failure.

According to another aspect, there is provided a method for monitoring failure of a tissue on a patient, comprising the steps of:
(i) providing a first sensor on or within said tissue, said first sensor being capable of detecting and measuring the amount of a first metabolite;
(ii) providing a second sensor on a control region of said patient, said control region being separate from said tissue and wherein said second sensor is capable of detecting and measuring the amount of said first metabolite;
(iii) providing a third sensor on or within said tissue, said third sensor being capable of detecting and measuring the amount of a second metabolite and wherein said third sensor is the same as or different from the first sensor;
(iv) providing a fourth sensor on said control region of said patient, said fourth sensor being capable of detecting and measuring the amount of said second metabolite and wherein said fourth sensor is the same as or different from the second sensor;
(v) monitoring the amounts of said first metabolite measured by both said first and second sensors for a period of time;
(vi) monitoring the amounts of said second metabolite measured by both said third and fourth sensors for a period of time;

wherein an at least 10% decrease in the amount of said first metabolite as measured by said first sensor as compared to the amount of said first metabolite as measured by said second sensor; and an at least 10% decrease in the amount of said second metabolite as measured by said third sensor as compared to the amount of said second metabolite as measured by said fourth sensor, is indicative that said tissue is prone to failure.

According to another aspect, there is provided a method for monitoring failure of a tissue on a patient, comprising the steps of:
(i) providing a first sensor on or within said tissue, said first sensor being capable of detecting and measuring the amount of a first metabolite;
(ii) providing a second sensor on a control region of said patient, said control region being separate from said tissue and wherein said second sensor is capable of detecting and measuring the amount of said first metabolite;
(iii) providing a third sensor on or within said tissue, said third sensor being capable of detecting and measuring the amount of a second metabolite and wherein said third sensor is the same as or different from the first sensor;
(iv) providing a fourth sensor on said control region of said patient, said fourth sensor being capable of detecting and measuring the amount of said second metabolite and wherein said fourth sensor is the same as or different from the second sensor;
(v) monitoring the amounts of said first metabolite measured by both said first and second sensors for a period of time;
(vi) monitoring the amounts of said second metabolite measured by both said third and fourth sensors for a period of time;

wherein an at least 10% increase in the amount of said first metabolite as measured by said first sensor as compared to the amount of said first metabolite as measured by said second sensor; and an at least 10% increase in the amount of said second metabolite as measured by said third sensor as compared to the amount of said second metabolite as measured by said fourth sensor, is indicative that said tissue is prone to failure.

Advantageously, the monitor comprising the sensors may be highly specific with high accuracy. Further advantageously, the monitor comprising the sensors may prevent false alarms from being raised.

### Definitions

In this specification a number of terms are used which are well known to a skilled addressee.

Nevertheless for the purposes of clarity a number of terms will be defined. The following words and terms used herein shall have the meaning indicated:

The term “redox polymer” is used interchangeably with the term “electrochemical activator”, which when used herein refers to any compound that is capable of activating the enzyme that transfers electrons between the enzyme (glucose oxidase/lactate oxidase) and the working (detection) electrode of the sensor. This can also be known as an electron transfer agent. The glucose oxidase may be abbreviated as GOx. The lactate oxidase may be abbreviated as LOx.

The term “ferrocenyl derivative” is used interchangeably with the term “ferrocene derivative”, and refers to a derivative containing an optionally substituted ferrocene or ferrocenyl.

The term “enzyme” is used interchangeably with the term “oxidoreductase”, which refers to the ability to catalyse the oxidation or reduction of a substrate or an analyte by the removal or addition of electrons. The term “oxidoreductase enzyme” may also be used interchangeably.

The term “order” when used herein refers to the arrangement or disposition of the compound monomers in relation to each other according to a particular sequence, pattern or method.

The term “non-order” when used herein refers to the opposite of the above definition wherein the arrangement or disposition of the compound monomers is of no particular sequence, pattern or method or in a random manner.

The term “random” when used herein refers to something that is unpredictable or lacking uniformity when being arranged or without regularity.

The term “tissue” when used herein can be defined broadly to refer to a flap, a membrane, skin, meninges, connective tissue, or organs such as liver, stomach, pancreas, intestine, kidney, thymus, uterus, testes, bladder, lung, heart. The term “tissue” may also encompass free tissue (such as a tissue that is or was isolated from an animal or human subject).

The term “failure” when used herein refers to tissue with compromised blood supply.

In the definitions of a number of substituents below it is stated that “the group may be a terminal group or a bridging group”. This is intended to signify that the use of the term is intended to encompass the situation where the group is a linker between two other portions of the molecule as well as where it is a terminal moiety. Using the term alkyl as an example, some publications would use the term “alkylene” for a bridging group and hence in these other publications there is a distinction between the terms “alkyl” (terminal group) and “alkylene” (bridging group). In the present application no such distinction is made and most groups may be either a bridging group or a terminal group.

“Acyl” refers to an R—C(═O)— group in which the R group may be an optionally substituted alkyl, optionally substituted cycloalkyl, optionally substituted heterocycloalkyl, optionally substituted aryl or optionally substituted heteroaryl group as defined herein. Examples of acyl include acetyl. The group may be a terminal group or a bridging group. If the group is a terminal group it is bonded to the remainder of the molecule through the carbonyl carbon.

“Acyloxy” refers to an R—C(═O)—O— group in which the R group may be an alkyl, cycloalkyl, heterocycloalkyl; aryl or heteroaryl group as defined herein. The group may be a terminal group or a bridging group. If the group is a terminal group it is bonded to the remainder of the molecule through the oxygen atom.

“Alkanoyl” refers to an alkyl-C═O group in which alkyl is as defined herein. Preferred alkanoyl groups are C₁-C₆ alkanoyl groups. The group may be a terminal group or a bridging group. If the group is a terminal group it is bonded to the remainder of the molecule through the carbon atom of the carbonyl group.

“Alkyl” as a group or part of a group refers to a straight or branched aliphatic hydrocarbon group, preferably a C₁-C₁₂ alkyl, more preferably a C₁-C₁₀ alkyl, most preferably C₁-C₆ unless otherwise noted. Examples of suitable straight and branched C₁-C₆ alkyl substituents include methyl, ethyl, n-propyl, 2-propyl, n-butyl, sec-butyl, t-butyl, hexyl, and the like. The group may be a terminal group or a bridging group.

“Alkylamino” includes both mono-alkylamino and dialkylamino, unless specified. “Mono-alkylamino” means a alkyl-NH— group, in which alkyl is as defined herein. “Dialkylamino” means a (alkyl)₂N— group, in which each alkyl may be the same or different and are each as defined herein for alkyl. The alkyl group is preferably a C₁-C₆ alkyl group. The group may be a terminal group or a bridging group. If the group is a terminal group it is bonded to the remainder of the molecule through the nitrogen atom.

“Alkylaminoalkyl” refers to an alkyl-N-alkyl group, in which each alkyl may be the same or different and are each as defined herein for alkyl. The alkyl group is preferably a C₁-C₆ alkyl group. The group may be a terminal group or a bridging group. If the group is a terminal group it is bonded to the remainder of the molecule through the nitrogen atom.

“Alkyloxy” refers to an alkyl-O— group in which alkyl is as defined herein. Preferably the alkyloxy is a C₁-C₆alkyloxy. Examples include, but are not limited to, methoxy and ethoxy. The group may be a terminal group or a bridging group. The term alkyloxy may be used interchangeably with the term “alkoxy”.

“Alkyloxyalkyl” refers to an alkyloxy-alkyl- group in which the alkyloxy and alkyl moieties are as defined herein. The group may be a terminal group or a bridging group. If the group is a terminal group it is bonded to the remainder of the molecule through the alkyl group.

“Alkylthio” refers to an alkyl-S— group in which alkyl is as defined herein. Preferably the alkylthio is a C₁-C₆alkylthio. Examples include, but are not limited to, dimethylsulfide ((CH₃)₂S) and diethylsulfide. The group may be a terminal group or a bridging group.

“Alkylthioalkyl” refers to an alkylthio-alkyl- group in which the alkylthio and alkyl moieties are as defined herein. The term “alkylthioalkyl” may also refer to a thioether in which the term is —C—S—C— and may be used interchangeably. The group may be a terminal group or a bridging group. If the group is a terminal group it is bonded to the remainder of the molecule through the alkyl group.

“Amino” refers to groups of the form NRₐR_{b} wherein Rₐ and R_{b} are individually selected from the group including but not limited to hydrogen, optionally substituted alkyl, optionally substituted alkenyl, optionally substituted alkynyl, and optionally substituted aryl groups.

“Aminoalkyl” means an NH₂-alkyl- group in which the alkyl group is as defined herein. The group may be a terminal group or a bridging group. If the group is a terminal group it is bonded to the remainder of the molecule through the alkyl group. The “alkylamine” may be interchangeably used.

“Hydroxyalkyl” refers to an alkyl group as defined herein in which one or more of the hydrogen atoms has been replaced with an OH group. A hydroxyalkyl group typically has the formula CₙH₍₂ₙ₊₁₋ₓ₎(OH) In groups of this type n is typically from 1 to 10, more preferably from 1 to 6, most preferably from 1 to 3. x is typically from 1 to 6, more preferably from 1 to 4.

It is understood that included in the family of compounds of Formula (I), may have isomeric forms including diastereoisomers, enantiomers, tautomers, and geometrical isomers in “E” or “Z” configurational isomer or a mixture of E and Z isomers. It is also understood that some of the isomeric forms including planar chirality compounds in “R” or “S” configuration. It is also understood that some isomeric forms such as diastereomers, enantiomers, and geometrical isomers can be separated by physical and/or chemical methods and by those skilled in the art.

Some of the compounds of the disclosed embodiments may exist as single stereoisomers, racemates, and/or mixtures of enantiomers and/or diastereomers. All such single stereoisomers, racemates and mixtures thereof, are intended to be within the scope of the subject matter described and claimed.

Further, it is possible that compounds of the invention may contain more than one asymmetric carbon atom. In those compounds, the use of a solid line to depict bonds to asymmetric carbon atoms is meant to indicate that all possible stereoisomers are meant to be included. The use of a solid line to depict bonds to one or more asymmetric carbon atoms in a compound of the invention and the use of a solid or dotted wedge to depict bonds to other asymmetric carbon atoms in the same compound is meant to indicate that a mixture of diastereomers is present.

The term “optionally substituted” as used herein means the group to which this term refers may be unsubstituted, or may be substituted with one or more groups independently selected from alkyl, alkenyl, alkynyl, thioalkyl, cycloalkyl, cycloalkylalkyl, cycloalkenyl, cycloalkylalkenyl, heterocycloalkyl, cycloalkylheteroalkyl, cycloalkyloxy, cycloalkenyloxy, cycloamino, halo, carboxyl, haloalkyl, haloalkynyl, alkynyloxy, heteroalkyl, heteroalkyloxy, hydroxyl, hydroxyalkyl, alkoxy, thioalkoxy, alkenyloxy, haloalkoxy, haloalkenyl, haloalkynyl, haloalkenyloxy, nitro, amino, nitroalkyl, nitroalkenyl, nitroalkynyl, nitroheterocyclyl, alkylamino, dialkylamino, alkenylamine, aminoalkyl, alkynylamino, acyl, alkyloxy, alkyloxyalkyl, alkyloxyaryl, alkyloxycarbonyl, alkyloxycycloalkyl, alkyloxyheteroaryl, alkyloxyheterocycloalkyl, alkenoyl, alkynoyl, acylamino, diacylamino, acyloxy, alkylsulfonyloxy, heterocyclic, heterocycloalkenyl, heterocycloalkyl, heterocycloalkylalkyl, heterocycloalkylalkenyl, heterocycloalkylheteroalkyl, heterocycloalkyloxy, heterocycloalkenyloxy, heterocycloxy, heterocycloamino, haloheterocycloalkyl, alkylsulfinyl, alkylsulfonyl, alkylsulfenyl, alkylcarbonyloxy, alkylthio, acylthio, aminosulfonyl, phosphorus-containing groups such as phosphono and phosphinyl, sulfinyl, sulfinylamino, sulfonyl, sulfonylamino, aryl, heteroaryl, heteroarylalkyl, heteroarylalkenyl, heteroarylheteroalkyl, heteroarylamino, heteroaryloxy, arylalkenyl, arylalkyl, alkylaryl, alkylheteroaryl, aryloxy, arylsulfonyl, cyano, cyanate, isocyanate, —C(O)NH(alkyl), and —C(O)N(alkyl)₂.

Preferably, the alkyl is an optionally substituted C₁-C₆ alkyl, alkylamino is an optionally substituted alkyl-NH— group having a C₁-C₆ alkyl group, dialkylamino is an optionally substituted (alkyl)₂N— group having a C₁-C₆ alkyl group, alkylaminoalkyl is an optionally substituted alkyl-NH-alkyl group having a C₁-C₆ alkyl group, alkyloxycarbonyl is a an optionally substituted C₁-C₁₆ alkyloxy having a carbonyl group, alkanoyl is an optionally substituted C₁-C₆ alkyl having a carbonyl group.

The term “lower alkyl” as used herein whether employed as an independent substituent or as a part of another substituent includes straight or branched chain aliphatic hydrocarbon radicals having up to and including 7 carbon atoms. Examples of lower alkyls include, but are not limited to, alkyl groups having 1 to 3 carbons, such as methyl, ethyl, propyl, and isopropyl and alkyl groups having 4 to 7 carbons, such as butyl, isobutyl, t-butyl, amyl, hexyl, heptyl and the like.

The word “substantially” does not exclude “completely” e.g. a composition which is “substantially free” from Y may be completely free from Y. Where necessary, the word “substantially” may be omitted from the definition of the invention.

Unless specified otherwise, the terms “comprising” and “comprise”, and grammatical variants thereof, are intended to represent “open” or “inclusive” language such that they include recited elements but also permit inclusion of additional, unrecited elements.

As used herein, the term “about”, in the context of concentrations of components of the formulations, typically means±10% of the stated value, more typically ±7.5% of the stated value, more typically ±5% of the stated value, more typically ±4% of the stated value, more typically ±3% of the stated value, more typically, ±2% of the stated value, even more typically ±1% of the stated value, and even more typically ±0.5% of the stated value.

Throughout this disclosure, certain embodiments may be disclosed in a range format. It should be understood that the description in range format is merely for convenience and brevity and should not be construed as an inflexible limitation on the scope of the disclosed ranges. Accordingly, the description of a range should be considered to have specifically disclosed all the possible sub-ranges as well as individual numerical values within that range. For example, description of a range such as from 1 to 6 should be considered to have specifically disclosed sub-ranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Certain embodiments may also be described broadly and generically herein. Each of the narrower species and subgeneric groupings falling within the generic disclosure also form part of the disclosure. This includes the generic description of the embodiments with a proviso or negative limitation removing any subject matter from the genus, regardless of whether or not the excised material is specifically recited herein.

### DETAILED DESCRIPTION OF EMBODIMENTS

Provided herein are enzyme-containing polymers useful for highly selective real time detection of analytes under various conditions, including under physiological conditions. Advantageously, the enzyme-containing polymers described herein may be stable up to five (5) days or more of continuous usage and the shelf-life of the enzyme-containing polymer may be up to six (6) months or more at recommended storage conditions. Moreover, the enzyme-containing polymers described herein generate strong enough signals for use with protective layer which tends to attenuate the final electron signal. Exemplary, non-limiting embodiments of the enzyme-containing polymer will now be disclosed.

In certain embodiments, the enzyme-containing polymer comprises:

a first repeating of Formula Ia:

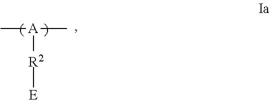

a second repeating unit of Formula Ib:

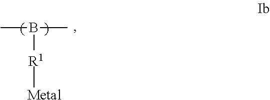

and

a third repeating unit of Formula Ic:

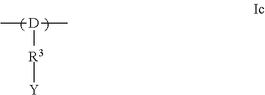

or conjugate salts thereof, wherein

each of A, B, and D is independently a 2-amino monosaccharide;

E is an enzyme comprising an n-terminal amine and optionally one or more lysine residues, wherein R² is covalently bonded to the n-terminal amine or the amine side chain of the one or more lysine residues;

Metal is a metal complex;

R¹ is —N*(R)(C═O)—, —N*(R)(C═O)N(R)—, —N*(R)(CR₂)ₙ—, —N*(R)(CR₂)ₙ(C═O)—, —N*(R)(C═O)(CR₂)ₙ—, —N*(R)(C═O)(CR₂)ₙ(C═O)—, —N*(R)(CR₂)ₙO(CR₂)ₘ—, —N*(R)(CR₂)ₙS(CR₂)ₘ—, —N*(R)(CR₂)ₙO—, —N*(R)(CR₂)ₙ(C═O)O—, —N*(R)(C═O)(CR₂)ₙO—, —N*(R)(C═O)(CR₂)ₙ(C═O)O—, —N*(R)(CR₂)ₙO(CR₂)ₘO—, —N*(R)(CR₂)ₙS(CR₂)ₘO—, —N*═CH(CR₂)ₙ—, —N*═CH(CR₂)ₙ(C═O)—, —N*═CH(CR₂)ₙO(CR₂)ₘ—, —N*═CH(CR₂)ₙS(CR₂)ₘ—, —N*═CH(CR₂)ₙO—, —N*═CH(CR₂)ₙ(C═O)O—, —N*═CH(CR₂)ₙO(CR₂)ₘO—, or —N*═CH(CR₂)ₙS(CR₂)ₘO—, wherein N* represents the nitrogen at the 2 position of the 2-amino monosaccharide;

R² is —N*(R)(CR₂)ₙN**(R)—, —N*(R)(CR₂)ₙ(C═O)N**(R)—, —N*(R)(C═O)(CR₂)ₙ—, —N*(R)(C═O)(CR₂)ₙ(C═O)N**(R)—, —N*(R)(CR₂)ₙO(CR₂)ₘN**(R)—, —N*(R)(CR₂)ₙS(CR₂)ₘN**(R)—, —N*═CH(CR₂)ₙN**(R)—, —N*═CH(CR₂)ₙ(C═O)N**(R)—, —N*═CH(CR₂)ₙO(CR₂)ₘN**(R)—, —N*═CH(CR₂)ₙS(CR₂)ₘN**(R)—, N*(R)(CR₂)ₙCH═N**—, —N*(R)(C═O)(CR₂)ₙCH═N**—, —N*(R)(CR₂)ₙO(CR₂)ₘCH═N**—, —N*(R)(CR₂)ₙS(CR₂)ₘCH═N**—, —N*═CH(CR₂)ₙCH═N**—, —N*═CH(CR₂)ₙO(CR₂)ₘCH═N**—, or —N*═CH(CR₂)ₙS(CR₂)ₘCH═N**—, wherein N* represents the nitrogen at the 2 position of the 2-amino monosaccharide and N** represents the nitrogen of the n-terminal amine or the amine side chain of the one or more lysine residues of the enzyme; or R² is represented by the moiety:

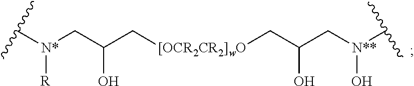

R³ is —N*(R)(CR₂)ₙY, —N*(R)(CR₂)ₙ(C═O)Y, —N*(R)(C═O)(CR₂)ₙY, —N*(R)(C═O)(CR₂)ₙ(C═O)Y, —N*(R)(CR₂)ₙO(CR₂)ₘY, —N*(R)(CR₂)ₙS(CR₂)ₘY, —N*═CH(CR₂)ₙY, —N*═CH(CR₂)ₙ(C═O)Y, —N*═CH(CR₂)ₙO(CR₂)ₘY, —N*═CH(CR₂)ₙS(CR₂)ₘY, —N*(R)(CR₂)ₙCH═Y, —N*(R)(C═O)(CR₂)ₙCH═Y, —N*(R)(CR₂)ₙO(CH₂)ₘCH═Y, - —N*(R)(CR₂)ₙS(CR₂)ₘCH═Y, —N*═CH(CR₂)ₙCH═Y, —N*═CH(CR₂)ₙO(CR₂)ₘCH═Y, or —N*═CH(CR₂)ₙS(CR₂)ₘCH═Y, wherein N* represents the nitrogen at the 2 position of the 2-amino monosaccharide; or R³ is represented by the moiety:

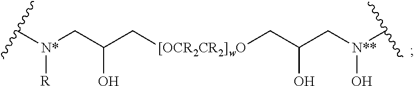

R for each occurrence is independently hydrogen, lower alkyl or hydroxyl;

m for each occurrence is independently a whole number selected between 1-20;

n for each occurrence is independently a whole number selected between 1-20;

w for each occurrence is independently a whole number selected between 1-20; and

Y is a polyalkylamine comprising at least one metal complex, wherein the polyalkylamine optionally crosslinks at least two of the third repeating units. The metal complex can be any substantially non-toxic metal complex known in the art. The selection of the metal complex is within the skill of a person of ordinary skill in the art. In certain embodiments, the metal complex has a redox potential lower than hydrogen peroxide under physiological conditions. Exemplary metal complexes include, but are not limited to iron or ferrocenyl complexes, copper complexes, cobaltocenium complexes, ruthenium complexes, osmium complexes, zinc complexes or combinations thereof. In certain embodiments, the metal complex is optionally substituted ferrocenyl.

In the enzyme-containing polymer, the metal may be an optionally substituted ferrocenyl derivative. The ferrocenyl derivative may be taken to be any ferrocenyl derivative containing the ferrocene and other functional moieties. The ferrocenyl derivative may be optionally substituted with other functional moieties. The ferrocenyl derivative may be represented by a formula Fc:

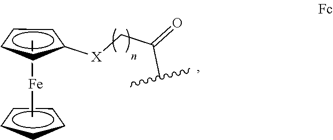

wherein X may independently represent at least an alkyl group, a carbonyl group or may be absent, and n may independently be an integer from 0 to 10 or 0, 1, 2, 3, 4, 5, 6, 7, 8, 9 or 10.

The ferrocenyl derivative may be selected from the group consisting of:

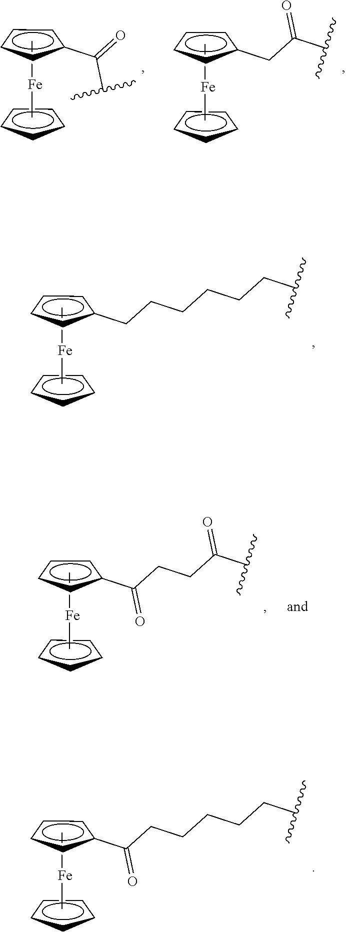

The optionally substituted ferrocenyl derivative may be attached to the enzyme-containing polymer via the R¹ substituent with a grafting percentage (ratio) in the range of 30% w/w to 50% w/w, 35% w/w to 50% w/w, 40% w/w to 50% w/w, 45% w/w to 50% w/w, 30% w/w to 35% w/w, 30% w/w to 40% w/w or 30% w/w to 45% w/w. In certain embodiments, the optionally substituted ferrocenyl derivative is attached to the enzyme-containing polymer via the R¹ substituent with a grafting ratio of 40% w/w.

The ferrocenyl derivative may be able to provide localized electroactivity and thus the ability to take part in redox reactions. In certain embodiments, the ferrocenyl derivative does not bring about reorganization of the bonds in the polymer when it takes part in redox reactions.

The molar ratio of the first repeating unit to the third repeating unit in the enzyme-containing polymer may be between 1:5 to 5:1. In certain embodiments, the molar ratio of the first repeating unit to the third repeating unit in the enzyme-containing polymer is between 1:4 to 4:1; 1:3 to 3:1; 1:2 to 2:1; or 0.8:1 to 1:0.8. In certain embodiments, the molar ratio of the first repeating unit to the second repeating unit in the enzyme-containing polymer is between 3:1, 2:1, 1:1, 1:2 or 1:3.

Each of A, B, and D can independently be any 2-amino monosaccharide known in the art. Exemplary 2-amino monosaccharides include, but are not limited to, 2-amino-2-deoxy-(D or L)-arabinose, 3-deoxy-3-(methylamino)-L-arabinose (4-epi-gentosamine), 2-amino-2-deoxy-D-ribose, 2-amino-2-deoxypentofuranose, 2-amino-2-deoxy-D-xylose, 2-amino-2-deoxy-D-allopyranose (D-allosamine), 2-amino-2-deoxy-D-galactose (chondrosamine, D-galactosamine), 2,6-dideoxy-2-(methylamino)-D-galactose (methylfucosamine), 2-amino-2-deoxy-D-glucose (D-glucosamine, chitosamine), 2-amino-2-deoxy-L-glucose, 2-amino-2-deoxymannose (mannosamine), and 2-deoxy-2-(methylamino)-L-glucose. In certain embodiments, 2-amino monosaccharide is selected from the group consisting of glucosamine, mannosamine, and galactosamine. In certain embodiments, 2-amino monosaccharide is glucosamine.

Advantageously, the enzyme-containing polymer may be stable, biocompatible and biodegradable.

In certain embodiments, the enzyme-containing polymer comprises a modified chitosan polymer, wherein the glucosamine monomer units of the chitosan are synthetically modified thereby forming the first repeating unit, the second repeating unit, and the third repeating unit. In such embodiments, the modified chitosan polymer may further comprise monomer units selected from the group consisting of N-acetylglucosamine, glucosamine, and combinations thereof.

In instances in which the enzyme-containing polymer is prepared from chitosan, the chitosan may have a molecular weight (M_{w}) of between about 10,000 g/mol to about 250,000 g/mol, about 20,000 g/mol to about 250,000 g/mol, about 30,000 g/mol to about 250,000 g/mol, about 40,000 g/mol to about 250,000 g/mol, about 50,000 g/mol to about 250,000 g/mol, about 60,000 g/mol to about 250,000 g/mol, about 70,000 g/mol to about 250,000 g/mol, about 80,000 g/mol to about 250,000 g/mol, about 90,000 g/mol to about 250,000 g/mol, about 100,000 g/mol to about 250,000 g/mol, about 120,000 g/mol to about 250,000 g/mol, about 140,000 g/mol to about 250,000 g/mol, about 160,000 g/mol to about 250,000 g/mol, about 180,000 g/mol to about 250,000 g/mol, about 200,000 g/mol to about 250,000 g/mol, about 220,000 g/mol to about 250,000 g/mol, about 240,000 g/mol to about 250,000 g/mol, about 10,000 g/mol to about 240,000 g/mol, about 10,000 g/mol to about 220,000 g/mol, about 10,000 g/mol to about 200,000 g/mol, about 10,000 g/mol to about 180,000 g/mol, about 10,000 g/mol to about 160,000 g/mol, about 10,000 g/mol to about 140,000 g/mol, about 10,000 g/mol to about 120,000 g/mol, about 10,000 g/mol to about 100,000 g/mol, about 10,000 g/mol to about 90,000 g/mol, about 10,000 g/mol to about 80,000 g/mol, about 10,000 g/mol to about 70,000 g/mol, about 10,000 g/mol to about 60,000 g/mol, about 10,000 g/mol to about 50,000 g/mol, about 10,000 g/mol to about 40,000 g/mol, about 10,000 g/mol to about 30,000 g/mol or about 10,000 g/mol to about 20,000 g/mol. The chitosan monomer units may have a deacetylation degree (DD) of more than 75%, or 80% or greater. In certain embodiments, the DD of the chitosan is between 75% to 95%, 75% to 85% or 85% to 95%.

The enzyme-containing polymer may comprise between 1 and 1,500 of each of the first repeating unit, the second repeating unit, and the third repeating unit. In certain embodiments, enzyme-containing polymer comprises between 1 to 500 or 1 to 50 of each of the first repeating unit, the second repeating unit, and the third repeating unit. The enzyme-containing polymer may be a random, alternating, block or sequential copolymer comprising the first repeating unit, the second repeating unit, and the third repeating unit.

In certain embodiments, the enzyme is selected from the group consisting of glucose oxidase, lactate oxidase, xanthine oxidase, cholesterol oxidase, malate oxidase, galactose oxidase, xanthine dehydrogenase, glucose dehydrogenase, lactate dehydrogenase, alcohol oxidase, choline oxidase, xanthine oxidase, glutamate oxidase or amine oxidase. In certain embodiments, the enzyme is glucose oxidase or lactate oxidase. In certain embodiments, the enzyme is glucose oxidase (Aspergillus niger) or lactate oxidase (Aerococcus viridans).

The enzyme may help to catalyse the oxidation or reduction of the substrate by the removal or addition of electrons. The enzyme may be glucose oxidase that is used to detect the presence or the concentration of glucose from an analyte (when the sensor containing the enzyme-containing polymer is placed at a testing site in or on a patient). The glucose oxidase may be used to oxidize glucose to gluconic acid. The presence of a suitable oxidoreductase may help to accelerate the oxidation reaction, hence allowing the enzyme activity and analyte analysis to be easily investigated. The enzyme may also be lactate oxidase to detect lactate in an analyte. The lactate oxidase may be used to catalyse the oxidation of lactate to pyruvate in the presence of oxygen.

Advantageously, the specific enzyme of the enzyme-containing polymer may bind to a selected metabolite only and the redox polymer which is a mediator would then allow for improved electron transfer from the enzyme to the electrode, which in turn leads to a better signal being received at the electrode.

When a specific enzyme is selected from a large variety of enzymes and employed in a sensor, a specific metabolite according to the specific enzyme may be detected from an analyte. The specific metabolite may be from a large variety of metabolites. The metabolite may be a first metabolite and/or a second metabolite. The number of metabolites detected from analytes may be at least two metabolites. The first and/or second metabolite may be the same or different metabolite. The first and/or second metabolite may be selected from the group consisting of glucose, lactate, xanthine, cholesterol, malate, galactose, xanthine, alcohol, choline, xanthine, glutamate and amine Where applicable, additional metabolites in addition to the first and second metabolites may be detected as well. In certain embodiments, R¹ is —N*(R)(C═O)—, —N*(R)(CR₂)ₙ—, —N*(R)(CR₂)ₙ(C═O)—, —N*(R)(C═O)(CR₂)ₙ—, —N*(R)(C═O)(CR₂)ₙ(C═O)—, —N*(R)(CR₂)ₙO—, —N*(R)(CR₂)ₙ(C═O)O—, —N*(R)(C═O)(CR₂)ₙO—, or —N*(R)(C═O)(CR₂)ₙ(C═O)O—. In certain embodiments, R¹ is —N*(R)(C═O)—, —N*(R)(C═O)(CR₂)ₙ—, or —N*(R)(C═O)(CR₂)ₙ(C═O)—, or —N*(R)(C═O)(CR₂)ₙO—, wherein n is a whole number selected between 1-10; 1-8; or 1-6; and m is a whole number selected between 1-10; 1-8; or 1-6. In certain embodiments, R¹ is —N*(H)(C═O)—, —N*(H)(C═O)(CH₂)ₙ—, —N*(H)(C═O)(CH₂)ₙ(C═O)—, or —N*(H)(C═O)(CH₂)ₙO—, and n is a whole number selected between 1-10; 1-8; or 1-6. In certain embodiments, R¹ is —N*(H)(CH₂)₆—, —N*(H)(CH₂)₅(C═O)—, —N*(H)(C═O)—, —N*(H)(CH₂)(C═O)—, —N*(H)(C═O)(CH₂)₂(C═O)—.

In the enzyme-containing polymer, R¹ may represent an optionally substituted alkyl, an optionally substituted alkanoyl, an optionally substituted alkylaminoalkyl, an optionally substituted alkylthioalkyl or an optionally substituted alkyloxyalkyl divalent linker. R¹ may be selected from the group consisting of an optionally substituted C₂-C₁₈ alkyl, an optionally substituted C₂-C₁₈ alkanoyl, an optionally substituted C₂-C₁₈ alkylaminoalkyl, an optionally substituted C₂-C₁₈ alkylthioalkyl, and an optionally substituted C₂-C₁₈ alkyloxyalkyl. R¹ may be an optionally substituted C₂-C₁₂ alkyl or an optionally substituted C₂-C₈ alkyl divalent linker.

In certain embodiments, R² is —N*═CH(CR₂)ₙCH═N**—. In certain embodiments, R² is —N*═CH(CR₂)ₙCH═N**—, wherein n is 1-10; 1-8; 1-6. In certain embodiments, R² is —N*═CH(CH₂)₄CH═N**—.

In certain embodiments, R² is represented by the moiety:

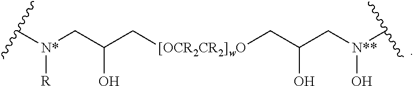

In certain embodiments, R³ is —N*═CH(CR₂)ₙCH═N**—. In certain embodiments, R² is —N*═CH(CR₂)ₙCH═N**—, wherein n is 1-10; 1-8; 1-6. In certain embodiments, R² is —N*═CH(CH₂)₄CH═N**—.

In certain embodiments, R³ is represented by the moiety:

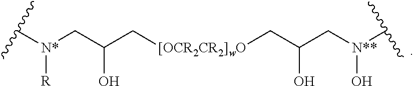

In certain embodiments, R is hydrogen, C₁-C₆ alkyl, C₁-C₅ alkyl, C₁-C₄ alkyl, C₁-C₃ alkyl, or C₁-C₂ alkyl. In certain embodiments, R is hydrogen or methyl. In certain embodiments, R is hydrogen.

In certain embodiments, m for each occurrence is independently a whole number selected between 1-20; 1-16; 1-14; 1-12; 1-10; 1-8; 1-7; 1-6; 1-5; 1-4; 2-8; or 2-6.

In certain embodiments, n for each occurrence is independently a whole number selected between 1-20; 1-16; 1-14; 1-12; 1-10; 1-8; 1-7; 1-6; 1-5; 1-4; 2-8; or 2-6.

In certain embodiments, w for each occurrence is independently a whole number selected between 1-20; 1-16; 1-14; 1-12; 1-10; 1-8; 1-7; 1-6; 1-5; 1-4; 2-8; or 2-6.

Y can be any polyalkylamine known in the art. The polyalkylamine may comprise ethylenediamine repeating units. The polyalkylamine may be linear or branched, or dendritic. The amino groups of the polyalkylamine may be selected from primary, secondary or tertiary amino groups, or combinations thereof. The polyalkylamine may be selected from polyethyleneimine and poly(propylene imine) The polyalkylamine may preferably be branched polyethyleneimine.

In certain embodiments, Y is a polyethylenimine selected from the group consisting of linear polyethylenimine, branched polyethylenimine, and dendritic polyethylenimine. The polyethylenimine can have an average molecular weight of between 1,000 to 50,000 amu. In certain embodiments, the polyethylenimine has an average molecular weight of between 5,000 to 50,000; 5,000 to 40,000; 5,000 to 30,000; 10,000 to 30,000; 15,000 to 25,000 amu. In certain embodiments, the polyethylenimine is branched and has an average molecular weight of 5,000 to 25,000; 10,000 to 25,000; 15,000 to 25,000; 17,000 to 25,000; 17,000 to 22,000; 18,000 to 22,000; or 19,000 to 22,000 amu.

The polyalkylamine can comprise at least one metal complex. The metal complex can be any substantially non-toxic metal complex known in the art. The selection of the metal complex is within the skill of a person of ordinary skill in the art. In certain embodiments, the metal is optionally substituted ferrocenyl.

The optionally substituted ferrocenyl derivative may be attached to the polyalkylamine with a grafting percentage (ratio) in the range of 30% w/w to 50% w/w, 35% w/w to 50% w/w, 40% w/w to 50% w/w, 45% w/w to 50% w/w, 30% w/w to 35% w/w, 30% w/w to 40% w/w or 30% w/w to 45% w/w. In certain embodiments, the optionally substituted ferrocenyl derivative is attached to the polyalkylamine with a grafting ratio of 40% w/w.

Branched polyethylenimines can have a highly irregular structure comprising secondary amines, tertiary amines and primary amine end groups. An exemplary simplified representation of just one of many possible branched polyethylenimine repeating units is depicted below:

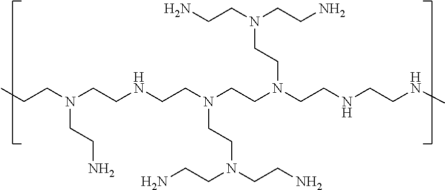

Numerous structural variations of the aforementioned exemplary branched polyethylenimine repeating unit can be present in the branched polyethylenimine.

The branched polyethylenimine can comprise one or more metal complexes, such as an optionally substituted ferrocenyl. The metal complexes can be covalently bonded to any one or more of the primary or secondary amines as indicated by the arrows below:

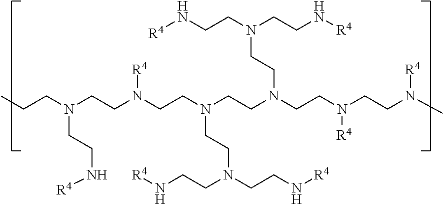

wherein R⁴ for each instance is independently hydrogen or a metal complex (such as optionally substituted ferrocenyl) with the proviso that at least one R⁴ is a metal complex. The metal complex can optionally be attached to the polyalkylamine (e.g., branched polyethylenimine) via an optional linker. The linker can be selected from the group consisting of —N‡(R^{X})(C═O)—, —N‡(R^{X})(CR₂)ₙ—, —N‡(R^{X})(CR₂)ₙ(C═O)—, —N‡(R^{X})(C═O)(CR₂)ₙ—, —N‡(R^{X})(C═O)(CR₂)ₙ(C═O)—, —N‡(R^{X})(CR₂)ₙO—, —N‡(R^{X})(CR₂)ₙ(C═O)O—, —N‡(R^{X})(C═O)(CR₂)ₙO—, or —N‡(R^{X})(C═O)(CR₂)ₙ(C═O)O—, wherein N‡ represents a nitrogen from the polyalkylamine and when N‡ is a primary amine, R^{X} is hydrogen and when N‡ is a secondary amine, R^{X} is a covalent bond to the polyalkylamine. In certain embodiments, the linker is —N‡(R^{X})(C═O)—, —N‡(R^{X})(C═O)(CR₂)ₙ—, or —N‡(R^{X})(C═O)(CR₂)ₙ(C═O)—, or —N‡(R^{X})(C═O)(CR₂)ₙO—. In certain embodiments, R⁴ is selected from the group consisting of:

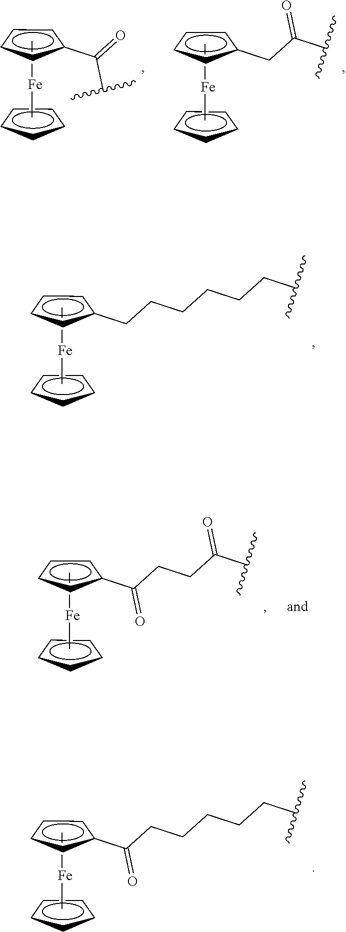

The polyalkylamine has the capacity to form one or more crosslinks with one or more third repeating units. The polyalkylamine can form intramolecular crosslinks with one or more third repeating units in the same enzyme-containing polymer chain; can form intermolecular crosslinks with one or more third repeating units in different enzyme-containing polymer chains; and combinations thereof. In instances in which the polyalkylamine forms a crosslink with a one more third repeating unit, the crosslinked structure can be represented as shown below:

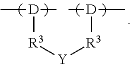

The polyalkylamine has the capacity to form one or more crosslinks, which can be represented as shown below:

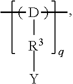

wherein q can be ≥1. In certain embodiments, q is 1-50; 1-40; 1-30; 1-20; 1-10; 1-5; or 1-3.

In certain embodiments, the first repeating unit has the Formula IIIa:

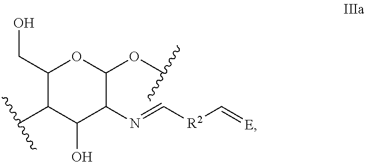

the second repeating unit has the Formula IIIb:

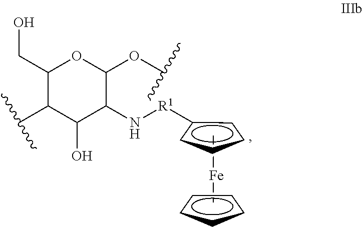

the third repeating unit has the Formula IIIc:

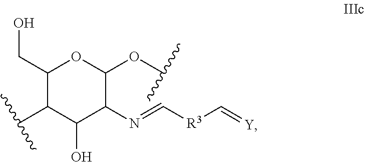

wherein

R¹ is —(C═O)—, —(C═O)N(R)—, —(CR₂)ₙ—, —(CR₂)ₙ(C═O)—, —(C═O)(CR₂)ₙ—, —(C═O)(CR₂)ₙ(C═O)—, —(CR₂)ₙO(CR₂)ₘ—, —(CR₂)ₙS(CR₂)ₘ—, —(CR₂)ₙO—, —(CR₂)ₙ(C═O)O—, —(C═O)(CR₂)ₙO—, —(C═O)(CR₂)ₙ(C═O)O—, —(CR₂)ₙO(CR₂)ₘO—, —(CR₂)ₙS(CR₂)ₘO—;

R² is —N*═CH(CR₂)ₙCH═N**—, —N*═CH(CR₂)ₙO(CR₂)ₘCH═N**—, or —N*═CH(CR₂)ₙS(CR₂)ₘCH═N**—;

R³ is —N*═CH(CR₂)ₙCH═N**—, —N*═CH(CR₂)ₙO(CR₂)ₘCH═N**—, or —N*═CH(CR₂)ₙS(CR₂)ₘCH═N**—;

R for each occurrence is independently hydrogen or lower alkyl;

m for each occurrence is independently a whole number selected between 1-20;

n for each occurrence is independently a whole number selected between 1-20; and

Y is a polyalkylamine comprising at least one metal complex, wherein the polyalkylamine optionally crosslinks at least two of the third repeating units.

In certain embodiments, the first repeating unit has the Formula IIIa, the second repeating unit has the Formula IIIb, the third repeating unit has the Formula IIIc, wherein R¹ is —(C═O)—, —(C═O)(CH₂)ₙ—, —(C═O)(CH₂)ₙ(C═O)—, —(CH₂)ₙ(C═O)O—, or —(C═O)(CH₂)ₙO—; R² is —(CH₂)ₙ—; R³ is —(CH₂)ₙ—; and Y is branched polyethylenimine comprising at least one primary or secondary amine covalently bonded to a moiety having the structure:

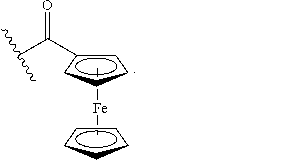

In certain embodiments, the first repeating unit has the Formula IIIa, the second repeating unit has the Formula IIIb, the third repeating unit has the Formula IIIc, R¹ is —(C═O)—; R² is —(CH₂)₃—; and R³ is —(CH₂)₃—.

In certain embodiments, the enzyme-containing polymer further comprises a fourth repeating unit of Formula IIId:

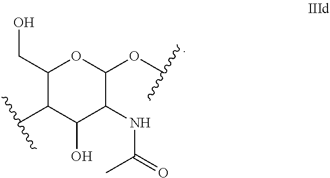

In certain embodiments, up to 40% of the repeating units in the enzyme-containing polymer are the fourth repeating unit. In certain embodiments, between 0.1% to 40%; 5% to 40%; 10% to 40%; or 20% to 40% of the repeating units in the enzyme-containing polymer are the fourth repeating unit.

In certain embodiments, the enzyme-containing polymer further comprises a fifth repeating unit of Formula IIIe:

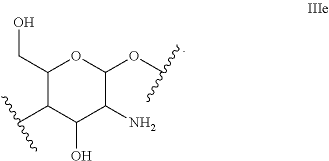

In certain embodiments, the ratio between the fourth repeating unit of Formula IIId and the fifth repeating unit of Formula IIIe is at least 1:3. The ratio of the first repeating unit, the second repeating unit and the third repeating unit will sum up to the total repeating units of Formula IIIe. In certain embodiments, the enzyme containing polymer may contain up to 5% of the fifth repeating unit of Formula IIIe that remain unreacted.

Also provided herein is a redox polymer. The redox polymer is a useful synthetic intermediate for preparing the enzyme-containing polymers described herein. In certain embodiments, the redox polymer comprises a first repeating of Formula IIa:

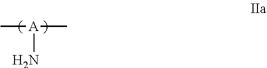

and a second repeating unit of Formula IIb:

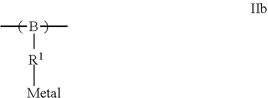

or conjugate salts thereof, wherein

each of A and B independently a 2-amino monosaccharide;

Metal is a metal complex having a redox potential lower than hydrogen peroxide under physiological conditions;

R¹ is —N*(R)(C═O)—, —N*(R)(C═O)N(R)—, —N*(R)(CR₂)ₙ—, —N*(R)(CR₂)ₙ(C═O)—, —N*(R)(C═O)(CR₂)ₙ—, —N*(R)(C═O)(CR₂)ₙ(C═O)—, —N*(R)(CR₂)ₙO(CR₂)ₘ—, —N*(R)(CR₂)ₙS(CR₂)ₘ—, —N*(R)(CR₂)ₙO—, —N*(R)(CR₂)ₙ(C═O)O—, —N*(R)(C═O)(CR₂)ₙO—, —N*(R)(C═O)(CR₂)ₙ(C═O)O—, —N*(R)(CR₂)ₙO(CR₂)ₘO—, —N*(R)(CR₂)ₙS(CR₂)ₘO—, —N*═CH(CR₂)ₙ—, —N*═CH(CR₂)ₙ(C═O)—, —N*═CH(CR₂)ₙO(CR₂)ₘ—, —N*═CH(CR₂)ₙS(CR₂)ₘ—, —N*═CH(CR₂)ₙO—, —N*═CH(CR₂)ₙ(C═O)O—, —N*═CH(CR₂)ₙO(CR₂)ₘO—, or —N*═CH(CR₂)ₙS(CR₂)ₘO—, wherein N* represents the nitrogen at the 2 position of the 2-amino monosaccharide;

R for each occurrence is independently hydrogen or lower alkyl;

m for each occurrence is independently a whole number selected between 1-20; and

n for each occurrence is independently a whole number selected between 1-20.

In certain embodiments of the redox polymer, the definitions of A, B, R¹, R, m, n, and Metal are the same as previously defined.

In certain embodiments of the redox polymer, the first repeating unit has the Formula IIIe:

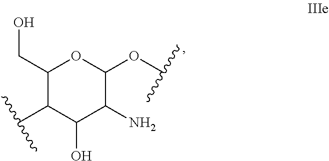

and the second repeating unit has the Formula IVb:

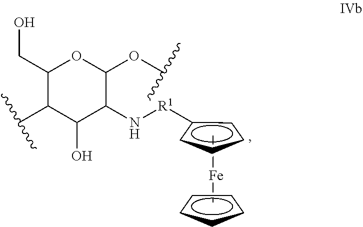

wherein R¹ is —(C═O)—, —(C═O)(CH₂)ₙ—, —(C═O)(CH₂)ₙ(C═O)—, —(CH₂)ₙ(C═O)O—, or —(C═O)(CH₂)ₙO—.

In certain embodiments of the redox polymer, the second repeating unit has Formula IVb and R¹ is —(C═O)—.

In certain embodiments, the redox polymer further comprises a third repeating unit of Formula IIId:

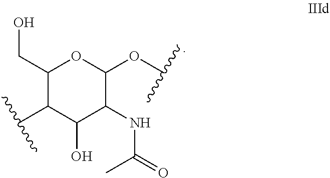

In certain embodiments, up to 40% of the repeating units in the redox polymer are the third repeating unit. In certain embodiments, between 0.1% to 40%; 5% to 40%; 10% to 40%; or 20% to 40% of the repeating units in the redox polymer are the third repeating unit.

The redox polymer may comprise side chains with functional groups that facilitate cross-linking with other molecules with suitable functional groups. This allows the polymer to be attached to a wide variety of molecules as well. Combining these two characteristics, these polymers are well adapted for use in applications requiring electron mediation, such as enzyme electrodes used in biosensors and biofuel cells, as well as enzymatic synthesis carried out in electroenzyme reactors.

The redox polymer may be a polymeric redox mediator which may be considered as an electrochemical activator. The electrochemical activator may be represented as monomeric electrochemical activator. The electrochemical activator may contain redox-active metal ion. The redox metal ion may be selected from the group consisting of iron, silver, gold, copper, nickel, cobalt, osmium or ruthenium ions and mixtures thereof. The electrochemical activator may preferably be a ferrocenyl derivative. The ferrocenyl derivative may be water soluble.

The redox polymer may be incorporated accordingly into the membrane. The redox polymer may have a chemical structure which prevents or substantially reduces the diffusional loss of the redox species during the period of time that the sample is being analysed or when the sensor is being clipped on a tissue. The diffusional loss of the redox mediator may be reduced by rendering the redox polymer non-releasable from the working electrode in the sensor. The one type of non-releasable polymeric redox mediator may comprise of a redox species covalently attached to a polymeric compound. The redox polymer may be a transition metal compound having a redox-active transition metal based pendant group covalently bound to a suitable polymer backbone. The polymer backbone may or may not be electroactive. The polymer backbone may be an amino-containing polysaccharide group.

Advantageously, the redox polymer may be able to mediate the electrical current flow between different electrodes such that the electrolysis (electrooxidation or electroreduction) of the analyte is smooth. More advantageously, the redox mediator may also enable the electrochemical analysis of the molecules of the analyte that are not suitable for direct electrochemical reaction on the working electrodes.

The redox polymer may have a molecular weight of between about 50,000 to about 190,000 Daltons, about 60,000 to about 190,000, about 70,000 to about 190,000, about 80,000 to about 190,000, about 90,000 to about 190,000, about 100,000 to about 190,000, about 110,000 to about 190,000, about 120,000 to about 190,000, about 130,000 to about 190,000, about 140,000 to about 190,000, about 150,000 to about 190,000, about 160,000 to about 190,000, about 170,000 to about 190,000, about 180,000 to about 190,000, about 50,000 to about 60,000, about 50,000 to about 70,000, about 50,000 to about 80,000, about 50,000 to about 90,000, about 50,000 to about 100,000, about 50,000 to about 110,000, about 50,000 to about 120,000, about 50,000 to about 130,000, about 50,000 to about 140,000, about 50,000 to about 150,000, about 50,000 to about 160,000, about 50,000 to about 170,000, about 50,000 to about 180,000 Daltons.

The redox polymer may have a ferrocene loading in the range of about 2 wt % to about 40 wt %, about 2 wt % to about 35 wt %, about 2 wt % to about 30 wt %, about 2 wt % to about 25 wt %, about 2 wt % to about 20 wt %, about 2 wt % to about 17 wt %, about 17 wt % to about 40 wt %, about 20 wt % to about 40 wt %, about 25 wt % to about 40 wt %, about 30 wt % to about 40 wt %, about 35 wt % to about 40 wt %, about 2 wt % to about 3 wt %, about 3 wt % to about 14 wt %, about 14 wt % to about 20 wt % or about 14 wt % to about 40 wt %.

The redox polymer may preferably have a high level of ferrocene loading, at least above wt 2%. A low level of ferrocene loading may usually impose a limit to the glucose concentration that can be measured.

The glucose concentration may be much higher than the mediating capacity of the ferrocene molecules present. Hence, the amperometric response that is generated may be limited by the small number of mediating ferrocene molecules, resulting in an inaccurate measurement. Therefore, by employing the redox polymer of formula (II) having a higher level of ferrocene loading, the upper limit of glucose concentrations that can be tested with the sensor is raised and thus smaller volume of sample is required.

Exemplary, non-limiting embodiments of a method of preparing an enzyme-containing polymer will now be disclosed.

The method may comprise the steps of:

(i) contacting a redox polymer as defined above and a polyalkylamine comprising at least one metal complex in the presence of a first cross-linker thereby forming a precursor of said enzyme-containing polymer, and (ii) contacting the precursor of said enzyme-containing polymer with an enzyme in the presence of a second cross-linker thereby forming said enzyme-containing polymer.

The contacting step in (i) may refer to a solution of redox polymer mixed with a solution of polyalkylamine comprising at least one metal complex in the presence of a first cross-linker. The solution of redox polymer may be prepared by dissolving the redox polymer in a phosphate-buffered saline (PBS) solution. The solution of polyalkylamine comprising at least one metal complex may be prepared by dissolving the polyalkylamine comprising at least one metal complex in an acidic solution, e.g., an acetic acid solution. The first cross-linker may be a reactant containing at least two aldehyde functional groups. The reactant containing at least two aldehyde functional groups may be a lower alkyl reactant containing an aldehyde functional group at each of two terminating ends of the lower alkyl reactant. The first cross-linker may be ethylene glycol diglycidyl ether, poly(ethylene glycol) diglycidyl ether or glutaraldehyde. In certain embodiments, the first cross-linker is glutaraldehyde. The first cross-linker solution may be prepared by dissolving the cross-linker in a phosphate-buffered saline (PBS) solution. The cross-linker solution may be further diluted in an aqueous solution before adding to the reaction mixture. The reaction time for step (i) may be in the range of about 1 to about 6 hours, about 2 to about 6 hours, about 3 to about 6 hours, about 4 to about 6 hours, about 5 to about 6 hours, about 1 to about 2 hours, about 1 to about 3 hours, about 1 to about 4 hours, about 1 to about 5 hours. The reaction time may preferably be 2 hours. The reaction may be performed at room temperature. The room temperature may be in the range of about 20° C. to about 25° C. or at a temperature of 20° C., 21° C., 22° C., 23° C., 24° C. or 25° C.

The contacting step in (ii) may refer to a solution of precursor of said enzyme-containing polymer obtained in step (i) mixed with a solution of the enzyme in the presence of a second cross-linker thereby forming the enzyme-containing polymer. The enzyme may be glucose oxidase or lactate oxidase. The solution of the enzyme may be prepared by mixing the enzyme with a PBS solution. The second cross-linker may be a reactant containing at least two aldehyde functional groups. The reactant containing at least two aldehyde functional groups may be a lower alkyl reactant containing an aldehyde functional group at each of two terminating ends of the lower alkyl reactant. The second cross-linker may be ethylene glycol diglycidyl ether, poly(ethylene glycol) diglycidyl ether or glutaraldehyde. In certain embodiments, the second cross-linker is glutaraldehyde. The reaction time for the method of preparing the enzyme-containing polymer may be in the range of about 20 minutes to about 20 hours, about 20 minutes to 60 minutes, about 1 to about 10 hours, about 1 to about 3 hours, about 1 to about 6 hours, about 3 to about 10 hours, about 6 to about 10 hours, about 10 to about 16 hours, about 11 to about 16 hours, about 12 to about 16 hours, about 13 to about 16 hours, about 14 to about 16 hours, about 15 to about 16 hours, about 10 to about 11 hours, about 10 to about 12 hours, about 10 to about 13 hours, about 10 to about 14 hours, about 10 to about 15 hours, or about 16 to about 20 hours. The reaction time may be 12 hours. The reaction may be performed at room temperature. The room temperature may be in the range of about 20° C. to about 25° C. or at a temperature of 20° C., 21° C., 22° C., 23° C., 24° C. or 25° C. The enzyme-containing polymer obtained in step (ii) may be applied and dried as a thin layer of film on a sensor chip.

In certain embodiments, the first and second cross-linker may be a natural cross-linker for proteins, collagen, gelatin, and chitosan. The first and second cross-linkers may be the same or different cross-linkers.

The method for preparing the redox polymer may further comprise the step of contacting a polysaccharide comprising a first repeating of Formula IIa

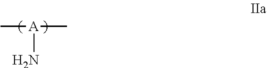

or conjugate salt thereof, wherein

A is a 2-amino monosaccharide;

with a ferrocenyl derivative in the presence of one or more reagents and isolating the redox polymer that is formed for the next step.

The polysaccharide of the redox polymer may be from chitosan solids or chitosan flakes. The one or more reagents for the method of preparing the redox polymer may be any reagent used for an amide formation between a carboxylic acid and an amine. The reagent may be a coupling reagent used to form amide bonds. The coupling reagent may be selected from the group consisting N-ethyl-N′-(3-dimethylaminopropyl)carbodiimide hydrochloride, dicyclohexylcarbodiimide, diisopropylcarbodiimide, benzotriazol-1-yloxy-tris (dimethylamino)-phosphonium hexafluorophosphate, benzotriazol-1-yloxy-tripyrrolidino-phosphonium hexafluorophosphate and (2-(7-aza-1H-benzotriazol-1-yl)-N,N,N′,N′-tetramethylaminium tetrafluoroborate/hexafluorophosphate. The coupling reagent may preferably be N-ethyl-N′-(3-dimethylaminopropyl)carbodiimide hydrochloride. The one or more reagents for the method of preparing the redox polymer may comprise of an additive. The additive may be a reagent that will facilitate the amide formation between the carboxylic acid and the amine. The additive may be selected from the group consisting of N-hydroxysuccinimide, 1-hydroxybenzotriazole, hydroxy-3,4-dihydro-4-oxo-1,2,3-benzotriazine, 1-hydroxy-7-aza-1H-benzotriazole, 1-hydroxybenzotriazole-6-sulfonamidomethyl resin. HCl and 4-(N,N-dimethylamino)pyridine. In certain embodiments, the additive is N-hydroxysuccinimide. The selection of the coupling reagent and additive is within the skill of a person of ordinary skill in the art. When the polysaccharide is contacting with the ferrocenyl derivative, the polysaccharide may be dissolved in an aqueous solution and the ferrocenyl derivative may be dissolved in a solvent. The aqueous solution may be deionized water. The solvent may be an organic solvent. The organic solvent may be methanol, ethanol, propanol, ethyl acetate, dichloromethane or methylene chloride, chloroform, tetrahydrofuran, acetone, acetonitrile, N,N,-dimethylformamide, dimethyl sulfoxide or 1,4-dioxane. In certain embodiments, the solvent is methanol or anhydrous methanol. The reaction time for the method of preparing the redox polymer may be in the range of about 1 to about 20 hours, about 1 to about 3 hours, about 1 to about 6 hours, about 3 to about 10 hours, about 6 to about 10 hours, about 1 to about 10 hours, about 10 to about 16 hours, about 11 to about 16 hours, about 12 to about 16 hours, about 13 to about 16 hours, about 14 to about 16 hours, about 15 to about 16 hours, about 10 to about 11 hours, about 10 to about 12 hours, about 10 to about 13 hours, about 10 to about 14 hours, about 10 to about 15 hours, or about 16 to about 20 hours. The reaction time may be 12 hours. The reaction may be performed under inert atmosphere at room temperature, preferably under nitrogen or argon atmosphere. The room temperature may be in the range of about 20° C. to about 25° C. or at a temperature of 20° C., 21° C., 22° C., 23° C., 24° C. or 25° C. The method of isolating the redox polymer may be the standard isolating techniques that are within the skill of a person of ordinary skill in the art. The base that was used in the isolation process may be any inorganic base, preferably sodium hydroxide. The isolation process may require other aqueous solution or solvent as necessary, preferably anhydrous methanol. The redox polymer may be further dissolved in an acidic solution to be used in the next step. The acidic solution may be prepared from an organic acid, preferably acetic acid.

The method for preparing the polyalkylamine comprising at least one metal complex may further comprise contacting a ferrocenyl derivative with a polyalkylamine under basic reaction conditions and isolating the polyalkylamine comprising at least one metal complex that is formed for the next step.

The polyalkylamine may be selected as mentioned above and may be dissolved in an organic solvent, preferably anhydrous methanol. The ferrocenyl derivative may be selected as mentioned above and may be dissolved in an organic solvent, preferably anhydrous methanol. The step of contacting the ferrocenyl derivative with the polyalkylamine may be in the form of adding a solution of ferrocenyl derivative dropwise into a solution of polyalkylamine under basic conditions. The basic conditions may comprise of an organic base in the solvent, preferably triethylamine in anhydrous methanol. The reaction time for the method of preparing the polyalkylamine comprising at least one metal complex may be in the range of about 1 to about 20 hours, about 1 to about 3 hours, about 1 to about 6 hours, about 3 to about 10 hours, about 6 to about 10 hours, about 1 to about 10 hours, about 10 to about 16 hours, about 11 to about 16 hours, about 12 to about 16 hours, about 13 to about 16 hours, about 14 to about 16 hours, about 15 to about 16 hours, about 10 to about 11 hours, about 10 to about 12 hours, about 10 to about 13 hours, about 10 to about 14 hours, about 10 to about 15 hours, or about 16 to about 20 hours. The reaction time may be 12 hours.

The reaction may be performed under inert atmosphere at room temperature, preferably under nitrogen or argon atmosphere. The room temperature may be in the range of about 20° C. to about 25° C. or at a temperature of 20° C., 21° C., 22° C., 23° C., 24° C. or 25° C. The method of isolating the polyalkylamine comprising at least one metal complex may be the standard isolating techniques that are within the skill of a person of ordinary skill in the art. The solvents that are used in the isolation process may be any organic solvents, such as those selected from the group consisting of chloroform, hexane, ethyl acetate, dichloromethane or methylene chloride and acetonitrile. In certain embodiments, the organic solvent used for the isolation process is a combination of chloroform and hexane. The chloroform/hexane mixed solvents may be in a volume/volume (v/v) ratio, in the range of 1:5 v/v, 1:4 v/v, 1:3 v/v, 1:2 v/v or 1:1 v/v, more preferably in 1:4 v/v ratio. The isolation process may require other aqueous solution or solvent as necessary, preferably methanol. The polyalkylamine comprising at least one metal complex may be further dissolved in a buffer solution to be used in the next step. The buffer solution may be prepared as phosphate-buffered saline (PBS) solution.

In certain embodiments, the method may comprise contacting the redox polymer as defined above with an enzyme in the presence of a cross-linker thereby forming the enzyme-containing polymer.

The method may further comprise the steps of: a) preparing a polysaccharide solution by mixing a polysaccharide precursor with an acid under stirring for a period of time and at a specific temperature; b) dissolving ferrocenyl derivative in a mixture solution of solvent and alcohol; c) mixing the polysaccharide solution and the ferrocenyl solution, followed by heating for a period of time, and isolating the redox polymer that is formed; and d) mixing a solution of the redox polymer formed in step (c) with an enzyme in the presence of a cross-linker to thereby form said enzyme-containing polymer.

The polysaccharide precursor may be chitosan solids or chitosan flakes. The acid may be an inorganic acid or an organic acid. The acid may be acetic acid, formic acid, propionic acid, butyric acid, valeric acid, caproic acid and benzoic acid. In certain embodiments, the acid is acetic acid.

The reaction time in step (a) may vary between 30 minutes to 12 hours. It may vary in a range of about 30 minutes to about 6 hours, about 1 hour to about 6 hours, about 1.5 hours to about 6 hours, about 2 hours to about 6 hours, about 2.5 hours to about 6 hours, about 3 hours to about 6 hours, about 3.5 hours to about 6 hours, about 4 hours to about 6 hours, about 5 hours to about 6 hours, about 30 minutes to about 5 hours, about 30 minutes to about 4 hours, about 30 minutes to about 3 hours, about 30 minutes to about 2 hours, about 30 minutes to about 1 hour, about 6 hours to about 12 hours, about 7 hours to about 12 hours, about 8 hours to about 12 hours, about 9 hours to about 12 hours, about 10 hours to about 12 hours, about 11 hours to about 12 hours, about 6 hours to about 7 hours, about 6 hours to about 8 hours, about 6 hours to about 9 hours, about 6 hours to about 10 hours, about 6 hours to about 11 hours or preferably may be about 30 minutes, about 1 hour, about 2 hours, about 3 hours, about 4 hours, about 5 hours, or about 6 hours or more preferably about 3 hours.

The first reaction temperature in step (a) may be in the range of about 20° C. to about 25° C. or at a temperature of 20° C., 21° C., 22° C., 23° C., 24° C. or 25° C. or preferably at room temperature.

In step (b), the reaction may be carried out in a solvent. The solvent may be selected from the group consisting of ethyl acetate, dichloromethane or methylene chloride, tetrahydrofuran (THF), acetone, acetonitrile, N,N,-dimethylformamide, dimethyl sulfoxide, 1,4-dioxane, and combinations thereof. The solvent may preferably be ethyl acetate or acetone. The alcohol may be a linear alkyl-alcohol or branched alkyl-alcohol selected from the group consisting of methanol, ethanol, propanol, butanol, pentanol, 3-pentanol or hexanol. In certain embodiments, the alcohol is methanol.

The reaction mixture condition in step (c) may be acidic, basic or neutral condition. The reaction mixture condition may preferably be under basic pH condition. When under the basic condition, the base that generates the basic pH condition in step (c) may be an inorganic or an organic base. The inorganic base may be an inorganic compound. The inorganic base may be selected from the group consisting of potassium carbonate, potassium phosphate tribasic or cesium carbonate. The inorganic base may preferably be potassium carbonate. The organic base may be an organic compound. The organic base may be a proton acceptor containing organic base. The organic base may be selected from the group consisting of triethylamine, Hunig's base, pyridine, methyl amine, imidazole, benzimidazole and histidine. The organic base may preferably be triethylamine. The reaction mixture condition may preferably be under neutral condition. When under the neutral condition, a reducing agent may be added to the solution of polysaccharide and ferrocenyl in step (c). The reducing agent may be an element or compound that loses an electron to another chemical species in a redox chemical reaction. When the reducing agent is losing electrons, the reducing agent may be said to have been oxidized. The reducing agent may be selected from the group consisting of lithium aluminium hydride, sodium borohydride, iron(II) sulphate, diisobutylaluminium hydride and tin(II) chloride.

The heating in step (c) may be carried out at an elevated reaction temperature that is suitable with the boiling point of the solvent. The elevated reaction temperature may be in a range of about 50° C. to about 80° C., about 50° C. to about 55° C., about 50° C. to about 60° C., about 50° C. to about 65° C., about 50° C. to about 70° C., about 50° C. to about 75° C., about 55° C. to about 80° C., about 60° C. to about 80° C., about 65° C. to about 80° C., about 70° C. to about 80° C. or about 75° C. to about 80° C. The elevated reaction temperature may be considered as heated to a refluxing temperature of the solvent.

The reaction time in step (c) may vary in a range of about 20 hours to about 28 hours, about 20 hours to about 27 hours, about 20 hours to about 26 hours, about 20 hours to about 25 hours, about 20 hours to about 24 hours, about 20 hours to about 23 hours, about 20 hours to about 22 hours, about 20 hours to about 21 hours, about 21 hours to about 28 hours, about 22 hours to about 28 hours, about 23 hours to about 28 hours, about 24 hours to about 28 hours or about 25 hours to about 28 hours, about 26 hours to about 28 hours, about 27 hours to about 28 hours or preferably about 24 hours.

After the reaction is complete, the reaction solution containing the redox polymer may be extracted with a nonpolar solvent. The nonpolar solvent may be selected from the group consisting of diethyl ether, hexane, pentane, cyclohexane, toluene and chloroform. The nonpolar solvent may preferably be diethyl ether.

The redox polymer may then be isolated by drying the reaction solution over a drying agent, filtered and concentrated under pressure. The drying step which is also considered to be an extraction step may be carried out at least once twice, three times, four times or up to five times. The drying agent may be selected from the group consisting of anhydrous sodium sulfate, magnesium sulfate, calcium chloride or calcium sulphate. The combined reaction solution may be concentrated under reduced pressure. The reaction residue (reaction crude product) may be purified by washing and further drying.

In step (d), the redox polymer formed in step (c) is dissolved in an aqueous solution and reacted with an enzyme in the presence of the cross-linker to form the enzyme-containing polymer. The aqueous solution may be a buffer solution. The buffer solution may be an aqueous solution consisting of a mixture of a weak acid and its conjugate base or vice versa. The pH value changes very little when a small amount of strong acid or base is added to the solution. The buffer solution may be used as a means of monitoring the pH value at a nearly constant value. The buffer solution may be selected from the group consisting citric acid-Na₂HPO₄ solution, citric acid-sodium citrate solution and sodium acetate-acetic acid solution. The buffer solution may preferably be sodium acetate-acetic (HOAc/OAc) solution.

The compounds as defined above may be made according to the general processes as disclosed above or according to the general principles of the working examples. The compounds as defined above may be prepared by other alternative chemistry reaction and not limited to the general processes as disclosed above.

The enzyme-containing polymer may then be used to form a sensing layer on a sensor that can be used to monitor for failure of a tissue. The enzyme-containing polymer may be cured on a sensor surface within a time period selected from the range consisting of about 10 to about 14 hours, about 11 to about 14 hours, about 12 to about 14 hours, about 13 to about 14 hours, about 10 to about 11 hours, about 10 to about 12 hours, about 10 to about 13 hours or preferably about 12 hours.

Exemplary, non-limiting embodiments of a system to monitor the physiological changes of surface and buried tissue in order to detect early signs of failure will now be disclosed. The monitoring system may comprise of a sensor as defined herein; and a monitor as defined here, wherein the receiver of the monitor is arranged in use to receive an output of the sensor. Here, the tissue may refer to a flap or an organ, such as a transplanted organ.

The system may comprise one or more electrochemical metabolite biosensors connected to a sensor reader with an associated algorithm, which directly measures metabolite concentration within a tissue. The system seeks to provide high sensitivity and accuracy in early detection of a failing tissue with good long term stability for at least 5 to 7 days. The system is intended to be used in the wards and Intensive Care Units (ICUs) for monitoring tissue(s) of patients, for example who just had flap reconstruction surgery. The advantages of the system include accuracy, ease of use (i.e. very little training required), automated (i.e. no manual supervision required), fast response time (within 15 minutes from vascular thrombosis event) and continuous monitoring (5 to 7 days). During data recording, the electrodes are fixed.

In an implementation, a tissue sensor is placed at or within a tissue. It is interpreted that the tissue is starting to fail if the amount of a second metabolite (such as lactate) from the tissue as read by the tissue sensor rises above a certain threshold as compared to that from the control value and if the amount of a first metabolite (such as glucose) from the tissue as read by the tissue sensor drops below a certain threshold as compared to that from the control value. This control value may be obtained by means of a control sensor that is placed on or within a normal, healthy part of the same patient such that any deviation between the testing sensor and the control sensor can be taken as indicative of the health or failure of the tissue. It is envisaged that other possible control readings may be used or taken, to determine, analyse and monitor the tissue failure condition based on varying metabolite compositions and/or chemical component/compound levels and/or other characteristics. Therefore, other alternative algorithms can be used to determine the tissue failure condition.

According to one embodiment, an electrochemical metabolite biosensor to detect vascular thrombosis in a tissue through continuous monitoring of the level change of blood metabolites such as glucose and lactate is provided. A biosensor may be effective in clinical diagnostics as it can transform the information from a biological event to a measurable signal. A biosensor may be composed of a biological recognition element which must be selective, a transducer to generate the measurable signal and a signal processing unit. Electrochemical biosensors may contain a biological recognition element on the electrode/transducer which reacts with an analyte and then produces a corresponding electrochemical signal. The advantages of electrochemical biosensors are that they are inexpensive, provide a fast response, have high sensitivity with a simple construction. Regarding blood metabolites, glucose and lactate, enzyme based amperometric biosensors combine electrochemical technology with specificity of enzyme. Taking glucose as an example, the sensing principle employed is based on the reaction of glucose oxidase (GOx) catalyzing the oxidization of glucose to gluconolactone. In vivo continuous glucose monitoring may be implemented via an implantable glucose sensor based on gold or carbon materials for subcutaneous monitoring. Such a device may be inserted into the skin and display real time glucose concentration. A disposable sensor yields a reading every minute and often can be used for three to seven days. The system tracks glucose level in interstitial fluid of subcutaneous tissue instead of measuring blood glucose directly. Herein, a real-time electrochemical biosensor for continuous monitoring of interstitial fluid metabolites (glucose and lactate for this embodiment) is provided.

One or more sensors may be implanted into the surface of the reconstructed tissue (called tissue sensor) by surgeons at the end of a tissue reconstruction surgery. A control sensor may be implanted in an area of healthy non-flap tissue. The difference between the readings from both sensors determines if the tissue is healthy or failing. When a tissue is failing, the readings from both sensors, tissue and control sensors may follow a pattern. For instance, when the metabolites are glucose and lactate, the difference or deviation is at least 10% decrease in glucose level and/or at least 10% increase in lactate level. The difference or deviation between the tissue sensor and the control sensor of the at least two metabolites may follow the same direction (that is the at least two metabolites may all increase, or the at least two metabolites may all decrease). Alternatively, the direction that each metabolite takes may be opposite to each other. The difference or deviation between the tissue sensor and the control sensor may depend on the type of metabolite being detected as well as the type of tissue being monitored. An electrochemical glucose and lactate sensor in contact with the tissue may detect metabolite changes within minutes of vessel blockage.

The method for monitoring failure of a tissue on a patient may comprise of the steps:
(i) providing a first sensor as defined herein on or within said tissue, said first sensor being capable of detecting and measuring the amount of a first metabolite;
(ii) providing a second sensor as defined herein on a control region of said patient, said control region being separate from said tissue and wherein said second sensor is capable of detecting and measuring the amount of said first metabolite;
(iii) providing a third sensor as defined herein on or within said tissue, said third sensor being capable of detecting and measuring the amount of a second metabolite and wherein said third sensor is the same as or different from the first sensor;
(iv) providing a fourth sensor as defined herein on said control region of said patient, said fourth sensor being capable of detecting and measuring the amount of said second metabolite and wherein said fourth sensor is the same as or different from the second sensor;
(v) monitoring the amounts of said first metabolite measured by both said first and second sensors for a period of time;
(vi) monitoring the amounts of said second metabolite measured by both said third and fourth sensors for a period of time;

wherein an at least 10% decrease in the amount of said first metabolite as measured by said first sensor as compared to the amount of said first metabolite as measured by said second sensor; and at least 10% increase in the amount of said second metabolite as measured by said third sensor as compared to the amount of said second metabolite as measured by said fourth sensor, is indicative that said tissue is prone to failure.

The method for monitoring failure of a tissue on a patient may comprise of the steps:
(i) providing a first sensor on or within said tissue, said first sensor being capable of detecting and measuring the amount of a first metabolite;
(ii) providing a second sensor on a control region of said patient, said control region being separate from said tissue and wherein said second sensor is capable of detecting and measuring the amount of said first metabolite;
(iii) providing a third sensor on or within said tissue, said third sensor being capable of detecting and measuring the amount of a second metabolite and wherein said third sensor is the same as or different from the first sensor;
(iv) providing a fourth sensor on said control region of said patient, said fourth sensor being capable of detecting and measuring the amount of said second metabolite and wherein said fourth sensor is the same as or different from the second sensor;
(v) monitoring the amounts of said first metabolite measured by both said first and second sensors for a period of time;
(vi) monitoring the amounts of said second metabolite measured by both said third and fourth sensors for a period of time;

wherein the amount of said first metabolite as measured by said first sensor and the amount of said first metabolite as measured by said second sensor are substantially the same; and at least 10% increase in the amount of said second metabolite as measured by said third sensor as compared to the amount of said second metabolite as measured by said fourth sensor, is indicative that said tissue is prone to failure.

The amount of said first metabolite as measured by said first sensor and the amount of said first metabolite as measured by said second sensor may be substantially the same while said second metabolite as measured by said third sensor and said fourth sensor increases.

The method for monitoring failure of a tissue on a patient may comprise of the steps:
(i) providing a first sensor on or within said tissue, said first sensor being capable of detecting and measuring the amount of a first metabolite;
(ii) providing a second sensor on a control region of said patient, said control region being separate from said tissue and wherein said second sensor is capable of detecting and measuring the amount of said first metabolite;
(iii) providing a third sensor on or within said tissue, said third sensor being capable of detecting and measuring the amount of a second metabolite and wherein said third sensor is the same as or different from the first sensor;
(iv) providing a fourth sensor on said control region of said patient, said fourth sensor being capable of detecting and measuring the amount of said second metabolite and wherein said fourth sensor is the same as or different from the second sensor;
(v) monitoring the amounts of said first metabolite measured by both said first and second sensors for a period of time;
(vi) monitoring the amounts of said second metabolite measured by both said third and fourth sensors for a period of time;

wherein an at least 10% decrease in the amount of said first metabolite as measured by said first sensor as compared to the amount of said first metabolite as measured by said second sensor; and the amount of said second metabolite as measured by said third sensor and the amount of said second metabolite as measured by said fourth sensor are substantially the same, is indicative that said tissue is prone to failure.

The amount of said second metabolite as measured by said third sensor and the amount of said second metabolite as measured by said fourth sensor may be substantially the same while said first metabolite as measured by said first sensor and said second sensor decreases.

The method for monitoring failure of a tissue on a patient may comprise of the steps of:
(i) providing a first sensor on or within said tissue, said first sensor being capable of detecting and measuring the amount of a first metabolite;
(ii) providing a second sensor on a control region of said patient, said control region being separate from said tissue and wherein said second sensor is capable of detecting and measuring the amount of said first metabolite;
(iii) providing a third sensor on or within said tissue, said third sensor being capable of detecting and measuring the amount of a second metabolite and wherein said third sensor is the same as or different from the first sensor;
(iv) providing a fourth sensor on said control region of said patient, said fourth sensor being capable of detecting and measuring the amount of said second metabolite and wherein said fourth sensor is the same as or different from the second sensor;
(v) monitoring the amounts of said first metabolite measured by both said first and second sensors for a period of time;
(vi) monitoring the amounts of said second metabolite measured by both said third and fourth sensors for a period of time;

wherein an at least 10% decrease in the amount of said first metabolite as measured by said first sensor as compared to the amount of said first metabolite as measured by said second sensor; and an at least 10% decrease in the amount of said second metabolite as measured by said third sensor as compared to the amount of said second metabolite as measured by said fourth sensor, is indicative that said tissue is prone to failure.

The method for monitoring failure of a tissue on a patient may comprise of the steps of:
(i) providing a first sensor on or within said tissue, said first sensor being capable of detecting and measuring the amount of a first metabolite;
(ii) providing a second sensor on a control region of said patient, said control region being separate from said tissue and wherein said second sensor is capable of detecting and measuring the amount of said first metabolite;
(iii) providing a third sensor on or within said tissue, said third sensor being capable of detecting and measuring the amount of a second metabolite and wherein said third sensor is the same as or different from the first sensor;
(iv) providing a fourth sensor on said control region of said patient, said fourth sensor being capable of detecting and measuring the amount of said second metabolite and wherein said fourth sensor is the same as or different from the second sensor;
(v) monitoring the amounts of said first metabolite measured by both said first and second sensors for a period of time;
(vi) monitoring the amounts of said second metabolite measured by both said third and fourth sensors for a period of time;

wherein an at least 10% increase in the amount of said first metabolite as measured by said first sensor as compared to the amount of said first metabolite as measured by said second sensor; and an at least 10% increase in the amount of said second metabolite as measured by said third sensor as compared to the amount of said second metabolite as measured by said fourth sensor, is indicative that said tissue is prone to failure.

The amount or level of the first metabolite may be decreased by at least 10%, at least 15%, at least 20%, at least 25%, at least 30%, at least 35%, at least 40%, at least 45%, or at least 50%. The first metabolite may be glucose.

The amount or level of the second metabolite may be increased by at least 10%, at least 11%, at least 12%, at least 13%, at least 14%, at least 15%, at least 16%, at least 17%, at least 18%, at least 19%, at least 20%, at least 25%, at least 30%, at least 35%, at least 40%, at least 45% or at least 50%. The second metabolite may be lactate.

By having a control sensor (as represented by the second and fourth sensors) that is able to measure the baseline or the natural amount or resting amounts of the first metabolite and second metabolite, and the use of the percentage deviation from these baseline amounts (as measured by the first and third sensors) to determine whether a tissue (which can be a flap or a transplanted organ) is prone to failure or potentially may fail, this may be considered as a self-referencing or self-calibrating method to determine whether a tissue is prone to failure. Thus, by having a control sensor that forms a baseline in the measurement of a first metabolite and second metabolite, and obtaining a deviation from the baseline measurement, this may allow for a more accurate system with higher accuracy. By determining the values of both first metabolite and second metabolite and monitoring those values, this enables a more accurate determination to be made.

In an implementation, tissue failure can be determined by an algorithm that takes into account (i) deviation of the tissue value compared to the control sensor value, as well as (ii) a historical trend of the tissue sensor deviations from the control sensor over a predetermined period e.g. 2 minutes, 5 minutes, 10 or 15 minutes, to minimize effects of a noisy signal.

For example, during a particular time window (e.g. x to x+5 seconds), deviation of the tissue sensor value compared to the control sensor value is 30%. For the following 5 seconds (i.e. x+5 to x+10 seconds), deviation of the tissue sensor value compared to the control sensor value is 10%. However, deviation of the tissue sensor value was in the range of 5% to 15% during the previous 10 minutes (i.e. x−10 minutes). Based on the historical trends, the algorithm may determine that there was noise in the signal during x to x+5 seconds, and the deviation of 30% during that time window may be normalized based on the deviation range during x−10 minutes.

Exemplary, non-limiting embodiments of a monitor will now be disclosed.

The monitor may comprise of a receiver module configured to receive a sensor output of a sensor and a control output of another sensor; a processor module configured to receive a first metabolite concentration value corresponding to the sensor output and a first control value corresponding to the control output from the receiver module, wherein the processor module is configured to: compare the first metabolite concentration value against the first control value; and generate a first alarm signal on a condition that a difference between the first metabolite concentration value and the first control value is above a first pre-determined value.

The processor module of the monitor may further be configured to receive a second metabolite concentration value corresponding to the sensor output and a second control value corresponding to the control output from the receiver module, and wherein the processor module is further configured to: compare the second metabolite concentration value against the second control value; and generate a second alarm signal on a condition that a difference between the second metabolite concentration value and the second control value is above a second pre-determined value.

The processor module of the monitor may be configured to generate a first alarm signal on the condition that the difference between the first metabolite concentration value and the first control value may be at least 10% different from the first pre-determined value.

The processor module of the monitor may be configured to generate a second alarm signal on the condition that the difference between the second metabolite concentration value and the second control value may be at least 10% different from the second pre-determined value.

The method for monitoring failure of a tissue on a patient may comprise of the steps:
(i) providing a first sensor on or within said tissue, said first sensor being capable of detecting and measuring the amount of a first metabolite;
(ii) providing a second sensor on a control region of said patient, said control region being separate from said tissue and wherein said second sensor is capable of detecting and measuring the amount of said first metabolite;
(iii) providing a third sensor on or within said tissue, said third sensor being capable of detecting and measuring the amount of a second metabolite and wherein said third sensor is the same as or different from the first sensor;
(iv) providing a fourth sensor on said control region of said patient, said fourth sensor being capable of detecting and measuring the amount of said second metabolite and wherein said fourth sensor is the same as or different from the second sensor;
(v) monitoring the amounts of said first metabolite measured by both said first and second sensors for a period of time;
(vi) monitoring the amounts of said second metabolite measured by both said third and fourth sensors for a period of time;

wherein an at least 10% decrease in the amount of said first metabolite as measured by said first sensor as compared to the amount of said first metabolite as measured by said second sensor; and at least 10% increase in the amount of said second metabolite as measured by said third sensor as compared to the amount of said second metabolite as measured by said fourth sensor, is indicative that said tissue is prone to failure.

Exemplary, non-limiting embodiments of a sensor and a method of manufacturing the sensor will now be disclosed.

In one embodiment, the sensor may include a substrate, a first sensor electrode on the substrate, a first sensing layer on the first sensor electrode, and a reference electrode on the substrate. The first sensing layer includes a first enzyme-containing polymer according to embodiments disclosed herein.

The sensor may further include a second sensor electrode on the substrate and a second sensing layer on the second sensor electrode. The second sensing layer includes a second enzyme-containing polymer according to embodiments disclosed herein.

The first and/or second enzyme-containing polymer may have a thickness of in the range of about 0.010 mm to about 0.300 mm. The first and/or second sensor electrode may be an electrode having an inert material that allows conductivity. The first and/or second sensor electrode may comprise gold, and the reference electrode may comprise silver-silver chloride (Ag/AgCl). The reference electrode functions as a reference by providing a stable reference potential.

In an implementation having both (i) a first sensor electrode and associated first sensing layer, and (ii) second sensor electrode and associated second sensing layer, the (i) first sensor electrode and associated first sensing layer detects the concentration of a first metabolite while the (ii) second sensor electrode and associated second sensing layer detects the concentration of a second metabolite. The output is a current signal. Further, the tip portion may have three grooves for the first sensor electrode, second sensor electrode and reference electrode. The first metabolite may be glucose and the second metabolite may be lactate.

The sensor may further include an elongated body portion having a body axis extending centrally through the elongated body portion and a tip portion having a tip axis extending centrally through the tip portion. The tip portion includes the substrate (and sensor electrode(s) and sensing layer(s)). The tip portion is disposed adjacent the elongated body portion and at an obtuse angle of between 90° to 170°, and more preferably between 130° to 160° between the body axis and the tip axis.

This arrangement (i.e. the tip portion is disposed adjacent the elongated body portion and at an obtuse angle of between 90° to 170°) advantageously facilitates tissue penetration compared to an elongated sensor device without any bent portion(s).

The elongated body portion may include a fluid reservoir and the tip portion may further include an inflatable member. A channel is disposed between the fluid reservoir and the inflatable member to provide fluid communication between the fluid reservoir and the inflatable member.

The elongated body portion may further include an actuator configured to deliver a volume of fluid in the fluid reservoir through the channel to the inflatable member to inflate the inflatable member. The inflatable member advantageously allows for stitch-less securement and easy removal of the sensor to both surface and buried tissues such as flaps. For example, after insertion of the sensor into a patient's body, the inflatable member can be continuously inflated to provide a tighter fit in the patient's body to better secure the sensor to the patient's body. In other words, the inflatable member is designed to expand outward so that the sensor can be held in place within the patient's body by frictional contact between the inflated member and tissue. During removal of the sensor, the inflatable member can be further inflated to temporarily displace the tissue around the sensor to facilitate removal of the sensor. Alternatively, during removal of the sensor, the inflatable member can be deflated to facilitate removal of the sensor. The fluid may be an incompressible fluid such as saline solution which is filled up within the reservoir chamber. The inflatable member may be made of an elastomer.

The shape of the sensor and the size of the sensor are similar to that of a needle in order for the sensor to be easily implanted in the tissue. The sensor component can be needle-like, micro-needlelike, elongated, with an angular or curved edge or any other suitable structural or mechanical format which allows its insertion into the patient's skin. In one embodiment, the size is approximately 5 mm (L) by 2.3 mm (W) by 0.85 mm (H). These dimensions are non-limiting.

The elongated body portion acts as a relay in order to easily connect wires to the tip portion having the sensing elements. The shape and size of the elongated body portion may vary depending on usage. The angular incline between the elongated body portion and the sensor component also facilitates for easier implantation, removal and securement of the sensor into/from/onto the tissue. The tip portion may be tapered to facilitate tissue penetration.

The substrate functions as a physical support for the first sensor electrode, second sensor electrode and reference electrode. The substrate also protects the first sensor electrode, second sensor electrode and reference electrode, provides consistency of electrode size, polymer depth, and preserves inter-electrode distance. An electrical connection may be established between all relevant components.

A current signal receiver/detector may be provided, together with a processor module configured to execute a processing algorithm. The input is the sensor output in Amperes and time. The output is a tissue failure risk score and an alert.

In an implementation, there is provided a protective layer (eg. Nafion, poly(ethylene glycol) (PEG), PEG derived hydrogel, polyethylene oxide, polyurethanes, biomimicry, silicone elastomers, porous carbon coating) over the sensor polymer/enzyme layer for protection from environmental factors.

The working temperature range of the sensor is expected to be from room temperature to about 45° C., and minimum detectable change of metabolite level is as low as 0.2 mM.

Bio-active centers of enzymes are surrounded by a thick protein layer and are located deeply in hydrophobic cavity of molecules. The direct electrochemistry or electron transfer within enzyme is therefore difficult. Therefore, the use of an electrical connector is required to enhance the transportation of electrons between enzyme and the metabolites. A redox polymer or an enzyme-containing polymer may be used as a redox centre to mediate the electron transfer from enzyme to electrode due to its advantages including fast electron transfer rate, high current density, good biocompatibility, good chemical stability, and inertness to microbial degradation. Attachment of enzyme to the electrode can be achieved by methods such as adsorption, encapsulation, entrapment, covalent binding, cross-linking and so on. Among them, chemical cross-linking provides good long term stability. The enzyme-containing polymer is fabricated onto the electrode surface. A layer of Nafion may then be optionally coated on the sensing layer for longer stability. The resulting sensor may be applied both in-vitro and in-vivo. The currents produced by the electro-oxidation of glucose and/or lactate by their enzymes are measured by a potentiostat.

The sensor as defined above, wherein the enzyme-containing polymer may have a thickness in the range of about 0.010 mm to about 0.200 mm, about 0.020 mm to about 0.200 mm, about 0.030 mm to about 0.200 mm, about 0.040 mm to about 0.200 mm, about 0.050 mm to about 0.200 mm, about 0.060 mm to about 0.200 mm, about about 0.070 mm to about 0.200 mm, about 0.080 mm to about 0.200 mm, about 0.090 mm to about 0.200 mm, about 0.100 mm to about 0.200 mm, about 0.110 mm to about 0.200 mm, about 0.120 mm to about 0.200 mm, about 0.130 mm to about 0.200 mm, about 0.140 mm to about 0.200 mm, about 0.150 mm to about 0.200 mm, about 0.160 mm to about 0.200 mm, about 0.170 mm to about 0.200 mm, about 0.180 mm to about 0.200 mm, about 0.190 mm to about 0.200 mm, about 0.010 mm to about 0.020 mm, about 0.010 mm to about 0.030 mm, about 0.010 mm to about 0.040 mm, about 0.010 mm to about 0.050 mm, about 0.010 mm to about 0.060 mm, about 0.010 mm to about 0.070 mm, about 0.010 mm to about 0.080 mm, about 0.010 mm to about 0.090 mm, about 0.010 mm to about 0.100 mm, about 0.010 mm to about 0.110 mm, about 0.010 mm to about 0.120 mm, about 0.010 mm to about 0.130 mm, about 0.010 mm to about 0.140 mm, about 0.010 mm to about 0.150 mm, about 0.010 mm to about 0.160 mm, about 0.010 mm to about 0.170 mm, about 0.010 mm to about 0.180 mm, about 0.010 mm to about 0.190 mm or preferably about 0.125 mm.

In this regard, it should be noted that the invention may also cover alternative methods of achieving the chemistry of this technology. In other words, methods which allow for the scale-up and production of the components of the device should be contemplated as relevant to this technology.

An in-vitro electrochemical performance study was performed with a CHI1040C Potentiostat (CH instruments, Austin, Tex.). The amperometric response of the sensor to different concentration of metabolites (1 mM to 30 mM in PBS buffer) at 0.3 V was recorded. The continuous monitoring of metabolites was studied up to 5.5 days.

In an alternative embodiment, the electrochemical metabolites biosensor described may be industrially fabricated as follows:

(1) Fabricate the PC chips (3 layers) and gold foil by milling and wirecutting machining respectively.

(2) Bond layer 1 (PC 0.5 mm) and layer 2 (PC 0.25 mm).

(3) Screen print Ag trace to the bonded layer 2. Cure at 130 Degree Celsius inside oven.

(4) Protect Ag trace with Mylar film.

(5) Embed the gold trace to the bonded layer 2.

(6) Remove Mylar film, Bond layer 3 (0.1 mm).

(7) Protect gold trace with Mylar film.

(8) Screen print Ag/Cl onto Ag trace

(9) Dispense polymer with syringe pump at 1 ul/min for 5 second. Cure the polymer.

(10) Repeat step 9 for 2 times until polymer reaches 0.1 mm thickness.

It should also be contemplated that alternative materials or chemical components may be used for the specific components and not limited to the specific examples provided above.

In yet another embodiment, a membrane incorporated with enzymes may be able to amplify signal and filter irrelevant analytes.

Wires connect the sensor to a reader/analyser. The wires may be insulated electrical wires connecting the sensors to the reader/analyzer. For buried tissues cases, the wires may follow drain cannulas that are usually present for draining out fluids. These wires may be used to transfer an electrical signal coming from the sensor to the reader. These electrical signals are read and interpreted by the reader. In an alternative embodiment, the connection may be a wireless connection. In such an embodiment, the wires may be replaced by means such as Near-Field Communication (NFC), which makes the entire device wireless and therefore potentially more convenient during use. The wireless components may be embedded in the elongated body portion.

The reader/analyzer may be a potentiostat that is used to collect, analyse and compare current readings (in Amperes) between the control sensor and tissue sensor. At set intervals (e.g. every 15 minutes); the reader/analyzer collects these data and evaluates the trend. For example, if the results from both sensors are following the same upward trend, the reader/analyzer shows that the tissue is still healthy. However, if the readings from the tissue sensor deviate significantly from the control sensor, an algorithm computes the probability that the tissue is failing. If the tissue is detected to be failing, clinicians are alerted as surgical intervention may be needed. The algorithm may be present in the reader/analyzer.

The reader/analyzer may comprise three (3) main parts: a reader, a processor that executes an analysing algorithm and an alert system. The reader may be a potentiostat, which measures the outputs of the sensors in Amperes and plots a graph of those outputs. The graphs show a measure of Amperes over time and may be interpreted as a concentration level of glucose and lactate of the tissue at a time when the measurements are being taken. As such, four (4) different readings may be collected at a point in time: two (2) readings from the control sensor and two (2) from the tissue sensor. The two (2) readings from the sensor correspond to 1) the glucose concentration level and 2) the lactate concentration level. The lactate levels may be determined by detecting current when lactate is oxidized by its enzyme. The detection algorithm for tissue viability may be based on glucose and lactate levels.

The readings in Amperes over time are then analyzed using an algorithm. The algorithm may compare the glucose concentration level from the control sensor with the one from the tissue sensor, and perform the same comparison for the lactate concentration level. After which, it may be able to evaluate the glucose and lactate trends coming from both sensors.

Four (4) possible illustrations may arise from the analysis as provided below, although others are also contemplated. It should also be contemplated that alternative metabolites or chemical components could be used and therefore these would provide alternative illustrations of readings which can provide a reference to study the tissue failure condition.

Illustrations:

(1) The glucose and lactate concentrations for both sensors trend in a similar way.

(2) The glucose concentration from the tissue falls below a certain threshold as compared to the glucose concentration from the control value, but the lactate concentration from both sensors still trend in the same direction.

(3) The lactate concentration from the tissue increases above a certain threshold as compared to the lactate concentration from the control value, but the glucose concentration from both sensors still trend in the same direction.

(4) The glucose concentration from the tissue falls below a certain threshold as compared to the glucose concentration from the control value and the lactate concentration from the tissue increases above a certain threshold as compared to the lactate concentration from the control value.

In the first situation for example, the tissue's metabolite levels are increasing and decreasing in a similar fashion as the metabolites sensed at the control sensor, therefore the tissue is considered to be healthy.

In the second and third situations for example, the tissue's glucose or lactate level is not consistent with the natural state or the resting state of a healthy or failing tissue. In such cases, a second critical threshold may be implemented for the glucose and lactate levels. If the lactate rises above this threshold or if the glucose drops below this threshold, the tissue is considered to be failing.

In the fourth situation for example, the glucose level from the tissue has decreased too much as compared to the glucose sensed at the control sensor and the lactate level has increased. Therefore the tissue is considered to be failing.

The above are illustrations only and other situations or examples may be envisaged.

The different thresholds may be based on relative percentage differences between the tissue's metabolite levels and the control metabolite levels. However, other types of information, characteristics or threshold differences may also be relevant for the workings of this technology in order to provide an insight into a patient's tissue failure condition. The other types of relevant thresholds would be in line with a control value of an appropriate algorithm that could provide details of the patient's tissue failure condition. Therefore, while the differences may be based on relative percentage differences, alternative scenarios are also possible.

The reader/analyzer may include an alarm system to alert the clinical team that the patient's tissue is failing. If one or more critical thresholds are reached, an alarm or an alternative signalling or notification system may be triggered in accordance with an appropriate algorithm which provides a quantification of a tissue failure effect.

The system may serve as a platform technology that could be used for other purposes such as, for example, monitoring tissue perfusion status in transplanted organs (kidney, liver, lung, heart, face, hand, and reproductive organs). Studies have shown that measurement of analyte concentration like glucose or lactate can improve the chances of detecting early signs of ischemia thus improving patient care in those areas. Moreover, studies have also shown that lactate is an important aspect when it comes to monitoring critically ill patients. Indeed, when these patients are in the early resuscitation phase, the treatment choice may be determined by knowing their level of blood lactate. The monitoring system may therefore be implemented for this need.

Advantageously, the embodiments of the invention seek to detect failing tissues earlier and objectively, provide direct monitoring of a targeted organ, save clinician time monitoring each patient, and provide saving on costs arising from complications.

Further, unless the context clearly requires otherwise, throughout the description and the claims, the words “comprise”, “comprising” and the like are to be construed in an inclusive as opposed to an exclusive or exhaustive sense; that is to say, in the sense of “including, but not limited to”.

### BRIEF DESCRIPTION OF DRAWINGS

The accompanying drawings illustrate a disclosed embodiment and serves to explain the principles of the disclosed embodiment. It is to be understood, however, that the drawings are designed for purposes of illustration only, and not as a definition of the limits of the invention.

FIG. 1 shows a schematic diagram of the coupling redox reaction which occurs in a redox polymer mediated sensor.

FIG. 2 shows a schematic diagram of a sensor operating in a deflated mode, according to an example embodiment.

FIG. 3 shows a schematic diagram of a sensor operating in an inflated mode, according to an example embodiment.

FIG. 4 shows a schematic diagram (zoomed-in isometric view) of a sensor operating in a deflated mode, according to an example embodiment.

FIG. 5 shows a schematic diagram (zoomed-in isometric view) of a sensor operating in an inflated mode, according to an example embodiment.

FIG. 6 shows a schematic diagram (zoomed-in side view) of a sensor operating in a deflated mode, according to an example embodiment.

FIG. 7 shows a schematic diagram (zoomed-in side view) of a sensor operating in an inflated mode, according to an example embodiment.

FIG. 8 shows a schematic diagram (zoomed-in top view) of a sensor operating in a deflated mode, according to an example embodiment.

FIG. 9 shows a schematic diagram (zoomed-in bottom view) of a sensor operating in a deflated mode, according to an example embodiment.

FIG. 10A shows a schematic diagram (zoomed-in side view) of a sensor with a fluid reservoir operating in a deflated mode, according to an example embodiment.

FIG. 10B shows a schematic diagram (zoomed-in side view) of a sensor with a fluid reservoir operating in an inflated mode, according to an example embodiment.

FIG. 11 shows a schematic diagram of a sensor (zoomed-in exploded isometric view), according to an example embodiment.

FIG. 12A shows a schematic diagram of a tip portion (exploded isometric view), according to an example embodiment.

FIG. 12B shows a schematic diagram of a tip portion (isometric view), according to an example embodiment.

FIG. 13A shows a schematic diagram of a sensor operating in an activated mode, according to an example embodiment.

FIG. 13B shows a schematic diagram (zoomed-in isometric view) of a sensor operating in an activated mode, according to an example embodiment.

FIG. 13C shows a schematic diagram (zoomed-in side view) of a sensor operating in an activated mode, according to an example embodiment.

FIG. 13D shows a schematic diagram (zoomed-in isometric view) of a sensor operating in a deactivated mode, according to an example embodiment.

FIG. 13E shows a schematic diagram (zoomed-in side view) of a sensor operating in a deactivated mode, according to an example embodiment.

FIG. 13F shows a schematic diagram (zoomed-in isometric view) of a sensor in an intermediate mode, according to an example embodiment.

FIG. 13G shows a schematic diagram (zoomed-in side view) of a sensor in an intermediate mode, according to an example embodiment.

FIG. 14A shows a schematic diagram (zoomed-in isometric view) of a sensor, according to an example embodiment.

FIG. 14B shows a schematic diagram (zoomed-in top view) of a sensor, according to an example embodiment.

FIG. 14C shows a schematic diagram (zoomed-in side view) of a sensor, according to an example embodiment.

FIG. 14D shows a schematic diagram (zoomed-in back view) of a sensor, according to an example embodiment.

FIG. 15 shows a change in the current signal upon the dilution of 1 mM of glucose with a phosphate buffer solution.

FIG. 16 shows a change in the current signal upon the continuous addition of 100 μL of 2 mM lactate into 10 mL of phosphate buffer solution (PBS) under stirring.

FIG. 17 shows the structure of chitosan-ferrocenyl/branched polyethylenimine-ferrocenyl (CHIT-Fc/BPEI-Fc) conjugate whereby the chitosan-ferrocenyl (CHIT-Fc) redox polymer was cross-linked with branched polyethylenimine-ferrocenyl (BPEI-Fc) intermediate via a cross-linker (1701), glutaraldehyde (GA).

FIG. 18 shows the enzyme-containing polymer whereby the enzyme may be a glucose oxidase (1801) or lactate oxidase (1803) and the cross-linker (1805).

FIG. 19 shows a voltammogram of the chitosan-ferrocenyl/branched polyethylenimine-ferrocenyl (CHIT-Fc/BPEI-Fc) conjugate where the current at the working electrode is plotted versus the applied voltage (that is, the working electrode's potential) to give the cyclic voltammogram trace.

FIG. 20 shows an amperometric measurement graph (current over time) to record the current change over time, for the enzyme-containing polymer (glucose oxidase), whereby the sensitivity on glucose was tested. The volume ratio between chitosan-ferrocenyl/branched polyethylenimine-ferrocenyl (CHIT-Fc/BPEI-Fc) conjugate and glucose oxidase is 3:1.

FIG. 21 shows an amperometric measurement graph (current over time) to record the current change over time, for the enzyme-containing polymer (glucose oxidase), whereby the sensitivity on glucose was tested. The volume ratio between chitosan-ferrocenyl/branched polyethylenimine-ferrocenyl (CHIT-Fc/BPEI-Fc) conjugate and glucose oxidase is 3:2.

FIG. 22 shows an amperometric measurement graph (current over time) to record the current change over time, for the enzyme-containing polymer (lactate oxidase), whereby the sensitivity on lactate was tested. The volume ratio between chitosan-ferrocenyl/branched polyethylenimine-ferrocenyl (CHIT-Fc/BPEI-Fc) conjugate and lactate oxidase is 3:1.

FIG. 23 shows an amperometric measurement graph (current over time) to record the current change over time, for the enzyme-containing polymer (lactate oxidase), whereby the sensitivity on lactate was tested. The volume ratio between chitosan-ferrocenyl/branched polyethylenimine-ferrocenyl (CHIT-Fc/BPEI-Fc) conjugate and lactate oxidase is 3:2.

FIG. 24 shows a series of photographs of the animal (rabbit) test for the glucose and lactate measurements using the sensor chip coated with the enzyme-containing polymer: FIG. 24A shows how the flap (2401) was raised and removed from the rabbit, FIG. 24B and FIG. 24C show the locations of the blood vessels supplying to the muscle and skin (2411 and 2421), FIG. 24D shows the sensor chip (2433) being wrapped around with the flap (2431), and FIG. 24E shows the blood vessel being clamped (2441) for the test.

FIG. 25 shows an amperometric measurement graph (current over time) to record the current change over time for the animal test on glucose (2501), before and after clamping (2503) of the blood vessel on the flap, at 600 seconds.

FIG. 26 shows an amperometric measurement graph (current over time) to record the current change over time for the animal test on lactate (2601), before and after clamping (2603) of the blood vessel on the flap, at 600 seconds.

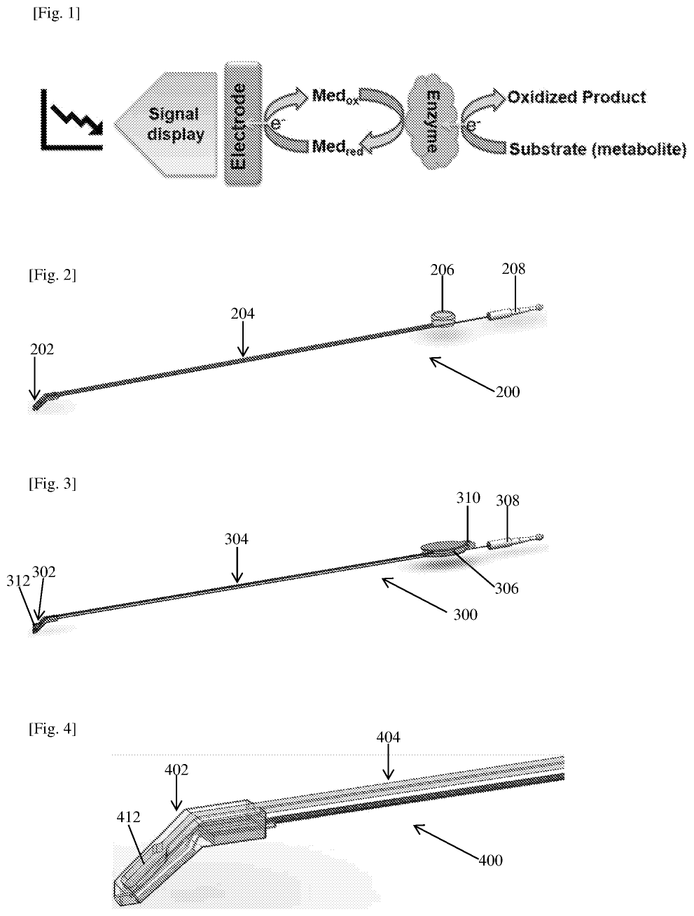

### DETAILED DESCRIPTION OF DRAWINGS

Referring to FIG. 2, FIG. 2 shows a schematic diagram of a sensor operating in a deflated mode, according to an example embodiment. The sensor 200 includes an elongated body portion 204 and a tip portion 202. The elongated body portion 204 includes a fluid reservoir 206. The sensor 200 further includes a connector 208. The connector 208 includes an interface for connection to a reader, a display module, or a monitor device.

Referring to FIG. 3, FIG. 3 shows a schematic diagram of a sensor operating in an inflated mode, according to an example embodiment. Similar to FIG. 2, the sensor 300 includes an elongated body portion 304 and a tip portion 302. The tip portion 302 further includes an inflatable member 312. A channel is disposed between the fluid reservoir 306 and the inflatable member 312 to provide fluid communication therebetween. The fluid reservoir 306 contains an incompressible fluid and non-toxic substance such as saline solution. An actuator 310 (shown in FIG. 3 as a clipping mechanism) is configured to deliver a volume of fluid in the fluid reservoir 306 through the channel to the inflatable member 312 to inflate the inflatable member. Comparing FIG. 2 and FIG. 3, it can be seen in FIG. 3 that the inflatable member 312 is inflated when the actuator 310 is disposed over the fluid reservoir 306. The actuator 310 provides a compressive force to move the incompressible fluid from the fluid reservoir 306 through the channel to the inflatable member 312.

Referring to FIG. 4, FIG. 4 shows a schematic diagram (zoomed-in isometric view) of a sensor operating in a deflated mode, according to an example embodiment. Similar to FIGS. 2 and 3, the sensor 400 includes an elongated body portion 404 and a tip portion 402. The tip portion 402 includes an inflatable member 412 that is deflated.

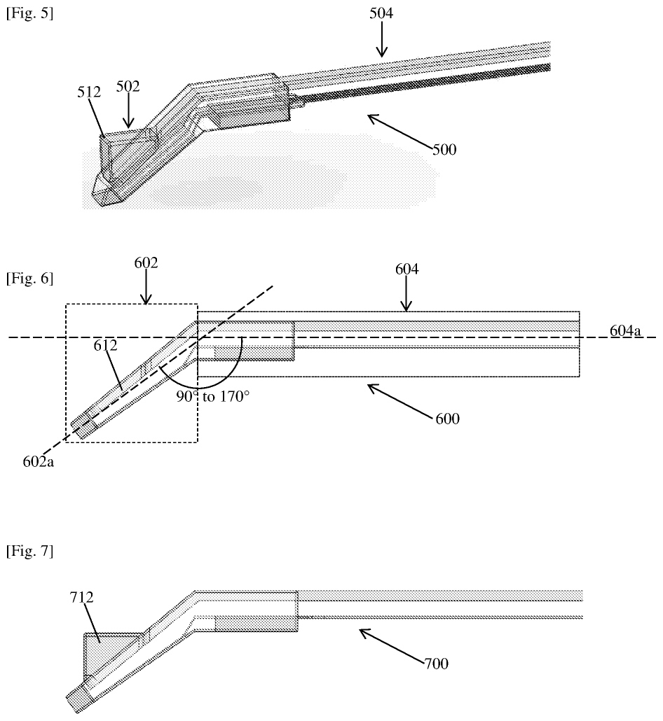

Referring to FIG. 5, FIG. 5 shows a schematic diagram (zoomed-in isometric view) of a sensor operating in an inflated mode, according to an example embodiment. Similar to FIG. 5, the sensor 500 includes an elongated body portion 504 and a tip portion 502. The tip portion 502 includes an inflatable member 512 that is inflated.

Referring to FIG. 6, FIG. 6 shows a schematic diagram (zoomed-in side view) of a sensor operating in a deflated mode, according to an example embodiment. The sensor 600 includes an elongated body portion (clearly demarcated by dashed region 604) and a tip portion (clearly demarcated by dashed region 602). The tip portion 602 includes an inflatable member 612 that is deflated. The elongated body portion 604 has a body axis 604a extending centrally through the elongated body portion 604. The tip portion 602 has a tip axis 602a extending centrally through the tip portion 602. The tip portion 602 is disposed adjacent the elongated body portion 604 and at an obtuse angle of between 90° to 170° between the body axis 604a and the tip axis 602a. More preferably, the obtuse angle between the body axis 604a and the tip axis 602a is about 130° to 160°.

Referring to FIG. 7, FIG. 7 shows a schematic diagram (zoomed-in side view) of a sensor operating in an inflated mode, according to an example embodiment. The sensor 700 is similar to sensor 600, and show an inflatable member 712 that is inflated.

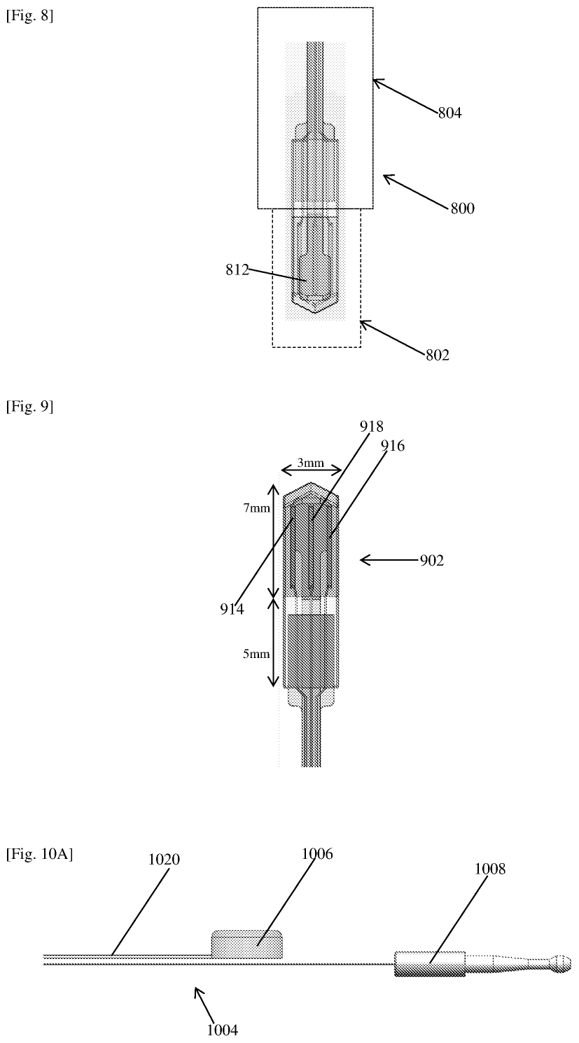

Referring to FIG. 8, FIG. 8 shows a schematic diagram (zoomed-in top view) of a sensor operating in a deflated mode, according to an example embodiment. Similar to FIGS. 2 to 7, the sensor 800 includes an elongated body portion (clearly demarcated by dashed region 804) and a tip portion (clearly demarcated by dashed region 802). The tip portion 802 includes an inflatable member 812 that is deflated.

Referring to FIG. 9, FIG. 9 shows a schematic diagram (zoomed-in bottom view) of a sensor operating in a deflated mode, according to an example embodiment. The tip portion 902 of the sensor comprises a substrate (not clearly seen in FIG. 9). A first sensor electrode 914, a second sensor electrode 916 and a reference electrode 918 are disposed on the substrate. The first sensor electrode 914 and the second sensor electrode 916 may comprise gold or carbon. The reference electrode 918 may comprise silver (Ag) or silver chloride (AgCl).

Referring to FIG. 10A, FIG. 10A shows a schematic diagram (zoomed-in side view) of a sensor with a fluid reservoir operating in a deflated mode, according to an example embodiment. Similar to FIG. 3, the sensor includes an elongated body portion 1004. A channel 1020 is disposed between the fluid reservoir 1006 and an inflatable member (not shown) to provide fluid communication therebetween. The fluid reservoir 1006 contains an incompressible fluid and non-toxic substance such as saline solution. The sensor further includes a connector 1008. The connector 1008 includes an interface for connection to a reader, a display module, or a monitor device (not shown).

Referring to FIG. 10B, FIG. 10B shows a schematic diagram (zoomed-in side view) of a sensor with a fluid reservoir operating in an inflated mode, according to an example embodiment. Similar to FIG. 10A, the sensor includes an elongated body portion 1004. A channel 1020 is disposed between the fluid reservoir 1006 and an inflatable member (not shown) to provide fluid communication therebetween. The fluid reservoir 1006 contains an incompressible fluid and non-toxic substance such as saline solution. The sensor further includes a connector 1008. An actuator 1010 (shown in FIG. 10B as a clipping mechanism) is configured to deliver a volume of fluid in the fluid reservoir 1006 through the channel 1020 to the inflatable member to inflate the inflatable member. When the actuator 1010 is disposed over the fluid reservoir 1006, the fluid reservoir 1006 (which is made from a resilient and flexible material) is compressed. In other words, the actuator 1010 provides a compressive force to move the incompressible fluid from the fluid reservoir 1006 through the channel 1020 to the inflatable member.

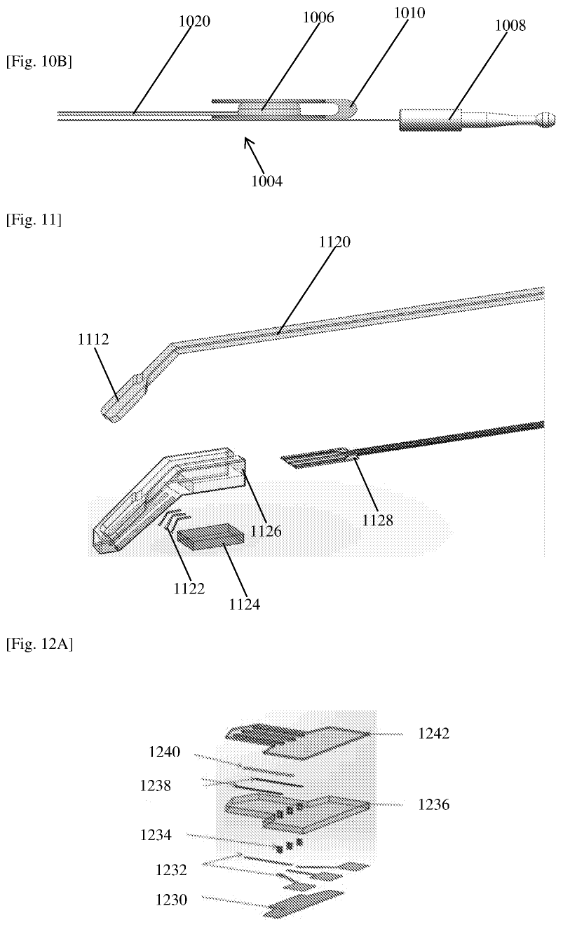

Referring to FIG. 11, FIG. 11 shows a schematic diagram of a sensor (zoomed-in exploded isometric view), according to an example embodiment. The sensor comprises a channel 1120 that is in fluid communication with inflatable member 1112. The sensor also includes a sensor body 1126 that may be made of polycarbonate. The inflatable member 1112 and a portion of the channel 1120 can be disposed within the sensor body 1126. A flexible electronics wire film 1128 provides electrical connection between electrodes at the tip portion and a reader or monitor device. An electronic wiring strain relief 1124 with integrated dielectric film may be provided.

Silver vias with silver trace 1122 may also be provided for formation of electrodes. The electrodes may be disposed within grooves formed in the sensor body 1126. The strain relief 1124 prevents the silver vias with silver trace connections 1122 from wear and tear and loss of connection when the wires 1128 are bent during insertion, securement or removal of the sensor body. The dielectric act as a medium of connection to the wires 1128 as well as insulation and protection of the silver vias with silver trace connections 1122 from external elements such as blood or other bodily fluids when the sensor is inserted, secured or removed.

Referring to FIG. 12A, FIG. 12A shows a schematic diagram of a tip portion (exploded isometric view), according to an example embodiment. A substrate 1230 provides physical support and protects the sensor electrodes, provides consistency of electrode size, polymer depth and preserves inter-electrode distance. Otherwise, sensor performance may be inconsistent and the sensors may rapidly degrade through environmental exposure. Silver electrodes and trace 1232 are provided. Silver vias 1234 are provided to enable metal contact. A polycarbonate layer 1236 may be provided. The polycarbonate layer 1236 may be about 0.5 mm thick. Furthermore, carbon electrodes 1238 and/or gold electrodes 1240 may be provided. Finally, another polycarbonate layer 1242 may be provided. The polycarbonate layer 1242 may be about 0.125 mm thick.

Referring back to FIG. 9, the carbon electrodes 1238 and/or gold electrodes 1240 correspond to the first sensor electrode 914 and/or the second sensor electrode 916. The silver electrode 1232 corresponds to the reference electrode 918.

A first sensing layer is disposed on the first sensor electrode 914. The first sensing layer comprises a first enzyme-containing polymer as described herein. A second sensing layer is disposed on the second sensor electrode 916. The second sensing layer comprises a second enzyme-containing polymer as described herein. The enzyme-containing polymer has a thickness of in the range of about 0.010 mm to about 300 mm. The enzyme-containing polymer can be deposited above carbon electrodes 1238 within the confines of the slits cut-out in the polycarbonate layer 1242.

Referring to FIG. 12B, FIG. 12B shows a schematic diagram of a tip portion (isometric view), according to an example embodiment. Similar to FIG. 12A, a substrate 1230 is provided. Silver electrodes and trace 1232 are provided. A first lower polycarbonate layer 1236 may be provided. Carbon electrodes 1238 may be provided. A second upper polycarbonate layer 1242 may be provided. The enzyme-containing polymer can be deposited above carbon electrodes 1238 within the confines of the slits cut-out in the second upper polycarbonate layer 1242.

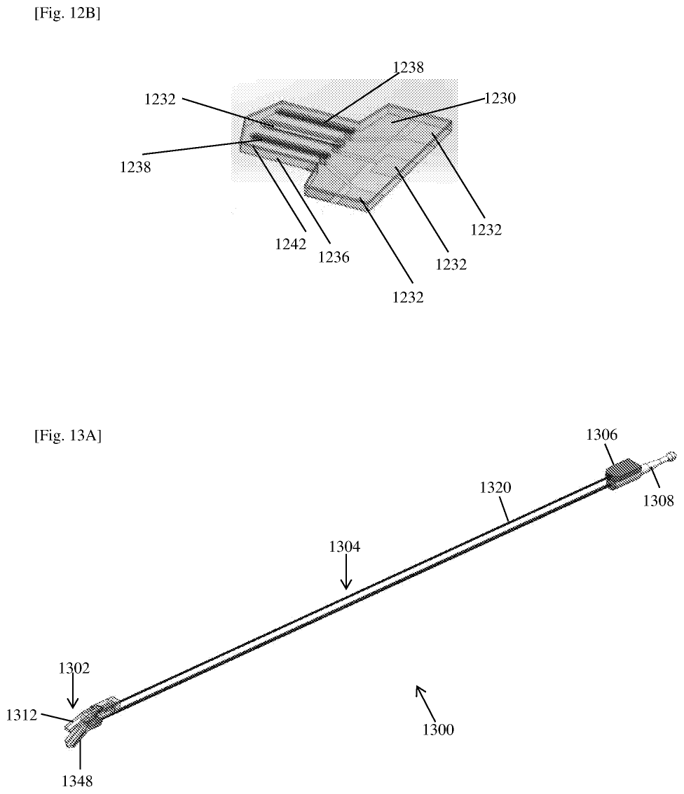

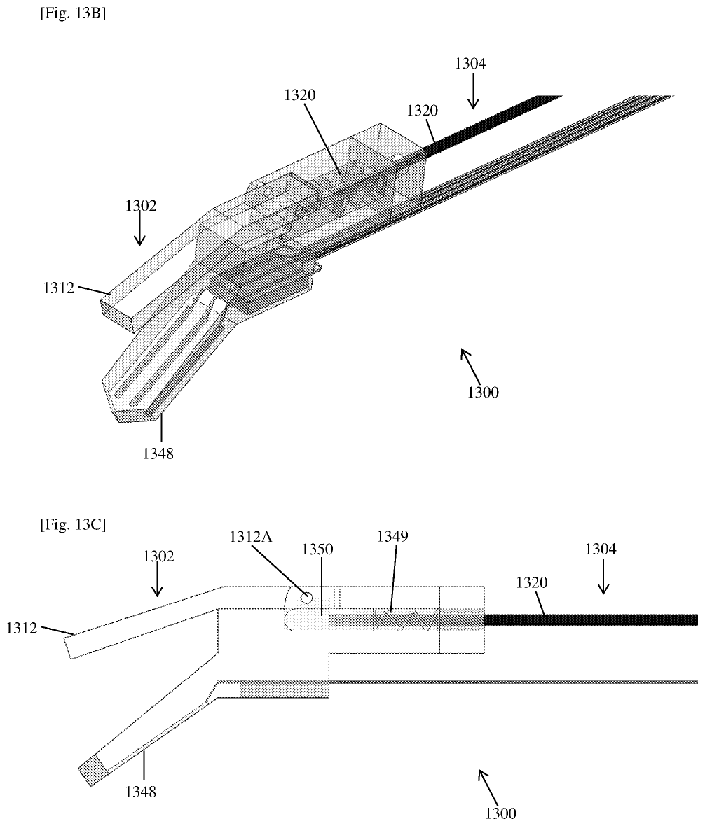

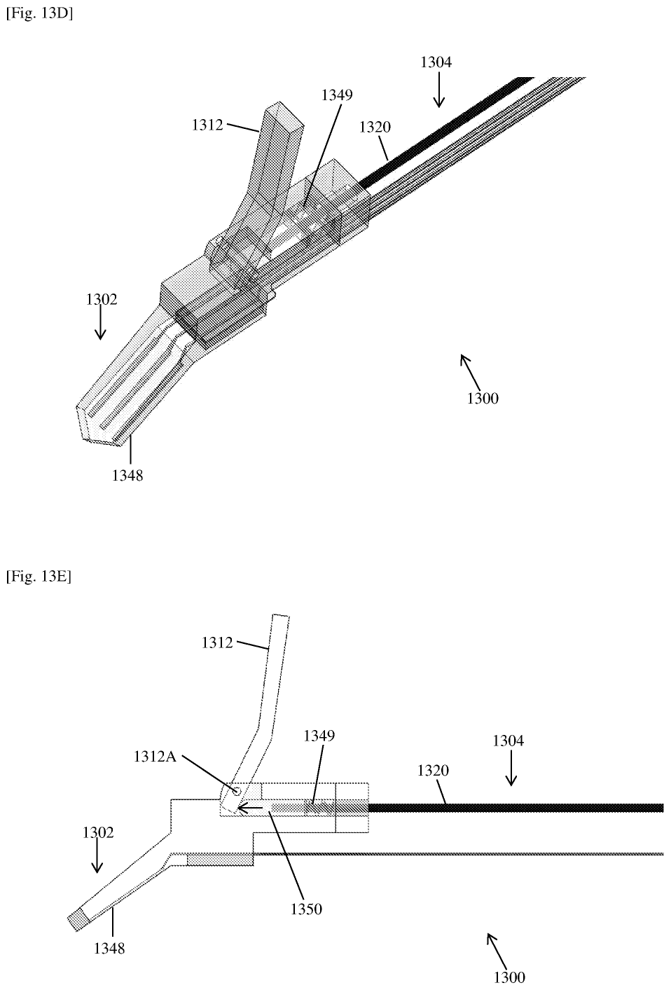

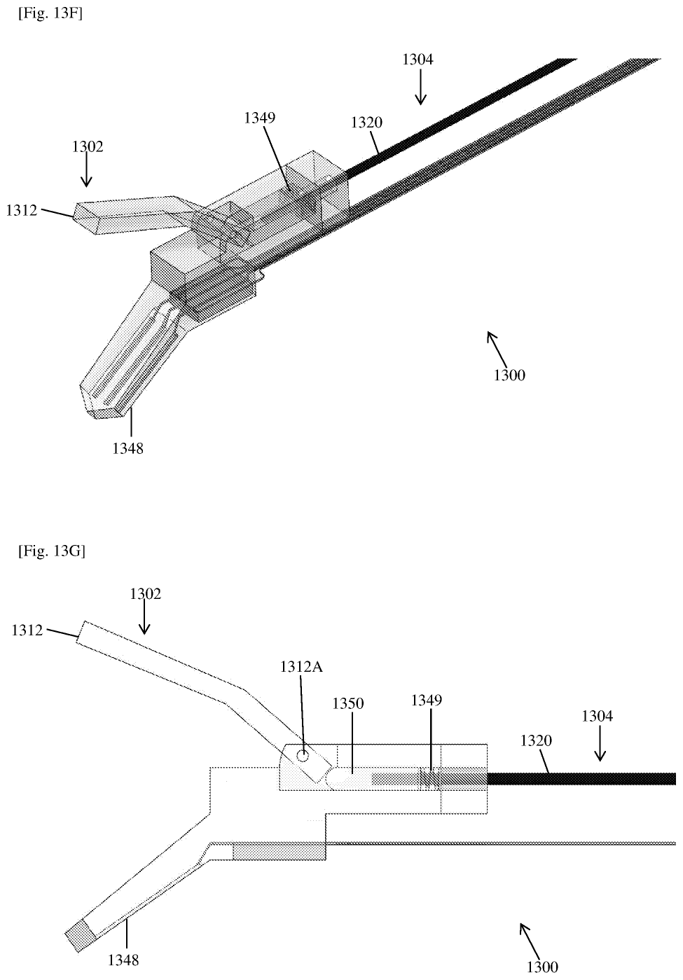

Referring to FIG. 13A, FIG. 13A shows a schematic diagram of a sensor operating in an activated mode, according to an example embodiment. Similar to FIG. 2, the sensor 1300 includes an elongated body portion 1304 and a tip portion 1302. The elongated body portion 1304 includes an actuating arm 1306, a cable extension 1320, a biasing member 1349 and a securing pin 1350 (shown in FIG. 13C). The actuating arm 1306 is operatively coupled to the cable extension 1320, which is in turn operatively coupled to the biasing member 1349 and the securing pin 1350. The tip portion 1302 includes a moveable member 1312 and a fixed member 1348. The fixed member 1348 includes the first sensor electrode 914, the second sensor electrode 916 and the reference electrode 918 as shown in FIG. 9. Similar to FIG. 2, the sensor 1300 further includes a connector 1308. The connector 1308 includes an interface for connection to a reader, a display module, or a monitor device.

Referring to FIG. 13B, FIG. 13B shows a schematic diagram (zoomed-in isometric view) of a sensor operating in an activated mode, according to an example embodiment. The tip portion 1302 includes the fixed member 1348 and the moveable member 1312 that is in the activated mode.

Referring to FIG. 13C, FIG. 13C shows a schematic diagram (zoomed-in side view) of a sensor operating in an activated mode, according to an example embodiment. The tip portion 1302 includes the fixed member 1348 and the moveable member 1312 that is in the activated mode. The elongated body portion 1304 includes the actuating arm 1306 (not shown in FIG. 13C), the cable extension 1320, the biasing member 1349 that is in the activated mode and the securing pin 1350 that is in the activated mode.

Referring to FIG. 13D, FIG. 13D shows a schematic diagram (zoomed-in isometric view) of a sensor operating in a deactivated mode, according to an example embodiment. The tip portion 1302 includes the fixed member 1348 and the moveable member 1312 that is in the deactivated mode.

Referring to FIG. 13E, FIG. 13E shows a schematic diagram (zoomed-in side view) of a sensor operating in a deactivated mode, according to an example embodiment. The tip portion 1302 includes the fixed member 1348 and the moveable member 1312 that is in the deactivated mode. The elongated body portion 1304 includes the actuating arm 1306 (not shown in FIG. 13E), the cable extension 1320, the biasing member 1349 that is in the deactivated mode and the securing pin 1350 that is in the deactivated mode.

Referring to FIG. 13F, FIG. 13F shows a schematic diagram (zoomed-in isometric view) of a sensor in an intermediate mode, according to an example embodiment. The tip portion 1302 includes the fixed member 1348 and the moveable member 1312 that is in the intermediate mode between the activated mode and the deactivated mode.

Referring to FIG. 13G, FIG. 13G shows a schematic diagram (zoomed-in side view) of a sensor in an intermediate mode, according to an example embodiment. The tip portion 1302 includes the fixed member 1348 and the moveable member 1312 that is in the intermediate mode. The elongated body portion 1304 includes the actuating arm 1306 (not shown in FIG. 13G), the cable extension 1320, the biasing member 1349 that is in the intermediate mode and the securing pin 1350 that is in the intermediate mode.

In use, sensor 1300 in the deactivated mode is placed at a tissue such that the fixed member 1348 of the sensor 1300 is within the tissue and the moveable member 1312 is outside a patient's body. To secure the sensor 1300 to the patient, the moveable member 1312 is physically pressed downwards by a user such that the moveable member 1312 rotates about a pivot 1312A to the activated position. Referring to FIG. 13C, the biasing member 1349 that is in the form of a spring in this embodiment, provides a resilient force on the securing pin 1350 and causes the securing pin 1350 to extend fully in the activated mode, thereby securing the moveable member 1312 at the activated position by restricting the moveable member 1312 from rotating about the pivot 1312A.

To remove the sensor 1300 from the tissue, the actuating arm 1306 is pulled to retract the cable extension 1320, which in turn causes the biasing member 1349 to compress and the securing pin 1350 to slide along guide tracks to the deactivated position, such that the moveable member 1312 can rotate about the pivot 1312A to the deactivated position when the moveable member 1312 is physically pulled upwards by the user. Referring to FIG. 13E, the biasing member 1349 provides a resilient force on the securing pin 1350 and causes the securing pin 1350 to exert a force (see arrow at FIG. 13E) on the moveable member 1312 in the activated mode, thereby securing the moveable member 1312 at the activated position by restricting the moveable member 1312 from rotating about the pivot 1312A.

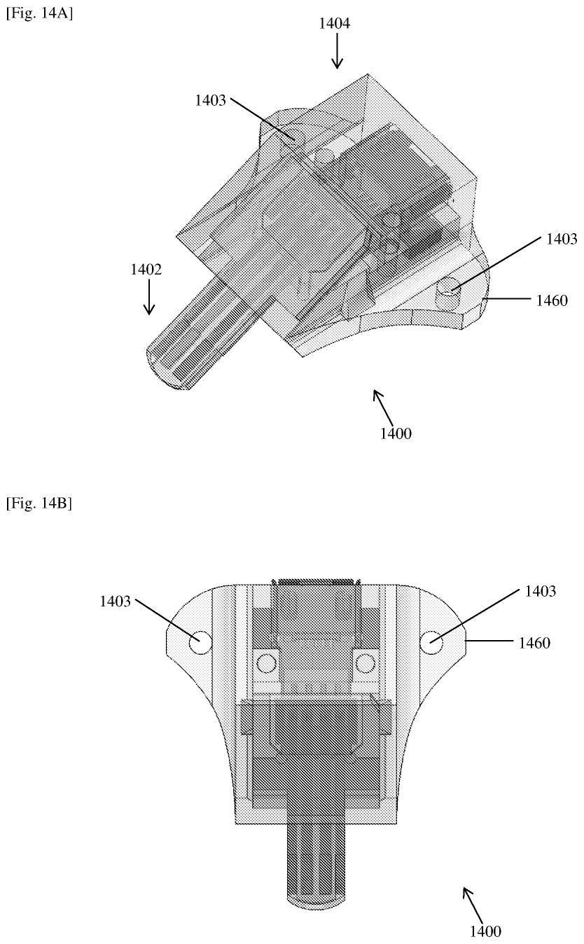

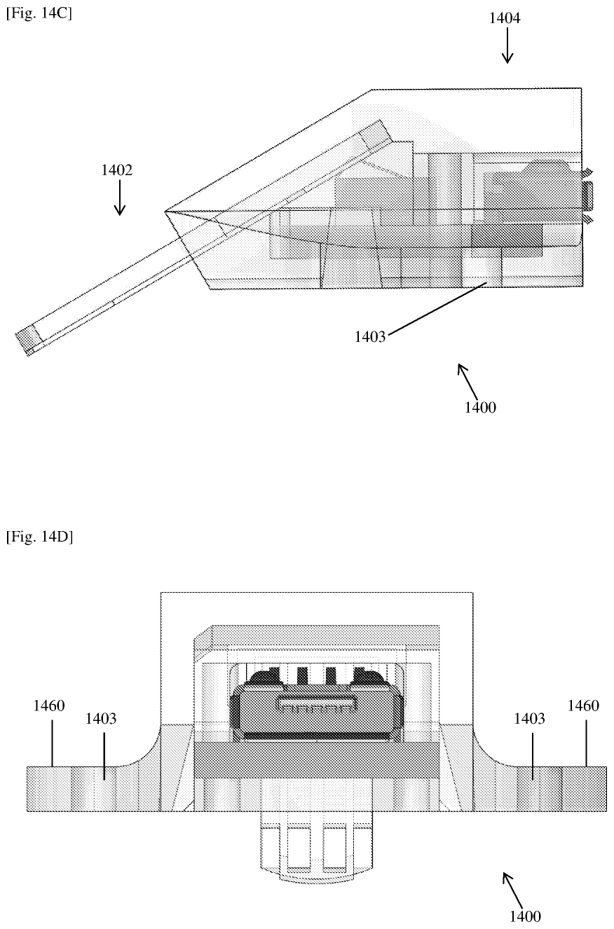

Referring to FIG. 14A, FIG. 14A shows a schematic diagram (zoomed-in isometric view) of a sensor, according to an example embodiment. Similar to FIG. 2, the sensor 1400 includes an elongated body portion 1404 (partially shown in FIG. 14A) and a tip portion 1402. The elongated body portion 1404 includes suturing through-holes 1403 with axes orthogonal to the body axis 604a (as shown in FIG. 6) which allows for deployment of sutures for the securement of the sensor 1400 to a tissue. The elongated body portion 1404 may include flanges 1460 with suturing through-holes 1403. Preferably, the diameter of the suturing through-holes 1403 is at least 0.3 mm Similar to FIG. 2, the sensor 1400 further includes a connector (not shown in FIG. 14A). The connector includes an interface for connection to a reader, a display module, or a monitor device.

Referring to FIG. 14B, FIG. 14B shows a schematic diagram (zoomed-in top view) of a sensor, according to an example embodiment.

Referring to FIG. 14C, FIG. 14C shows a schematic diagram (zoomed-in side view) of a sensor, according to an example embodiment. Similar to FIG. 6, the elongated body portion 1404 has a body axis extending centrally through the elongated body portion 1404. The tip portion 1402 has a tip axis extending centrally through the tip portion 1402. The tip portion 1402 is disposed adjacent the elongated body portion 1404 and at an obtuse angle of between 90° to 170° between the body axis and the tip axis. More preferably, the obtuse angle between the body axis and the tip axis is about 130° to 160°.

Referring to FIG. 14D, FIG. 14D shows a schematic diagram (zoomed-in back view) of a sensor, according to an example embodiment.

Embodiments of the invention may also include one or more bio-compatible adhesive layers disposed at an underside of an elongated body portion and/or tip portion of a sensor, such that the bio-compatible adhesive layers can be in contact with a tissue for the securement of the sensor to the tissue through adhesive means.

It will be envisioned by a person skilled in the art that one or more of the securing mechanisms described above can be used in combination. For example, the inflatable member 312 of sensor 300 as shown in FIG. 3 can be used in combination with the suturing through-holes 1403 of sensor 1400 as shown in FIG. 14A and further used in combination with the bio-compatible adhesive layers.

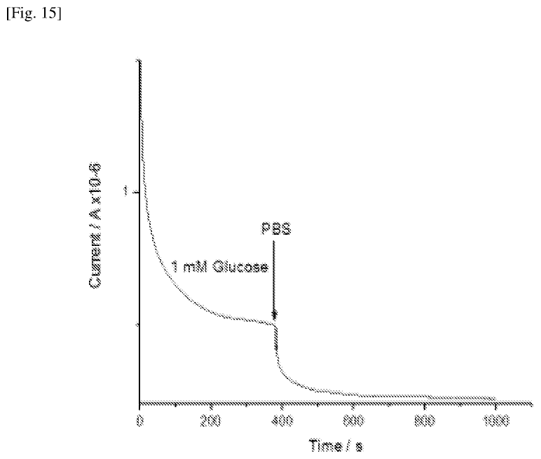

Referring to FIG. 15, FIG. 15 shows a change in the current signal upon the dilution of 1 mM of glucose with a phosphate buffer solution. When the glucose level is reduced, the current signal reading may be different from the original current signal reading. FIG. 15 demonstrates that when the glucose level decreases, the current signal reading may drop accordingly. The glucose oxidase enzyme and the redox polymer were mixed together with the cross-linker and deposited onto the sensor surface. During the test, the sensor was inserted to the glucose solution and an electrochemical reaction occurred between the enzyme and glucose. The electrons released from the reaction were moving between the enzyme-containing polymer and the sensor surface, and were converted to a current signal by a potentiostat. When the glucose concentration decreases upon the dilution with PBS solution, the current signal value also decreased. For the in-vivo applications, when the tissue is failing, the glucose level will decrease and the current signal value monitored by the tissue sensor will also decrease, whereas the current signal value being monitored by the control sensor would be consistent.

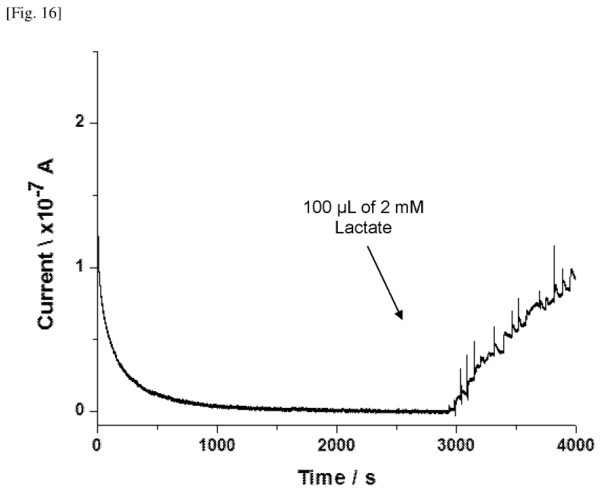

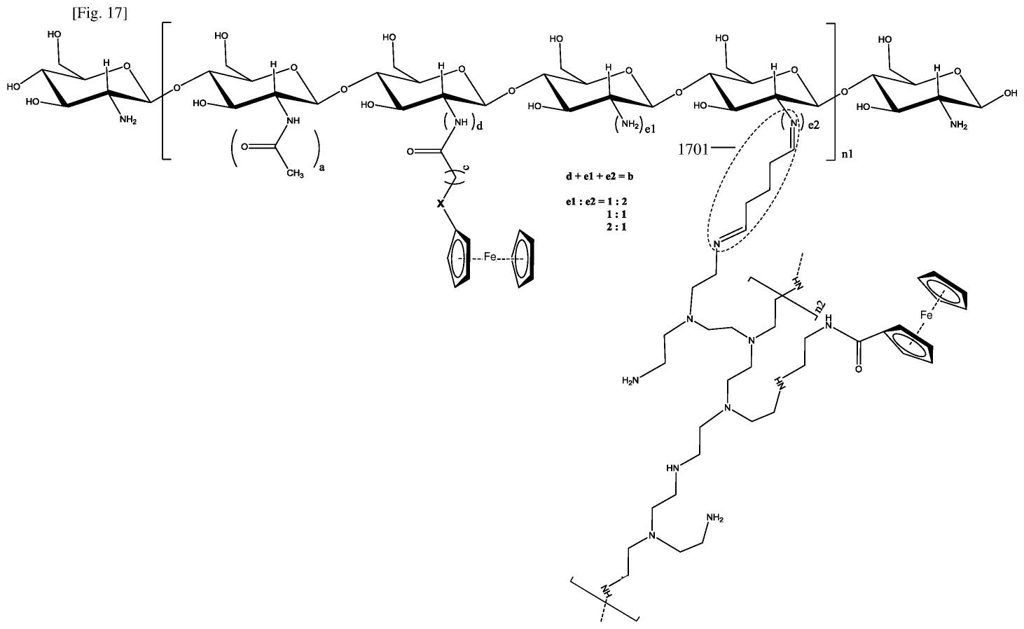

Referring to FIG. 16, FIG. 16 shows a change in the current upon the continuous addition of 100 μL of 2 mM lactate into 10 mL of phosphate buffer solution (PBS) under stirring. When the lactate level increases, the current signal reading may be different from the original current signal reading. FIG. 16 demonstrates that when the lactate level increases, the current signal reading may increase and remains at a certain level. The lactate oxidase enzyme and the redox polymer were mixed together with the cross-linker and deposited onto the sensor surface. During the test, the sensor was inserted to the lactate solution and an electrochemical reaction occurred between the enzyme and lactate. The electrons released from the reaction were moving between the enzyme-containing polymer and the sensor surface, and were converted to a current signal by a potentiostat. When the lactate concentration increases, the current signal value also increased.

For the in-vivo applications, when the tissue is failing, the lactate level will increase and the current signal value monitored by the tissue sensor will also increase, whereas the current signal value being monitored by the control sensor would be consistent.

Referring to FIG. 17, FIG. 17 shows the structure of chitosan-ferrocenyl/branched polyethylenimine-ferrocenyl (CHIT-Fc/BPEI-Fc) conjugate whereby the chitosan-ferrocenyl (CHIT-Fc) redox polymer was cross-linked with branched polyethylenimine-ferrocenyl (BPEI-Fc) intermediate via a cross-linker (1701), glutaraldehyde (GA). The ratio between the deacetylated monomer unit (e1) and the monomer unit crosslinked with the branched polyethylenimine-ferrocenyl intermediate (e2) may be ranging from 1:2, 1:1 and 2:1. The monomer unit having the ferrocenyl derivative (d), the deacetylated monomer unit (e1) and the monomer unit crosslinked with the branched polyethylenimine-ferrocenyl (e2) would summed up to the total deacetylated monomer units of chitosan (b).

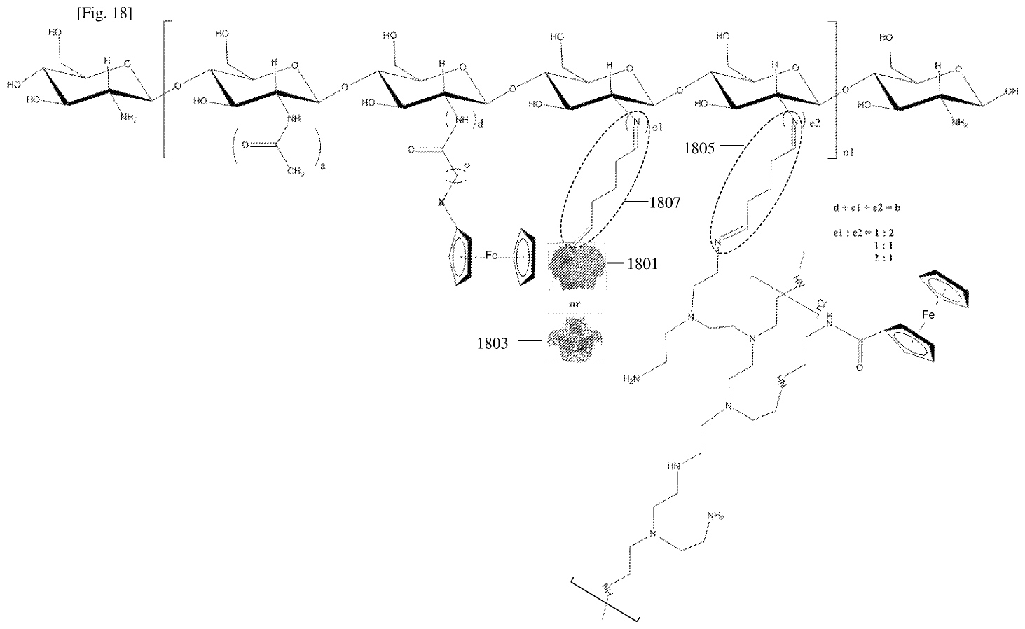

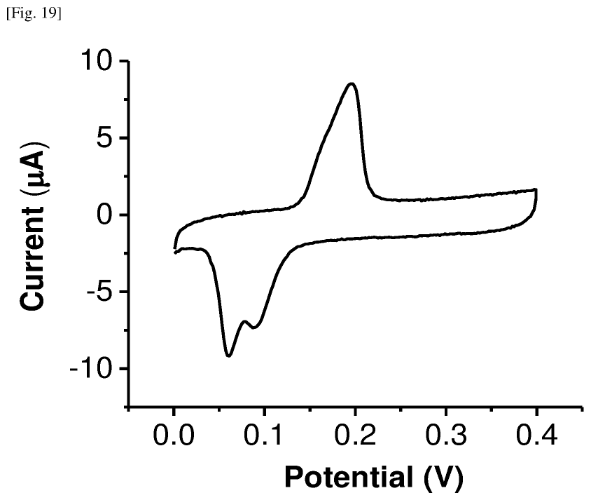

Referring to FIG. 18, FIG. 18 shows the enzyme-containing polymer whereby the enzyme may be a glucose oxidase (1801) or lactate oxidase (1803) and the cross-linkers (1805 and 1807). The ratio between the monomer unit having the enzyme (e1) and the monomer unit crosslinked with the branched polyethylenimine-ferrocenyl intermediate (e2) may be ranging from 1:2, 1:1 and 2:1. The monomer unit having the ferrocenyl derivative (d), the monomer unit having the enzyme (e1) and the monomer unit crosslinked with the branched polyethylenimine-ferrocenyl (e2) would summed up to the total deacetylated monomer units of chitosan (b).

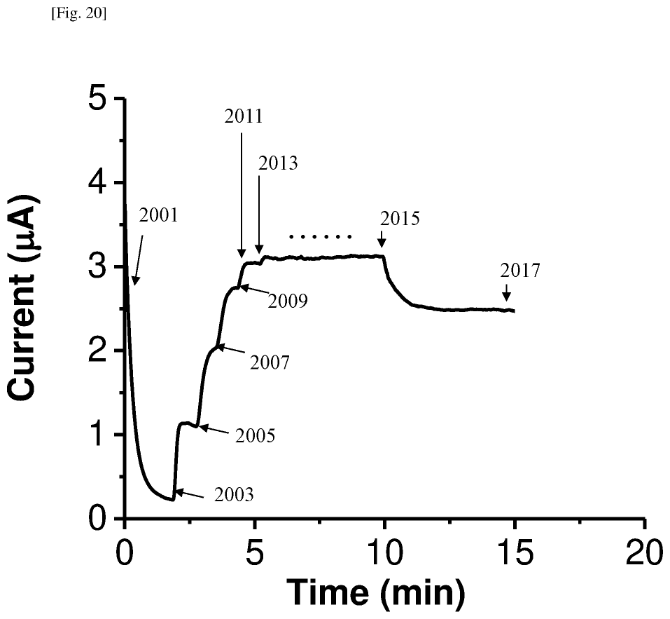

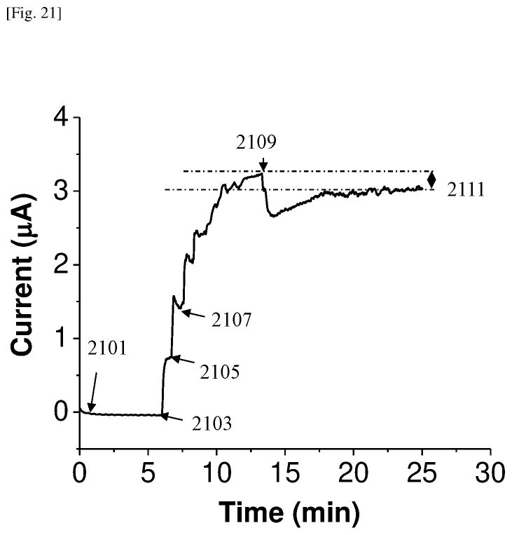

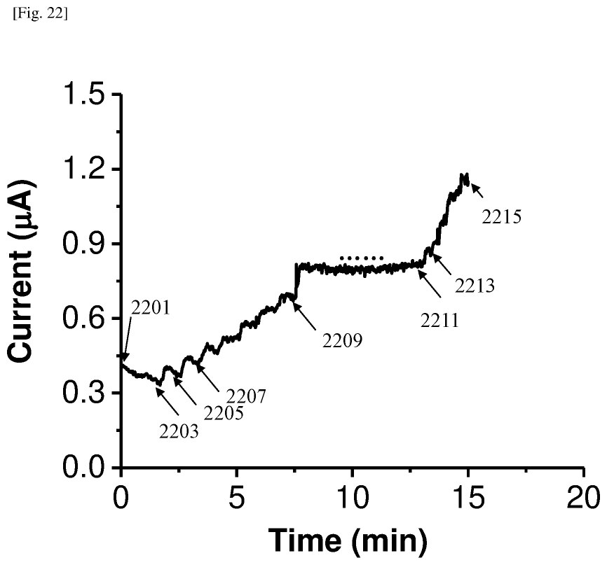

Referring to FIG. 20, FIG. 20 shows an amperometric measurement graph (current over time) to record the current change over time, for the enzyme-containing polymer (glucose oxidase), whereby the sensitivity on glucose was tested. The volume ratio between chitosan-ferrocenyl/branched polyethylenimine-ferrocenyl (CHIT-Fc/BPEI-Fc) conjugate and glucose oxidase is 3:1, whereby the two reactants were crosslinked by the crosslinker. The concentration of glucose oxidase was 20 mg/mL in 1×PBS buffer solution. The current was recorded from 0 seconds (2001), where for the first 120 seconds (2 minutes) the current signal was considered as baseline since no metabolites were in electrolyte solution to react with formulation (redox conjugation and enzyme). After the first 120 seconds (2 minutes), glucose was added into the PBS electrolyte solution, whereby more glucose was added after every 50 seconds or 100 seconds. This addition allowed the amount of glucose to increase to 0.5 mM (2003) and 1.0 mM (2005). As the amount of glucose increased to 1.5 mM (2007), 2.0 mM (2009) and 2.5 mM (2011), the current also increased with time until the amount of glucose was 3.0 mM (2013) that the graph or the current flattens to a constant value of about 3.1 μA. About 5 minutes later, in order to mimic the flap failure, 5 mL of PBS solution was added (2015), whereby the concentration of glucose decreased to 1.8 mM (2017) and that was the final concentration of glucose. When the concentration of glucose gradually declined, the current decreased over time as well.

Referring to FIG. 21, FIG. 21 shows an amperometric measurement graph (current over time) to record the current change over time, for the enzyme-containing polymer (glucose oxidase), whereby the sensitivity on glucose was tested. The volume ratio between chitosan-ferrocenyl/branched polyethylenimine-ferrocenyl (CHIT-Fc/BPEI-Fc) conjugate and glucose oxidase was 3:2, whereby the two reactants were crosslinked by the crosslinker. The concentration of glucose oxidase was 20 mg/mL in 1×PBS buffer solution. The current was recorded from 0 seconds (2101), where for the first 120 seconds (2 minutes) the current signal was considered as baseline since no metabolites were in electrolyte solution to react with formulation (redox conjugation and enzyme). After the first 300 seconds (5 minutes), glucose was added into the PBS electrolyte solution, whereby more glucose was added after every 50 seconds or 100 seconds. This addition allowed the amount of glucose to increase to 0.2775 mM (2103) and 0.555 mM (2105). As the amount of glucose increased to 0.8325 mM (2107), the current also increased with time until PBS solution was added at 2109 to reduce/dilute the concentration of glucose. This is to mimic the flap failure, and indeed the current decreased due to the reduced concentration of glucose or the dilutions (2111).

Referring to FIG. 22, FIG. 22 shows an amperometric measurement graph (current over time) to record the current change over time, for the enzyme-containing polymer (lactate oxidase), whereby the sensitivity on lactate was tested. The volume ratio between chitosan-ferrocenyl/branched polyethylenimine-ferrocenyl (CHIT-Fc/BPEI-Fc) conjugate and lactate oxidase was 3:1, whereby the two reactants were crosslinked by the crosslinker. The concentration of lactate oxidase was 20 mg/mL in 1×PBS buffer solution. The current was recorded from 0 seconds (2201), where for the first 120 seconds (2 minutes) the current signal was considered as baseline since no metabolites were in electrolyte solution to react with formulation (redox conjugation and enzyme). After the first 120 seconds (2 minutes), sodium lactate was added into the PBS electrolyte solution, whereby more sodium lactate was added after every 50 seconds or 100 seconds. This addition allowed the amount of lactate to increase to 0.024 mM (2203) and 0.048 mM (2205). As the amount of lactate increased to 0.072 mM (2207) and 0.192 mM (2209), the current also increased with time until the graph or the current flattens to a constant value of about 0.8 μA. About 5 minutes later, in order to mimic the flap failure, more sodium lactate was added at 2211 and the concentration of lactate increased to 0.216 mM (2213) and increased till the final amount of approximately 0.336 mM (2215). When the concentration of sodium lactate gradually increased, the current increased over time as well.

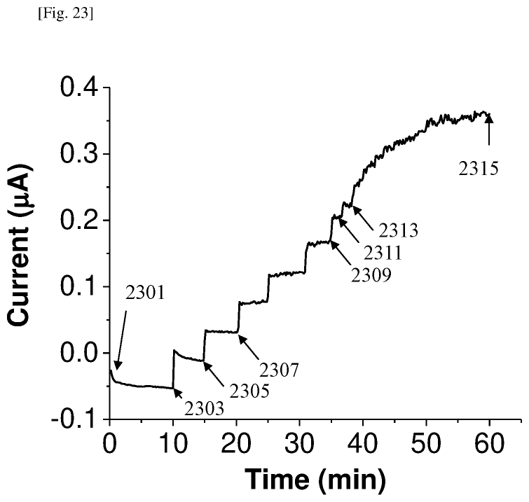

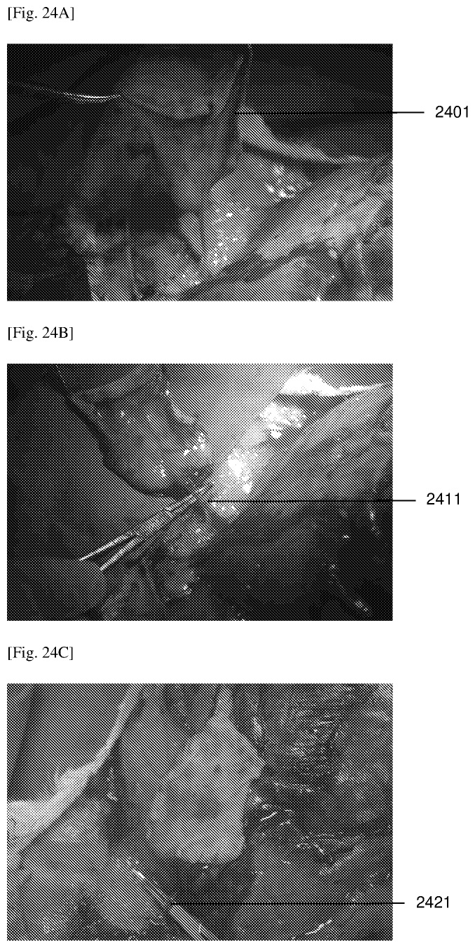

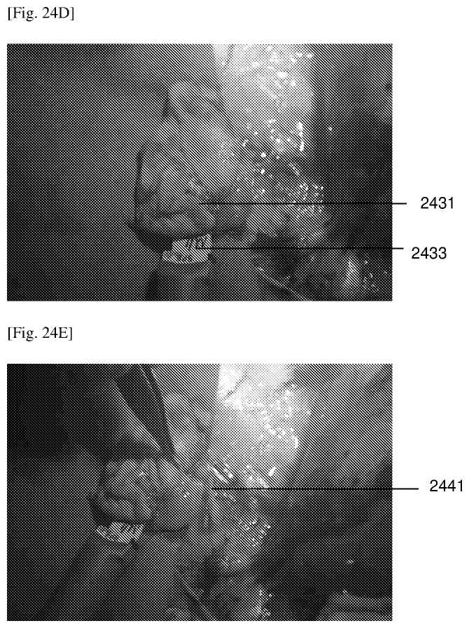

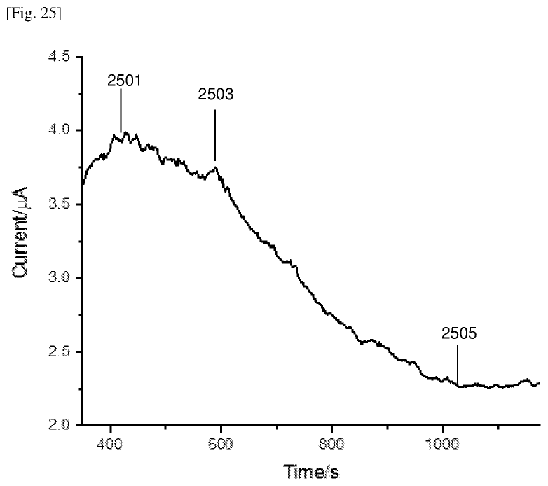

Referring to FIG. 23, FIG. 23 shows an amperometric measurement graph (current over time) to record the current change over time, for the enzyme-containing polymer (lactate oxidase), whereby the sensitivity on lactate was tested. The volume ratio between chitosan-ferrocenyl/branched polyethylenimine-ferrocenyl (CHIT-Fc/BPEI-Fc) conjugate and lactate oxidase was 3:2, whereby the two reactants were crosslinked by the crosslinker. The concentration of lactate oxidase was 20 mg/mL in 1×PBS buffer solution. The current was recorded from 0 seconds (2301), where for the first 120 seconds (2 minutes) the current signal was considered as baseline since no metabolites were in electrolyte to react with formulation (redox conjugation and enzyme). After the first 600 seconds (5 minutes), sodium lactate was added into the PBS electrolyte solution, whereby more sodium lactate was added after every 50 seconds or 100 seconds. This addition allowed the amount of lactate to increase to 0.0892 mM (2303) and 0.1784 mM (2305). As the amount of lactate increased to 0.2676 mM (2307), 0.5354 mM (2309), 0.58 mM (2311) and 0.6246 mM (2313), the current also increased with time. In order to mimic the flap failure, more sodium lactate was added at 2313 and about 20 minutes later, the concentration of lactate increased to a final concentration of 0.94 mM (2315). When the concentration of sodium lactate gradually increased, the current increased over time as well.

Referring to FIG. 24, FIG. 24A shows the flap (2401) with a skin paddle of 3 cm by 5 cm being raised from the rabbit. FIG. 24B and FIG. 24C show that the skin incisions were made and the flap was islanded based on the inferior epigastric blood vessel (2411 and 2421) supplying to the muscle and skin. Bilateral flaps were designed based on blood supply from the inferior epigastric vessels (2411 and 2421) on each side of the rabbit. FIG. 24D shows the sensor chip (2433) coated with the enzyme-containing polymer being wrapped around with the flap (2431). FIG. 24E shows the blood vessel being clamped (2441) for the glucose and lactate tests.

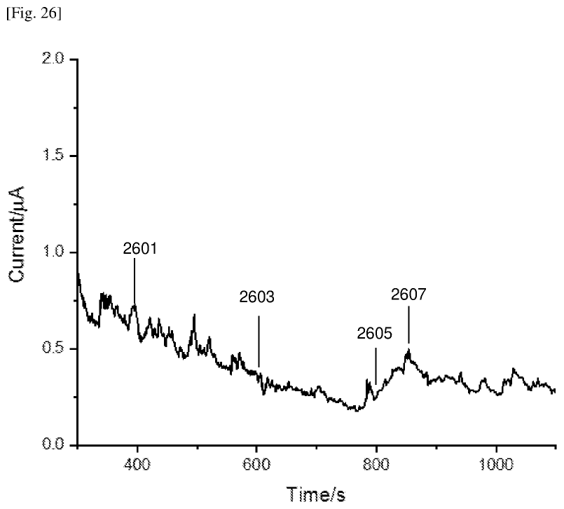

Referring to FIG. 25, FIG. 25 shows an amperometric measurement graph (current over time) to record the current change over time for glucose (2501), before and after clamping (2503) of the blood vessel on the flap, at 600 seconds. There was a significant drop in current from about 3.75 μA at 600 seconds (2503) to about 2.25 μA at 1100 seconds (2505).

Referring to FIG. 26, FIG. 26 shows an amperometric measurement graph (current over time) to record the current change over time for lactate (2601), before and after clamping (2603) of the blood vessel on the flap, at 600 seconds. At about 800 seconds, the current began to increase from about 0.20 μA (2605) to about 0.50 μA at 850 seconds (2607).

### EXAMPLES

Non-limiting examples of the disclosure will be further described in greater detail by reference to specific Examples, which should not be construed as in any way limiting the scope of the invention.

### List of Abbreviations Used

Ace: acetone

AcOH: acetic acid

BPEI: branched polyethylenimine

CHCl₃: chloroform

CHIT: chitosan

DI: deionized water

EDC.HCl: N-Ethyl-N′-(3-dimethylaminopropyl)carbodiimide hydrochloride

Ether: diethylether

Et₃N: triethylamine

EtOAc: ethyl acetate

EtOH: ethanol

FcCOOH: ferrocenecarboxylic acid

FcCOCl: chlorocarbonyl ferrocene or ferrocenoyl chloride

GA: glutaraldehyde

h: hour(s)

HCl: hydrochloric acid

IPA: iso-propanol (2-propanol)

L: litre(s)

Me: methyl

MeOH: anhydrous methanol

min: minute(s)

NaOH: sodium hydroxide

NHS: N-hydroxysuccinimide

K₂CO₃: potassium carbonate

PBS: phosphate-buffered saline

Sec: seconds

Materials and Methods

Chitosan flakes (from shrimp shells, minimum 75% deacetylated), glucose oxidase (EC 1.1.3.4, lyophilized powder, 200 U/mg) and lactate oxidase (from Aerococcus viridans) were purchased from Sigma-Aldrich (St. Louis, Mo., United States). Branched polyethyleneimine, glucose, sodium lactate and glutaraldehyde were obtained from Sigma-Aldrich. (6-Bromohexyl) ferrocene, ferrocenecarboxylic acid, chlorocarbonyl ferrocene and other ferrocenyl derivatives were obtained from PICHEM (Shanghai, China). Other reagents including acetic acid, hydrochloric acid, potassium carbonate, sodium hydroxide, N-hydroxysuccinimide, triethylamine and 1 N-ethyl-N′-(3-dimethylaminopropyl)carbodiimide hydrochloride were also obtained from Sigma-Aldrich. Solvents including, chloroform, ethyl acetate, acetone, hexane, methanol, ethanol, isopropanol, propanol and PBS buffer solution were obtained from Sigma-Aldrich. All reagents and solvents were ACS reagent grade and were used as received unless noted otherwise. The PBS (1×) buffer solution was diluted from the 10×PBS stock solution, and the ready-to-use buffer solutions were kept in a 4° C. fridge to avoid bacterial contamination. When required, 5 to 20 mL of PBS (1×) buffer solution was taken from the main stock and transferred to sample vial for use. Stirring was applied during the glucose and lactate analysis.

Sensitivity Test on Metabolites (Glucose and Lactate)

Sensor Chip Preparation

The sensor chip was pre-cleaned by rinsing with deionized (DI) water and ethanol (EtOH), and dried with nitrogen flow. The precursor of the enzyme-containing polymer together with the respective enzyme (conjugate CHIT-Fc/BPEI-Fc:GOx and CHIT-Fc/BPEI-Fc:LOx) were prepared in condensed solution. The volume ratio between chitosan-ferrocenyl/branched polyethylenimine-ferrocenyl (CHIT-Fc/BPEI-Fc) conjugate and glucose oxidase or lactate oxidase was 3:1, whereby both reactants were crosslinked by the crosslinker. The concentration of glucose oxidase or lactate oxidase was 20 mg/mL in 1×PBS buffer solution. The volume ratio between chitosan-ferrocenyl/branched polyethylenimine-ferrocenyl (CHIT-Fc/BPEI-Fc) conjugate and glucose oxidase or lactate oxidase was also prepared at 3:2, whereby the two reactants were crosslinked with the same crosslinker. The concentration of glucose oxidase or lactate oxidase was also 20 mg/mL in 1×PBS buffer solution. The formulation solution was carefully dropped on the working electrode of DropSens chip (i.e., round disk in center) and left to dry overnight at room temperature to form a thin layer of film on the electrode.

Testing Set-Up

The sensor chip connector cable was fixed by an iron support while the other end was connected with a potentiostat. The sensor chip (electrode pads end) was then fitted in with the connector cable and the working electrode end was immersed in the electrolyte solution.

Time control and voltage control in i-t scan, amperometry 0.4 V-0.6 V of voltage was applied to activate the reaction between redox conjugate and enzyme to produce current signals. The test period was set from 15 minutes to 2 hour on-demand

Materials and Methods for Animal Studies

For the animal studies, the species selected is rabbit and the stock is from New Zealand White, where the weight is 3 kg and the gender of the rabbit is female. The rabbit is housed in the animal facility at SingHealth Experimental Medicine Centre, Singapore. All surgeries were performed under general anaesthesia. All surgeries were performed under aseptic conditions using sterile surgical instruments. Induction antibiotics and analgesics: Enrofloxacin (5 mg/kg subcutaneously) and Carprofen (5 mg/kg BW) were given. Electrical warming pads with a temperature probe were used for all surgical procedures. Bilateral rectus abdominis musculocutaneous flap with a skin paddle of size 3 cm by 5 cm was raised from the rabbit. Skin incisions were made and the flap was islanded based on the inferior epigastric blood vessels supplying to the muscle and skin. The bilateral flaps were designed based on the blood supply from the inferior epigastric vessels on each side of the rabbit. This flap that was raised is a dermal (skin) flap that comprised fats and skin tissues, as opposed to being a skeletal muscular (muscle) flap that is comprised of mainly of muscle tissue. In this case, the skin flap would show a less pronounced rise in lactate levels after the blood supply is stopped, than a muscle flap.

### Example 1

Synthesis of Hexylferrocenyl-Chitosan Redox Polymer

A 1.0% chitosan (Chi) solution is prepared by dissolving 1.0 g of chitosan flakes into 100 mL of 1.0% acetic acid and stirred for 3 hours at room temperature until complete dissolution. The solution is stored in refrigerator when not in use. Briefly, 1 mg of Chi (≥75% deacetylated) was added into 20 mL of isopropanol 4N sodium hydroxide solution, and the mixture was heated to reflux. (6-bromohexyl) ferrocene was slowly added into the polymer solution using a pipette. The mixture was heated to reflux under nitrogen for 4 hours; the solvent was removed under reduced pressure. The residue was repetitively washed with diethyl ether (×3) and methanol (×2) to remove residual impurities and then dried under vacuum. The resultant hexylferrocenyl chitosan was then dialyzed for 3 days against DI water.

Preparation of Redox Polymer-Enzyme Sensors

Redox Polymer-Glucose Oxidase Enzyme

The synthesized redox polymer was dissolved into HOAc/OAc buffer solution (pH 5) until the final concentration of polymer solution was 10 mg/mL. 10 μL of 10 mg/mL redox polymer solution, 5 μL of 10 mg/mL glucose oxidase (GOx) and 1 μL of 1% glutaraldehyde (GA) as cross-linker were mixed together and place onto the sensor surface. The mixture was then allowed to cure for 12 hours. Finally, another layer of biofilm Nafion was coated onto the sensor surface as the protective layer.

Redox Polymer-Lactate Oxidase Enzyme

The synthesized redox polymer was dissolved into HOAc/OAc buffer solution (pH 5) until the final concentration of polymer solution was 10 mg/mL. 10 μL of 10 mg/mL redox polymer solution, 5 μL of 10 mg/mL lactate oxidase (LOx) solution, and 1 μL of 1% glutaraldehyde (GA) as cross-linker were mixed together and place onto the sensor surface. The mixture was then allowed to cure for 12 hours. Finally, another layer of biofilm Nafion was coated onto the sensor surface as the protective layer.

The redox polymer has the structure below:

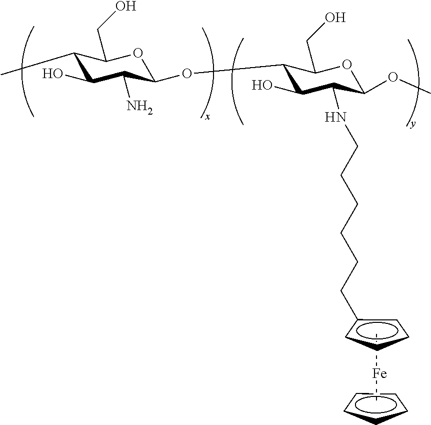

The enzyme-containing polymer has the structures below:

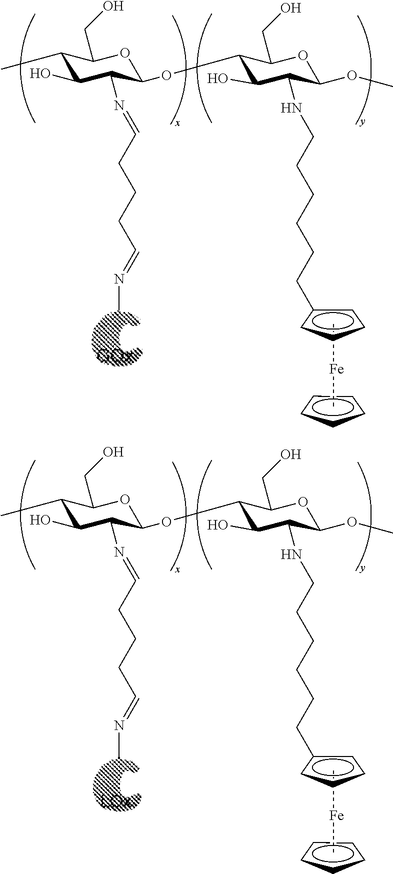

### Example 2

Step One: Synthesis of Chitosan-Ferrocenyl (CHIT-Fe) Redox Polymer (Crude Product)

FcCOOH was attached to CHIT with 40% w/w grafting ratio: CHIT.HCl was dissolved in DI water, and FcCOOH was dissolved in anhydrous MeOH and activated by EDC.HCl and NHS for 30 minutes. FcCOOH/EDC HCl/NHS mixture was then added into CHIT.HCl solution dropwise. The reaction was stirred for 12 hours under inert nitrogen gas. The CHIT-Fc redox polymer was obtained by neutralizing with 0.1 M NaOH solution (pH was adjusted to pH 10), then washed with DI water and MeOH several times. The precipitation obtained was dried and then dissolved in 1 wt % acetic acid solution (5 mg/ml), as shown in Scheme 1.

Step Two: Synthesis of Branched Polyethylenimine-Ferrocenyl (BPEI-Fc) Intermediate (Crude Product)

FcCOCl was attached to BPEI with 40% w/w grafting ratio: BPEI and FcCOCl were dissolved in anhydrous MeOH in 5% w/v, separately. FcCOCl was added into BPEI solution dropwise and few drops of Et₃N were added into the reaction mixture. The reaction was stirred for 12 hours under inert nitrogen gas. BPEI-Fc intermediate was obtained by precipitating in CHCl₃/Hexane (1:4 in v/v) mixed solvent. The precipitation obtained was washed with MeOH and re-precipitated twice. The re-precipitated precipitation was dried and then dissolved in 1×PBS (35 mg/ml), as shown in Scheme 2.

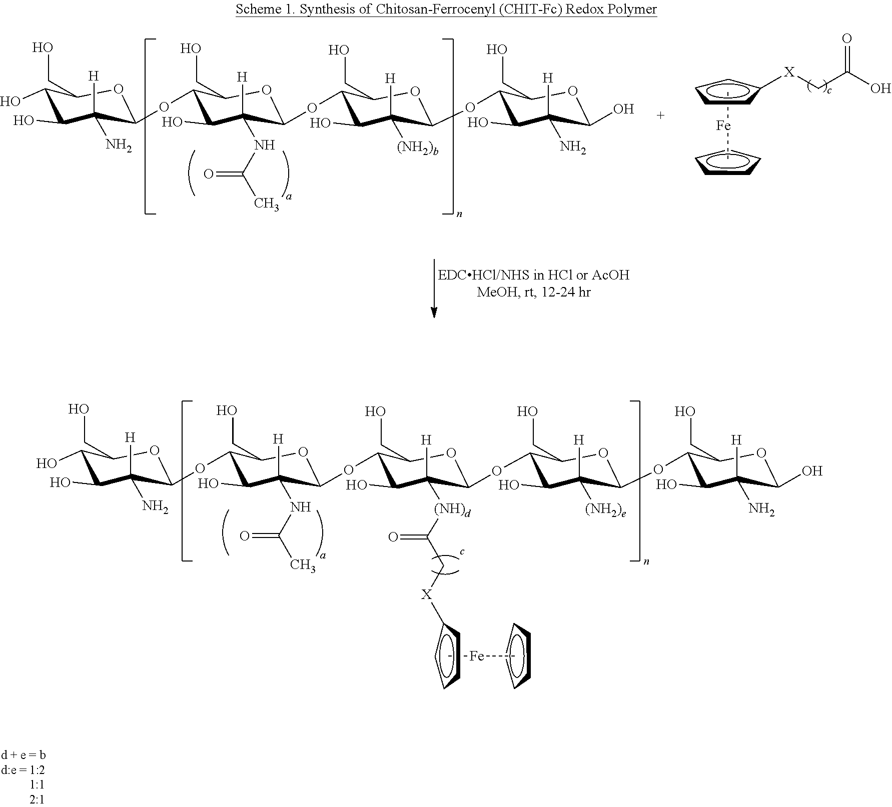

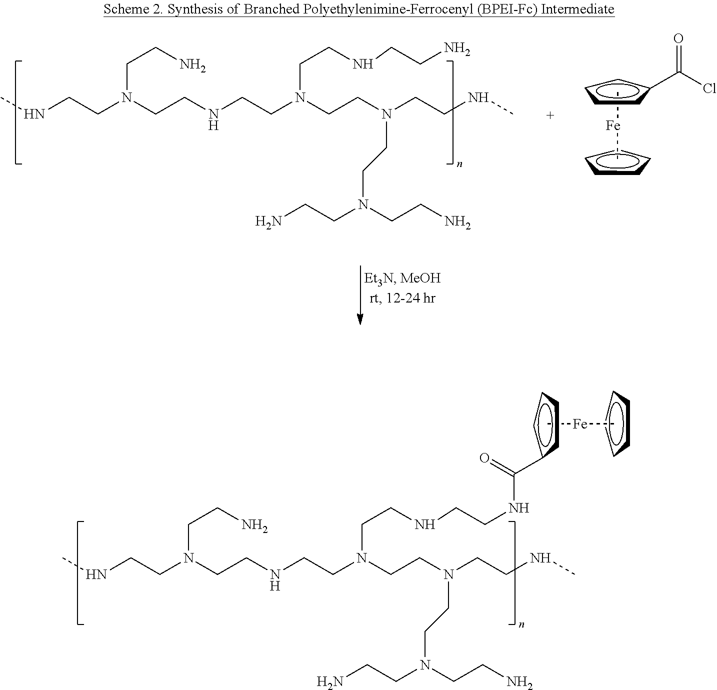

Step Three: Synthesis of Chitosan-Ferrocenyl/Branched Polyethylenimine-Ferrocenyl (CHIT-Fc/BPEI-Fc) Conjugate and the Enzyme-Containing Polymer

16 uL of BPEI-Fc (dissolved in 1×PBS (35 mg/mL)) was fully mixed and stirred with 120 uL of CHIT-Fc (dissolved in 1 wt % acetic acid (5 mg/mL)) in the presence of 8 uL of GA solution (25 wt % in water diluted in 1×PBS in 5 mg/mL) for 2 hours at room temperature to yield the CHIT-Fc/BPEI-Fc conjugate as shown in FIG. 17.

The CHIT-Fc/BPEI-Fc conjugate was then divided into two portions and mixed with the respective enzymes (glucose oxidase and lactate oxidase) to yield the enzyme-containing polymer as indicated in FIG. 18.

### Example 3

Sensitivity Test on Metabolites (Glucose and Sodium Lactate)

The sensitivity test was performed once the sensor chip was coated with a thin layer of the enzyme-containing polymer (conjugate and the respective enzyme) and dipped into the PBS electrolyte solution. The thin layer of enzyme-containing polymer was prepared as mentioned above. The PBS electrolyte solution was also prepared based on the methods as mentioned above.

For the glucose testing, two different volume ratios of chitosan-ferrocenyl/branched polyethylenimine-ferrocenyl (CHIT-Fc/BPEI-Fc) conjugate and glucose oxidase were prepared. For the first volume ratio of 3:1, the current was measured and recorded when time is 0 seconds, where the first 120 seconds (2 minutes) is considered as baseline since no metabolites were present in the PBS electrolyte solution to react with the thin film of enzyme-containing polymer. At 120 seconds, glucose was added into the PBS electrolyte solution, followed by every 50 or 100 seconds to increase the glucose concentration. More glucose was added to the electrolyte solution until the current flattens to a constant value and to mimic flap failure, the concentration of glucose was reduced by diluting with PBS electrolyte solution (2015), as indicated in FIG. 20. The current decreases as the concentration of glucose decreases to 1.8 mM (2017). When the volume ratio between chitosan-ferrocenyl/branched polyethylenimine-ferrocenyl (CHIT-Fc/BPEI-Fc) conjugate and glucose oxidase was changed to 3:2, the polymer was also coated on the chip and used for testing as indicated in FIG. 21. Likewise, the current for the first 120 seconds or more is considered as baseline. After the first 300 seconds (5 minutes), when glucose was added to the electrolyte solution, the current increased as the concentration of glucose increased until 2109 of FIG. 21, where dilution occurred after more PBS electrolyte solution was added. Once the concentration of glucose decreased, the current also decreased as indicated at 2111 of FIG. 21. The detecting sensitivity on glucose was as low as 0.28 mM per change for this formulation.

For the lactate testing, two different volume ratios of chitosan-ferrocenyl/branched polyethylenimine-ferrocenyl (CHIT-Fc/BPEI-Fc) conjugate and lactate oxidase were prepared. For the first volume ratio of 3:1, the current was measured and recorded when time is 0 seconds, where the first 120 seconds (2 minutes) is considered as baseline since no metabolites were present in the PBS electrolyte solution to react with the thin film of enzyme-containing polymer. At 120 seconds, lactate was added into the PBS electrolyte solution, followed by every 50 or 100 seconds to increase the lactate concentration. More lactate was added to the electrolyte solution until the current flattens to a constant value and to mimic flap failure, the concentration of lactate was further increased by adding more lactate to the PBS electrolyte solution at 2211 of FIG. 22. The current continued to increase as the lactate increased to 0.336 mM (2215). When the volume ratio between chitosan-ferrocenyl/branched polyethylenimine-ferrocenyl (CHIT-Fc/BPEI-Fc) conjugate and lactate oxidase was changed to 3:2, the polymer was also coated on the chip and used for testing as indicated in FIG. 23. Likewise, the current for the first 120 seconds is considered as baseline. After about 600 seconds (5 minutes), when lactate was added to the electrolyte solution, the current increased as the concentration of lactate increased until the final concentration of 0.94 mM (2315) of FIG. 23. The detecting sensitivity on sodium lactate was as low as 0.024 mM per change for this formulation.

### Example 4

Animal Studies Glucose and Lactate Measurements Using the Sensor Chip Coated with the Enzyme-Containing Polymer

Using the rabbit in the animal study, the flap (2401) was prepared based on the method as mentioned above and as indicated in FIG. 24A to FIG. 24E. To measure the glucose and lactate levels on the flap, a vessel occlusion was mimicked by cutting off the blood supply (2411 and 2421) of the flap, by clamping (2441) the area surrounding the sensor chip. The sensor chip was coated with a thin layer of the enzyme-containing polymer and was wrapped around with the flap (2433).

For the glucose sensor chip test, a standard electrochemical method, amperometry was used to record the current change after the clamp was fastened on the flap. As shown in FIG. 25, in the current-time (i-t) scan for the glucose analysis (2501), the current was recorded from 0 seconds, followed by clamping of the blood vessel being performed at 600 seconds (2503). A significant drop in current was observed almost immediately after the blood vessel in the flap was being clamped. The current decreased from about 3.75 μA at 600 seconds to about 2.25 μA at 1100 seconds (2505). This result indicated that once the blood supply is stopped, the amount of glucose present in the specimen would be reduced.

For the lactate sensor chip test, a standard electrochemical method, amperometry was used to record the current change after the clamp was fastened on the flap. As shown in FIG. 26, in the current-time (i-t) scan for the lactate analysis (2601), the current was recorded from 0 seconds, followed by clamping of the blood vessel being performed at 600 seconds (2603). An increase in current was observed after the blood vessel in the flap was being clamped, at about 800 seconds. The current increased from about 0.18 μA at 800 seconds to about 0.50 μA at 850 seconds. The delay in the current response was due to the slow accumulation of lactate in the flap after the vessel was clamped. Since this flap is a skin flap, comprising mainly fats and skin tissues, the rise in lactate level is less pronounced (i.e. there is an increment of current only at 800 seconds).

### INDUSTRIAL APPLICABILITY

The disclosed enzyme-containing polymer may be used to detect the presence of a metabolite in a tissue. The disclosed enzyme-containing polymer may be used in a sensor to detect the presence of a metabolite in a tissue. Where two or more different enzyme-containing polymers are used in the sensor, the amounts of two or more metabolites can be measured, and the relationship between the amounts of the metabolites may signal whether the tissue is healthy or may fail. Thus, the enzyme-containing polymer as well as the associated sensor (or device) may be used in a clinical setting to facilitate monitoring of tissue by doctors and/or nurses. The sensor or device may be used even when the tissue is buried.

It will be apparent that various other modifications and adaptations of the invention will be apparent to the person skilled in the art after reading the foregoing disclosure without departing from the spirit and scope of the invention and it is intended that all such modifications and adaptations come within the scope of the appended claims.

1. An enzyme-containing polymer comprising:
a first repeating unit of Formula Ia: 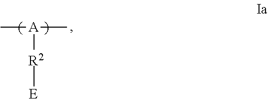
a second repeating unit of Formula Ib: 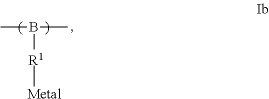
and
a third repeating unit of Formula Ic: 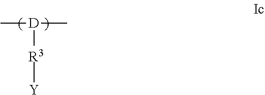
or conjugate salts thereof, wherein
each of A, B, and D is independently a 2-amino monosaccharide;
E is an enzyme comprising an n-terminal amine and optionally one or more lysine residues, wherein R² is covalently bonded to the n-terminal amine or the amine side chain of the one or more lysine residues;
Metal is a metal complex having a redox potential lower than hydrogen peroxide under physiological conditions;
R¹ is —N*(R)(C═O)—, —N*(R)(C═O)N(R)—, —N*(R)(CR₂)ₙ—, —N*(R)(CR₂)ₙ(C═O)—, —N*(R)(C═O)(CR₂)ₙ—, —N*(R)(C═O)(CR₂)ₙ(C═O)—, —N*(R)(CR₂)ₙO(CR₂)ₘ—, —N*(R)(CR₂)ₙS(CR₂)ₘ—, —N*(R)(CR₂)ₙO—, —N*(R)(CR₂)ₙ(C═O)O—, —N*(R)(C═O)(CR₂)ₙO—, —N*(R)(C═O)(CR₂)ₙ(C═O)O—, —N*(R)(CR₂)ₙO(CR₂)ₘO—, —N*(R)(CR₂)ₙS(CR₂)ₘO—, —N*═CH(CR₂)ₙ—, —N*═CH(CR₂)ₙ(C═O)—, —N*═CH(CR₂)ₙO(CR₂)ₘ—, —N*═CH(CR₂)ₙS(CR₂)ₘ—, —N*═CH(CR₂)ₙO—, —N*═CH(CR₂)ₙ(C═O)O—, —N*═CH(CR₂)ₙO(CR₂)ₘO—, or —N*═CH(CR₂)ₙS(CR₂)ₘO—, wherein N* represents the nitrogen at the 2 position of the 2-amino monosaccharide;
R² is —N*(R)(CR₂)ₙN**(R)—, —N*(R)(CR₂)ₙ(C═O)N**(R)—, —N*(R)(C═O)(CR₂)ₙ—, —N*(R)(C═O)(CR₂)ₙ(C═O)N**(R)—, —N*(R)(CR₂)ₙO(CR₂)ₘN**(R)—, —N*(R)(CR₂)ₙS(CR₂)ₘN**(R)—, —N*═CH(CR₂)ₙN**(H)—, —N*═CH(CR₂)ₙ(C═O)N**(R)—, —N*═CH(CR₂)ₙO(CR₂)ₘN**(R)—, —N*═CH(CR₂)ₙS(CR₂)ₘN**(R)—, N*(R)(CR₂)ₙCH═N**—, —N*(R)(C═O)(CR₂)ₙCH═N**—, —N*(R)(CR₂)ₙO(CR₂)ₘCH═N**—, —N*(R)(CR₂)ₙS(CR₂)ₘCH═N**—, —N*═CH(CR₂)ₙCH═N**—, —N*═CH(CR₂)ₙO(CR₂)ₘCH═N**—, or —N*═CH(CR₂)ₙS(CR₂)ₘCH═N**—, wherein N* represents the nitrogen at the 2 position of the 2-amino monosaccharide and N** represents the nitrogen of the n-terminal amine or the amine side chain of the one or more lysine residues of the enzyme; or R² is represented by the moiety: 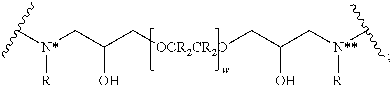
R³ is —N*(R)(CR₂)ₙY, —N*(R)(CR₂)ₙ(C═O)Y, —N*(R)(C═O)(CR₂)ₙY, —N*(R)(C═O)(CR₂)ₙ(C═O)Y, —N*(R)(CR₂)ₙO(CH₂)ₘY, —N*(R)(CR₂)ₙS(CR₂)ₘY, —N*═CH(CR₂)ₙY, —N*═CH(CR₂)ₙ(C═O)Y, —N*═CH(CR₂)ₙO(CR₂)ₘY, —N*═CH(CR₂)ₙS(CR₂)ₘY, —N*(R)(CR₂)ₙCH═Y, —N*(R)(C═O)(CR₂)ₙCH═Y, —N*(R)(CR₂)ₙO(CH₂)ₘCH═Y, —N*(R)(CR₂)ₙS(CR₂)ₘCH═Y, —N*═CH(CR₂)ₙCH═Y, —N*═CH(CR₂)ₙO(CR₂)ₘCH═Y, or —N*═CH(CR₂)ₙS(CR₂)ₘCH═Y, wherein N* represents the nitrogen at the 2 position of the 2-amino monosaccharide; or R³ is represented by the moiety: 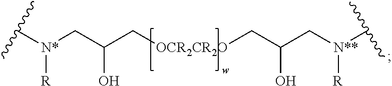
R for each occurrence is independently hydrogen, lower alkyl or hydroxyl;
m for each occurrence is independently a whole number selected between 1-20;
n for each occurrence is independently a whole number selected between 1-20;
w for each occurrence is independently a whole number selected between 1-20; and
Y is a polyalkylamine comprising at least one metal complex, wherein the polyalkylamine optionally crosslinks at least two of the third repeating units.

2. The enzyme-containing polymer of claim 1, wherein the metal complex is optionally substituted ferrocenyl.

3. The enzyme-containing polymer of claim 1, wherein the molar ratio of the first repeating unit to the third repeating unit in the enzyme-containing polymer is between 1:2 to 2:1.

4. The enzyme-containing polymer of claim 1, wherein the 2-amino monosaccharide is selected from the group consisting of glucosamine, mannosamine, and galactosamine.

5. The enzyme-containing polymer of claim 2, wherein the first repeating unit has the Formula IIIa: 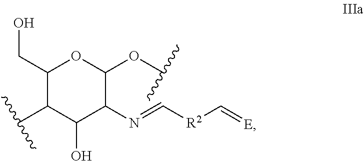
the second repeating unit has the Formula IIIb: 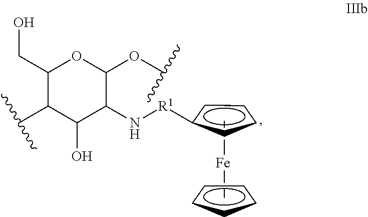
and
the third repeating unit has the Formula IIIc: 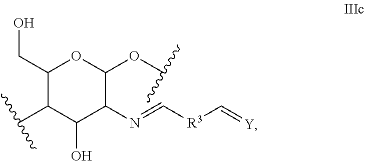
wherein
R¹ is —(C═O)—, —(C═O)(CH₂)ₙ—, —(C═O)(CH₂)ₙ(C═O)—, —(CH₂)ₙ(C═O)O—, or —(C═O)(CH₂)ₙO—;
R² is —(CH₂)ₙ—;
R³ is —(CH₂)ₙ—; and
Y is a branched polyethylenimine comprising at least one primary or secondary amine covalently bonded to a moiety having the structure:

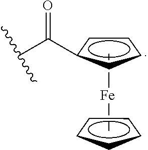

6. The enzyme-containing polymer of claim 5, wherein R¹ is —(C═O)—; R² is —(CH₂)₃—; and R³ is —(CH₂)₃—.

7. The enzyme-containing polymer of claim 5 further comprising a fourth repeating unit of Formula IIId:

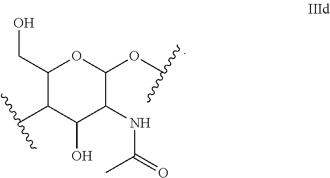

8. The enzyme-containing polymer of claim 5, wherein the enzyme is selected from the group consisting of glucose oxidase, lactate oxidase, xanthine oxidase, cholesterol oxidase, malate oxidase, galactose oxidase, glucose dehydrogenase, lactate dehydrogenase, xanthine dehydrogenase, alcohol oxidase, choline oxidase, xanthine oxidase, glutamate oxidase and amine oxidase.

9. A redox polymer comprising a first repeating unit of Formula IIa: 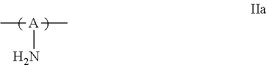
and a second repeating unit of Formula IIb: 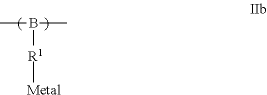
or conjugate salts thereof, wherein
each of A and B independently a 2-amino monosaccharide;
Metal is a metal complex having a redox potential lower than hydrogen peroxide under physiological conditions;
R¹ is —N*(R)(C═O)—, —N*(R)(C═O)N(R)—, —N*(R)(CR₂)ₙ—, —N*(R)(CR₂)ₙ(C═O)—, —N*(R)(C═O)(CR₂)ₙ—, —N*(R)(C═O)(CR₂)ₙ(C═O)—, —N*(R)(CR₂)ₙO(CR₂)ₘ—, —N*(R)(CR₂)ₙS(CR₂)ₘ—, —N*(R)(CR₂)ₙO—, —N*(R)(CR₂)ₙ(C═O)O—, —N*(R)(C═O)(CR₂)ₙO—, —N*(R)(C═O)(CR₂)ₙ(C═O)O—, —N*(R)(CR₂)ₙO(CR₂)ₘO—, —N*(R)(CR₂)ₙS(CR₂)ₘO—, —N*═CH(CR₂)ₙ—, —N*═CH(CR₂)ₙ(C═O)—, —N*═CH(CR₂)ₙO(CH₂)ₘ—, —N*═CH(CR₂)ₙS(CR₂)ₘ—, —N*═CH(CR₂)ₙO—, —N*═CH(CR₂)ₙ(C═O)O—, —N*═CH(CR₂)ₙO(CR₂)ₘO—, or —N*═CH(CR₂)ₙS(CR₂)ₘO—, wherein N* represents the nitrogen at the 2 position of the 2-amino monosaccharide;
R for each occurrence is independently hydrogen or lower alkyl;
m for each occurrence is independently a whole number selected between 1-20; and
n for each occurrence is independently a whole number selected between 1-20.

10. The redox polymer of claim 9, wherein the first repeating unit has the Formula IVa: 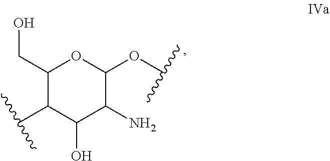
and the second repeating unit has the Formula IVb: 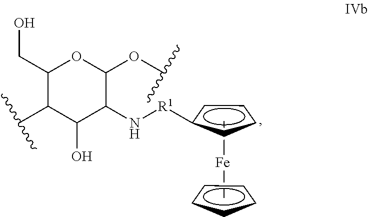
wherein
R¹ is —(C═O)—, —(C═O)(CH₂)ₙ—, —(C═O)(CH₂)ₙ(C═O)—, —(CH₂)ₙ(C═O)O—, or —(C═O)(CH₂)ₙO—.

11. The redox polymer of claim 10, wherein R¹ is —(C═O)—.

12. A sensor, comprising:
a substrate;
a first sensor electrode on the substrate;
a first sensing layer on the first sensor electrode, the first sensing layer comprising a first enzyme-containing polymer as defined in any one of claims 1 to 8; and
a reference electrode on the substrate.

13. The sensor according to claim 12, further comprising:
a second sensor electrode on the substrate; and
a second sensing layer on the second sensor electrode, the second sensing layer comprising a second enzyme-containing polymer as defined in any one of claims 1 to 8.

14. The sensor according to claim 12 or 13, wherein the first sensing layer or the second sensing layer has a thickness of in the range of about 0.010 mm to about 0.300 mm.

15. The sensor according to any one of claims 12 to 14, further comprising:
an elongated body portion having a body axis extending centrally through the elongated body portion; and
a tip portion having a tip axis extending centrally through the tip portion, the tip portion comprising the substrate,
wherein the tip portion is disposed adjacent the elongated body portion and at an obtuse angle of between 90° to 170° between the body axis and the tip axis.

16. The sensor according to claim 15, wherein the elongated body portion comprises a fluid reservoir and an actuator, and the tip portion further comprises an inflatable member, wherein a channel is disposed between the fluid reservoir and the inflatable member, and wherein the actuator is configured to deliver a volume of fluid in the fluid reservoir through the channel to the inflatable member to inflate the inflatable member.

17. The sensor according to claim 15, wherein the elongated body portion comprises a securing pin and the tip portion further comprises a moveable member, and wherein the securing pin is configured to secure the moveable member at an activated position

18. A monitor, comprising:
a receiver module configured to receive a sensor output of a sensor as defined in any one of claims 12 to 17 and a control output of another sensor as defined in any one of claims 12 to 17;
a processor module configured to receive a first metabolite concentration value corresponding to the sensor output and a first control value corresponding to the control output from the receiver module, wherein the processor module is configured to:
compare the first metabolite concentration value against the first control value; and
generate a first alarm signal on a condition that a difference between the first metabolite concentration value and the first control value is above a first pre-determined value.

19. The monitor according to claim 18, wherein the processor module is further configured to receive a second metabolite concentration value corresponding to the sensor output and a second control value corresponding to the control output from the receiver module, and wherein the processor module is further configured to:
compare the second metabolite concentration value against the second control value; and
generate a second alarm signal on a condition that a difference between the second metabolite concentration value and the second control value is above a second pre-determined value.

20. The monitor according to claim 19, wherein the processor module is further configured to trigger an alarm on a condition that the first alarm signal and/or the second alarm signal is generated, the alarm indicating a possibility of tissue failure.

21. The monitor according to claim 19 or 20, further comprising an output module in communication with the processor module, wherein the output module is configured to provide an indication of at least one of:
the first metabolite concentration value;
the first control value;
the second metabolite concentration value;
the second control value;
the difference between the first metabolite concentration value and the first control value;
the difference between the second metabolite concentration value and the second control value;
the difference between the first metabolite concentration value and the first control value with respect to the first pre-determined value; and
the difference between the second metabolite concentration value and the second control value with respect to the second pre-determined value.

22. A method of manufacturing a sensor, comprising:
providing a substrate;
forming a first sensor electrode on the substrate;
forming a first sensing layer on the first sensor electrode, the first sensing layer comprising a first enzyme-containing polymer as defined in any one of claims 1 to 8; and
forming a reference electrode on the substrate.

23. The method according to claim 22, further comprising:
forming a second sensor electrode on the substrate; and
forming a second sensing layer on the second sensor electrode, the second sensing layer comprising a second enzyme-containing polymer as defined in any one of claims 1 to 8.

24. A method for monitoring failure of a tissue on a patient, comprising the steps of:
(i) providing a first sensor according to claims 12 to 17 on or within said tissue, said first sensor being capable of detecting and measuring the amount of a first metabolite;
(ii) providing a second sensor according to claims 12 to 17 on a control region of said patient, said control region being separate from said tissue and wherein said second sensor is capable of detecting and measuring the amount of said first metabolite;
(iii) providing a third sensor according to claims 12 to 17 on or within said tissue, said third sensor being capable of detecting and measuring the amount of a second metabolite and wherein said third sensor is the same as or different from the first sensor;
(iv) providing a fourth sensor according to claims 12 to 17 on said control region of said patient, said fourth sensor being capable of detecting and measuring the amount of said second metabolite and wherein said fourth sensor is the same as or different from the second sensor;
(v) monitoring the amounts of said first metabolite measured by both said first and second sensors for a period of time; and
(vi) monitoring the amounts of said second metabolite measured by both said third and fourth sensors for a period of time,
wherein an at least 10% decrease in the amount of said first metabolite as measured by said first sensor as compared to the amount of said first metabolite as measured by said second sensor; and
at least 10% increase in the amount of said second metabolite as measured by said third sensor as compared to the amount of said second metabolite as measured by said fourth sensor, is indicative that said tissue is prone to failure.

25. A monitor, comprising:
a receiver module configured to receive a sensor output of a sensor according to claims 12 to 17 and a control output of another sensor according to claims 12 to 17;
a processor module configured to receive a first metabolite concentration value corresponding to the sensor output and a first control value corresponding to the control output from the receiver module, wherein the processor module is configured to:
compare the first metabolite concentration value against the first control value; and
generate a first alarm signal on a condition that a difference between the first metabolite concentration value and the first control value is above a first pre-determined value.

26. The monitor according to claim 25, wherein the processor module is further configured to receive a second metabolite concentration value corresponding to the sensor output and a second control value corresponding to the control output from the receiver module, and wherein the processor module is further configured to:
compare the second metabolite concentration value against the second control value; and
generate a second alarm signal on a condition that a difference between the second metabolite concentration value and the second control value is above a second pre-determined value.

27. The monitor according to claim 25, wherein the difference between the first metabolite concentration value and the first control value is at least 10% different from the first pre-determined value.

28. The monitor according to claim 26, wherein the difference between the second metabolite concentration value and the second control value is at least 10% different from the second pre-determined value.

29. A method for monitoring failure of a tissue on a patient, comprising the steps of:
(i) providing a first sensor according to claims 12 to 17 on or within said tissue, said first sensor being capable of detecting and measuring the amount of a first metabolite;
(ii) providing a second sensor according to claims 12 to 17 on a control region of said patient, said control region being separate from said tissue and wherein said second sensor is capable of detecting and measuring the amount of said first metabolite;
(iii) providing a third sensor according to claims 12 to 17 on or within said tissue, said third sensor being capable of detecting and measuring the amount of a second metabolite and wherein said third sensor is the same as or different from the first sensor;
(iv) providing a fourth sensor according to claims 12 to 17 on said control region of said patient, said fourth sensor being capable of detecting and measuring the amount of said second metabolite and wherein said fourth sensor is the same as or different from the second sensor;
(v) monitoring the amounts of said first metabolite measured by both said first and second sensors for a period of time; and
(vi) monitoring the amounts of said second metabolite measured by both said third and fourth sensors for a period of time,
wherein an at least 10% decrease in the amount of said first metabolite as measured by said first sensor as compared to the amount of said first metabolite as measured by said second sensor; and
at least 10% increase in the amount of said second metabolite as measured by said third sensor as compared to the amount of said second metabolite as measured by said fourth sensor, is indicative that said tissue is prone to failure.

30. A method for monitoring failure of a tissue on a patient, comprising the steps of:
(i) providing a first sensor according to claims 12 to 17 on or within said tissue, said first sensor being capable of detecting and measuring the amount of a first metabolite;
(ii) providing a second sensor according to claims 12 to 17 on a control region of said patient, said control region being separate from said tissue and wherein said second sensor is capable of detecting and measuring the amount of said first metabolite;
(iii) providing a third sensor on according to claims 12 to 17 or within said tissue, said third sensor being capable of detecting and measuring the amount of a second metabolite and wherein said third sensor is the same as or different from the first sensor;
(iv) providing a fourth sensor according to claims 12 to 17 on said control region of said patient, said fourth sensor being capable of detecting and measuring the amount of said second metabolite and wherein said fourth sensor is the same as or different from the second sensor;
(v) monitoring the amounts of said first metabolite measured by both said first and second sensors for a period of time; and
(vi) monitoring the amounts of said second metabolite measured by both said third and fourth sensors for a period of time,
wherein the amount of said first metabolite as measured by said first sensor and the amount of said first metabolite as measured by said second sensor are substantially the same; and
at least 10% increase in the amount of said second metabolite as measured by said third sensor as compared to the amount of said second metabolite as measured by said fourth sensor, is indicative that said tissue is prone to failure.

31. A method for monitoring failure of a tissue on a patient, comprising the steps of:
(i) providing a first sensor according to claims 12 to 17 on or within said tissue, said first sensor being capable of detecting and measuring the amount of a first metabolite;
(ii) providing a second sensor according to claims 12 to 17 on a control region of said patient, said control region being separate from said tissue and wherein said second sensor is capable of detecting and measuring the amount of said first metabolite;
(iii) providing a third sensor according to claims 12 to 17 on or within said tissue, said third sensor being capable of detecting and measuring the amount of a second metabolite and wherein said third sensor is the same as or different from the first sensor;
(iv) providing a fourth sensor according to claims 12 to 17 on said control region of said patient, said fourth sensor being capable of detecting and measuring the amount of said second metabolite and wherein said fourth sensor is the same as or different from the second sensor;
(v) monitoring the amounts of said first metabolite measured by both said first and second sensors for a period of time; and
(vi) monitoring the amounts of said second metabolite measured by both said third and fourth sensors for a period of time,
wherein an at least 10% decrease in the amount of said first metabolite as measured by said first sensor as compared to the amount of said first metabolite as measured by said second sensor; and
the amount of said second metabolite as measured by said third sensor and the amount of said second metabolite as measured by said fourth sensor are substantially the same, is indicative that said tissue is prone to failure.

32. A method for monitoring failure of a tissue on a patient, comprising the steps of:
(i) providing a first sensor according to claims 12 to 17 on or within said tissue, said first sensor being capable of detecting and measuring the amount of a first metabolite;
(ii) providing a second sensor according to claims 12 to 17 on a control region of said patient, said control region being separate from said tissue and wherein said second sensor is capable of detecting and measuring the amount of said first metabolite;
(iii) providing a third sensor on according to claims 12 to 17 or within said tissue, said third sensor being capable of detecting and measuring the amount of a second metabolite and wherein said third sensor is the same as or different from the first sensor;
(iv) providing a fourth sensor according to claims 12 to 17 on said control region of said patient, said fourth sensor being capable of detecting and measuring the amount of said second metabolite and wherein said fourth sensor is the same as or different from the second sensor;
(v) monitoring the amounts of said first metabolite measured by both said first and second sensors for a period of time; and
(vi) monitoring the amounts of said second metabolite measured by both said third and fourth sensors for a period of time,
wherein an at least 10% decrease in the amount of said first metabolite as measured by said first sensor as compared to the amount of said first metabolite as measured by said second sensor; and
an at least 10% decrease in the amount of said second metabolite as measured by said third sensor as compared to the amount of said second metabolite as measured by said fourth sensor, is indicative that said tissue is prone to failure.

33. A method for monitoring failure of a tissue on a patient, comprising the steps of:
(i) providing a first sensor according to claims 12 to 17 on or within said tissue, said first sensor being capable of detecting and measuring the amount of a first metabolite;
(ii) providing a second sensor according to claims 12 to 17 on a control region of said patient, said control region being separate from said tissue and wherein said second sensor is capable of detecting and measuring the amount of said first metabolite;
(iii) providing a third sensor according to claims 12 to 17 on or within said tissue, said third sensor being capable of detecting and measuring the amount of a second metabolite and wherein said third sensor is the same as or different from the first sensor;
(iv) providing a fourth sensor according to claims 12 to 17 on said control region of said patient, said fourth sensor being capable of detecting and measuring the amount of said second metabolite and wherein said fourth sensor is the same as or different from the second sensor;
(v) monitoring the amounts of said first metabolite measured by both said first and second sensors for a period of time; and
(vi) monitoring the amounts of said second metabolite measured by both said third and fourth sensors for a period of time,
wherein an at least 10% increase in the amount of said first metabolite as measured by said first sensor as compared to the amount of said first metabolite as measured by said second sensor; and
an at least 10% increase in the amount of said second metabolite as measured by said third sensor as compared to the amount of said second metabolite as measured by said fourth sensor, is indicative that said tissue is prone to failure.
